(19) United States
(12) Patent Application Publication
(10) Pub. No.: US 2008/0021502 A1
(43) Pub. Date: Jan. 24, 2008

(54) Systems and methods for automatic symmetry identification and for quantification of asymmetry for analytic, diagnostic and therapeutic purposes

(75) Inventors: Celina Imielinska, Princeton, NJ (US); Anthony D'Ambrosio, Ridgewood, NJ (US); Xin Liu, New York, NY (US); Michael Sughrue, San Francisco, CA (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(73) Assignee: THE TRUSTEES OF COLUMBIA UNIVERSITY IN THE CITY OF NEW YORK

(21) Appl. No.: 11/706,128

(22) Filed: Feb. 12, 2007

Related U.S. Application Data
(63) Continuation-in-part of application No. 10/872,666, filed on Jun. 21, 2004.
(60) Provisional application No. 60/772,091, filed on Feb. 10, 2006.
(60) Provisional application No. 60/838,521, filed on Aug. 16, 2006.

(51) Int. Cl. A61N 1/39

(52) U.S. Cl. 607/1

(57) ABSTRACT

Methods and related computational techniques are presented for analyzing images from various scan modalities, such as computerized tomography (“CT”). The methods convert image values to relative differences, to highlight side-to-side asymmetry. The conversion may be performed by comparing a small region of the scan to the corresponding region in the contralateral hemisphere, quantifying the degree of relative difference using statistical techniques, and representing this quantity of relative difference in a two dimensional or three dimensional relative difference map. In exemplary embodiments the method involves assigning an axis or plane of symmetry to a medical image, computing, using the image data, at least one relative difference map based on a comparison of two substantially symmetrical areas around the axis of symmetry, and generating a representation of any relative difference between the two symmetrical areas. In exemplary embodiments of the present invention the 3D orientation of a volumetric representation of an organ or anatomical area can be realigned within a scanner co-ordinate system. An inertia matrix can be computed on the sampled organ or structure, and principal axes can be can be derived from the eigenvectors of the inertia matrix. In alternate exemplary embodiments of the present invention, a volume, such as, for example, of a brain, can be represented as a re-parameterized surface point cloud. The interior contents can be removed, thus decomposing a symmetry plane computation problem into a surface matching routine. A search for a best matching surface can be implemented in a multi-resolution paradigm so as to optimize computational time. Subsequent to processing using either technique, in exemplary embodiments of the present invention a spatial affine transform can be applied to rotate the 3D images and align them within the co-ordinate system of the scanner. The corrected organ volume, for example, a brain, can then be re-sliced such that each planar image represents the organ at the same axial level.

### CROSS REFERENCE TO OTHER APPLICATIONS

This application is a continuation-in-part of U.S. patent application Ser. No. 10/872,666, filed on Jun. 21, 2004, the disclosure of which is hereby incorporated herein by reference. Additionally, this application claims the benefit of U.S. Provisional Patent Applications Nos. 60/772,091, filed on Feb. 10, 2006, and 60/838,521, filed on Aug. 16, 2006, the disclosure of each of which is hereby incorporated herein by this reference.

### TECHNICAL FIELD

The present invention is directed to improved systems and methods for performing medical and other imaging, and more particularly to systems and methods for automatic symmetry identification and for quanitification of assymetry, including preprocessing by performing automatic orientation and tilt correction, for various analytic, diagnostic and therapeutic purposes.

### BACKGROUND OF THE INVENTION

Computed tomography (CT), positron emitted tomography (PET), magnetic resonance imaging (MRI) and other radiological imaging techniques are well known in medical diagnostics and other imaging based applications. Recent advances in the image processing techniques associated with these technologies has provided medical practitioners with the ability to obtain structural, physiological and functional image data from these tests. The image processing software used in conjunction with MRI and CT allows a user to acquire images of a particular region and process image data to generate physiological image data relating to perfusion parameters.

This perfusion data may be utilized to assess the viability of an area of interest such as certain regions of human tissue by determining various perfusion parameters such as a mean transit time (MTT), a cerebral blood flow (CBF), and a cerebral blood volume (CBV). The image processing software calculates changes in these parameters to generate physiological images of specified regions of human anatomy. Medical practitioners may use these perfusion weighted images to aid in patient diagnosis by comparing the currently acquired images with any known physiological norms or previous test results to determine any differences.

Currently, medical imaging technologies operate by first generating a grayscale image of the digitally converted signals to construct a pixel-based image of an object of interest. Subsequently, color may be introduced to help highlight areas of varying intensity to facilitate image evaluation. However, image evaluation is a complex process that may be adversely affected by a number factors, such as imperfect images, low resolution images, the limitations of human perception or perceptual bias. Such factors may introduce the possibility that clinical error may occur, which can result in an incorrect patient diagnosis.

In one particular example, Perfusion-Weighted Computed Tomography (CTP) is a relatively recent innovation that utilizes a set of successive axial head CT images to track the time course of signal from an administered bolus of intravenous contrast. These images may be processed using either deconvolution or maximum slope algorithms to extrapolate a numerical value for cerebral blood flow (CBF). While “bolus tracking” methods may provide accurate quantification of CBF under controlled conditions, variability in cardiac function, systemic blood pressure, and cerebrovascular tone often seen in the setting of acute SAH makes quantitative and qualitative assessment of these studies both difficult and potentially hazardous.

While CTP has found some utility in the diagnosis and management of ischemic stroke, its potential use in the diagnosis and management of delayed cerebral vasospasm (CVS) has not been investigated. Furthermore, because this imaging technique is both fast and noninvasive, it is an ideal diagnostic test for this unstable patient population. Unfortunately, due to the inherent variability described above, there is no currently accepted, standardized method of interpreting these scans. Most commonly, scans are interpreted using the qualitative detection of gross side-to-side asymmetry of CBF, an approach that lends itself to misdiagnosis and potential failure to treat CVS. Recent work with CTP has focused on the development of methods to quantitatively analyze CTP images. Most of these approaches utilize the region of interest (ROI) method. In this approach, the clinician circles an ROI on the post-processed CTP image, and the mean CBF is compared to that of the corresponding ROI in the contralateral hemisphere to detect asymmetry. A growing body of data supports improved safety and efficacy of this approach in the setting of acute ischemic stroke.

Accordingly, in view of the foregoing, it would be desirable to provide methods and apparatus for performing electronic image processing that do not rely solely on human experts to evaluate medical images.

It would therefore also be desirable to provide methods and apparatus for electronic imaging that facilitates the assessment of images, and the relative differences between portions of images, and in particular structural, physiological and functional image data, and more particularly for perfusion weighted imaging data, to aid in patient diagnosis.

Moreover, however, symmetry based analysis cannot be precisely carried out when the 3D orientation of an organ under analysis, such as, for example, the brain, is misaligned, a common occurrence in clinical practice. In fact, many images of the brain are often clinically unreadable due to misalignment of a patient's head in an imaging scanner.

Thus, it would additionally be desirable to have systems and techniques to automatically compute the symmetry plane and correct the 3D orientation of images, such as, for example, patient brain images.

### SUMMARY OF THE INVENTION

Methods and related computational techniques suitable for use in imaging software are provided for evaluating a medical image represented by image data. In exemplary embodiments of the present invention the method involves assigning an axis or plane of symmetry to the medical image, computing, using the image data, at least one relative difference map based on a comparison of two substantially symmetrical areas around the axis of symmetry, and generating a representation of any relative difference between the two symmetrical areas. In some embodiments, the image data can be acquired by scanning a region of interest, such as, for example, by performing a computed tomography or other radiological scan of a body.

The axis or plane of symmetry may be assigned by a user through a user interface to the software program, or automatically by the program based on the image data or physical criteria. The relative difference map may be represented as a two or three dimensional color image illustrating the relative difference between the two substantially symmetrical areas, as a histogram representing the relative difference between the two substantially symmetrical areas, or by any other convenient way to review of the results of the computation.

In some embodiments, the relative difference map is determined by computing a similarity discrepancy between the two substantially symmetrical areas about an axis or plane of symmetry. One known technique useful in performing such a statistical calculation is the Kolmogorov-Smirnov test. This computation may be performed by first defining at least two windows in the image data, each window representing at least a portion of one of the symmetrical areas for which at least one relative difference map is to be computed. The windows may be defined by positioning each window in substantially equidistant locations from, and positioned symmetrically with respect to, the assigned axis or plane of symmetry. In some embodiments, a composite axis or plane of symmetry (e.g., an average or other composite representation of possible axes or planes) can be used in situations where a single axis or plane is insufficient or does not provide a comprehensive image or the comparison information desired. Such windows can be user defined or preset, for example, and can contain n×n pixels of image data, depending on a number of factors such as, for example, noise suppression or resolution. In some embodiments, good results can be obtained where n=9.

In accordance with some embodiments of the present invention, methods and related computational techniques are provided for analyzing images such as post-processed CTP images obtained from a standard perfusion CT software package, such as, for example, a Siemens Medical Solutions package. This method converts CBF values to relative differences, which represent meaningful side-to-side asymmetry. In one embodiment, this conversion can be performed by comparing a small region of the scan to the corresponding region in the contralateral hemisphere, quantifying the degree of relative difference, and representing this quantity of relative difference in a two dimensional or three dimensional Relative Difference Map (“RDM”).

In exemplary embodiments of the present invention, the method can involve analyzing the amount of relative difference in both brain hemispheres and six major vascular territories to assess the degree of hypoperfusion in such regions. In this embodiment, a simplified model of the human brain can be defined as a symmetric object if corresponding regions of both hemispheres have comparable structural similarity and CBF equivalence. Such a model is generally supported by the following assumptions, made based on widely accepted human brain anatomy and physiology characteristics: (1) in normal cases, the axial CT images of the left and right hemispheres are structurally symmetric and comparable, and there should be no significant relative blood flow difference between the two hemispheres; (2) in abnormal cases, the left and right hemispheres are still structurally symmetric and comparable, but there is significant relative blood flow difference between the two hemispheres that can be detected using CTP images. The method can, for example, be automated, and can, for example, provide a better and more stable analysis of the perfusion parameters of unstable patients such as those, for example, with subarachnoid hemorrhage (SAH).

In exemplary embodiments of the present invention, the 3D orientation of a volumetric representation of an organ or anatomical area can be realigned within a scanner co-ordinate system. An inertia matrix can be computed on the sampled organ or structure, and principal axes can be derived from the eigenvectors of the inertia matrix. In alternate exemplary embodiments of the present invention, a volume, such as, for example, of a brain, can be represented as a re-parameterized surface point cloud. The interior contents can be removed, thus decomposing a symmetry plane computation problem into a surface matching routine. A search for a best matching surface can be implemented in a multi-resolution paradigm so as to optimize computational time. Subsequent to processing using either technique, in exemplary embodiments of the present invention a spatial affine transform can be applied to rotate the 3D images and align them within the co-ordinate system of the scanner. The corrected organ volume, for example, a brain, can be re-sliced such that each planar image represents the organ at the same axial level.

### BRIEF DESCRIPTION OF THE DRAWINGS

FIGS. 1(a)-(f) illustrate an exemplary relative difference map (“RDM”) method according to an exemplary embodiment of the present invention;

FIGS. 1A(a)-(f) depict FIGS. 1(a)-(f) as larger images;

FIG. 1A is an exemplary process flow diagram corresponding to the exemplary method illustrated in FIG. 1;

FIG. 2 depicts a set of hypothesized histograms quantified from the relative difference maps of each of normal patients, patients with increased vasospasm risk, angiographic vasospasm, and ischemic stroke, respectively;

FIGS. 3(a)-(f) depict various image outputs obtained from applying the exemplary RDM method of FIG. 1 to a “normal” patient according to an exemplary embodiment of the present invention;

FIGS. 3A(a)-(f) depict FIGS. 3(a)-(f) as larger images;

FIGS. 4(a)-(f) depict various image outputs obtained from applying the exemplary RDM method of FIG. 1 to an “injured” patient according to an exemplary embodiment of the present invention;

FIGS. 4A(a)-(f) depict FIGS. 4(a)-(f) as larger images;

FIG. 5(a) depicts an exemplary CBF image constructed with a Siemens Medical Solutions perfusion software package according to an exemplary embodiment of the present invention;

FIG. 5(b) depicts the original gray-scale image use to construct the Relative Difference Map, shown in FIG. 5(c) within the Siemens' interface;

FIG. 5(c) is a color relative difference map image generated from the image in FIG. 5(b), according to an exemplary embodiment of the present invention;

FIGS. 5A(a)-(c) depict FIGS. 5(a)-(c) as larger images;

FIGS. 6(a)-(c) illustrate the computation of a convex hull from a set of points in 2D according to an exemplary embodiment of the present invention;

FIGS. 6A(a)-(c) depict FIGS. 6(a)-(c) as larger images;

FIG. 7 illustrates ray casting and angle step size relative to maximum ray length according to an exemplary embodiment of the present invention;

FIG. 8 illustrates increasing a ray's thickness according to an exemplary embodiment of the present invention;

FIGS. 9(a)-(b) illustrate a convex hull and a blob for an exemplary image according to an exemplary embodiment of the present invention;

FIGS. 9A(a)-(b) depict FIGS. 9(a)-(b) as larger images;

FIGS. 10(a)-(b) depict an exemplary input image and four quadrants derived form the centroids computed from an associated blob according to an exemplary embodiment of the present invention;

FIGS. 10A(a)-(b) depict FIGS. 10(a)-(b) as larger images;

FIGS. 11(a)-(b) illustrate unwrapped data and a corresponding input image divided into six vascular territories according to an exemplary embodiment of the present invention;

FIGS. 11A(a)-(b) depict FIGS. 11(a)-(b) as larger images;

FIGS. 12(a)-(b) illustrate an exemplary image processed by a 9×9, window and an associated RDM for the image according to an exemplary embodiment of the present invention;

FIGS. 12A(a)-(b) depict FIGS. 12(a)-(b) as larger images;

FIG. 13(a) depicts an exemplary color relative difference map and associated histograms of the left and right hemispheres of a patient with normal CBF generated according to an exemplary embodiment of the present invention;

FIG. 13(b) depicts the relative difference map illustrated in FIG. 13(a) with six assigned cranial vascular territories and six associated histograms according to an exemplary embodiment of the present invention;

FIGS. 13A(a)-(b) depict FIGS. 13(a)-(b) as larger images;

FIG. 14(a) depicts an exemplary color relative difference map and associated histograms of the left and right hemispheres of a patient with ischemic stroke generated according to an emplary embodiment of the present invention;

FIG. 14(b) shows the relative difference map illustrated in FIG. 14(a) with six assigned cranial vascular territories and six associated histograms according to an exemplary embodiment of the present invention;

FIGS. 14A(a)-(b) depict FIGS. 14(a)-(b) as larger images;

FIG. 15(a) depicts an exemplary color relative difference map and associated histograms of the left and right hemispheres of a patient with ASH generated according to an examplary embodiment of the present invention;

FIG. 15(b) shows the relative difference map illustrated in FIG. 15(a) with six assigned cranial vascular territories and six associated histograms according to an exemplary embodiment of the present invention;

FIGS. 15A(a)-(b) depict FIGS. 15(a)-(b) as larger images;

FIGS. 16(a)-(c) depict the results of an automated RDM/Histogram generation algorithm according to an exemplary embodiment of the present invention (the patient is the same as that depicted in FIGS. 14);

FIGS. 16A(a)-(c) depict FIGS. 16(a)-(c) as larger images;

FIG. 17 is a process flow diagram for an exemplary segmentation scheme according to an exemplary embodiment of the present invention;

FIG. 18(a) depicts an axis of symmetry in r-O space, and FIG. 18(b) depicts symmetry measure along a 27π spatial orientation according to an exemplary embodiment of the present invention;

FIGS. 19(a)-(c) depict exemplary perfusion-weighted computed tomography images for a subject with ischemic stroke, according to an exemplary embodiment of the present invention;

FIGS. 20(a)-(d) depict an exemplary rat stroke segmentation according to an exemplary embodiment of the present invention;

FIGS. 20A(a)-(d) depict FIGS. 20(a)-(d) as larger images;

FIGS. 21(a)-(c) illustrate an exemplary tumor segmentation from MR patient data according to an exemplary embodiment of the present invention;

FIGS. 21A(a)-(c) depict FIGS. 21(a)-(c) as larger images;

FIGS. 22(a)-(c) depict an exemplary segmentation of stroke in patient MR Diffusion Weighted Images (DUI) according to an exemplary embodiment of the present invention;

FIGS. 22A(a)-(c) depict FIGS. 22(a)-(c) as larger images;

FIG. 23 depicts an exemplary segmentation of a tumor in MR patient data according to an exemplary embodiment of the present invention;

FIG. 24 is a table comparing segmentation results between an exemplary RDM method according to the present invention and those obtained from a fuzzy connectedness algorithm;

FIG. 25 is a table comparing accuracy measurements for the two methods shown in FIG. 24;

FIGS. 26 depict an exemplary segmentation and validation of stroke regions in MR rat brain data according to an exemplary embodiment of the present invention;

FIG. 27 depicts Pitch, Roll and Yaw angles of the brain;

FIG. 28(a) illustrates one principal axis (shown as a yellow vector pointing into the side of the head above the ear) in volume rendered MRI images, and FIG. 28(b) demonstrates the relationship between the symmetry plane and the principal axis, according to an exemplary embodiment of the present invention;

FIG. 29 illustrates automatic correction of a tilted volume according to an exemplary embodiment of the present invention;

FIG. 30 illustrates automatic computation of three planes that are orthogonal to three distinctive principal axes according to an exemplary embodiment of the present invention;

FIGS. 31(a)-(f) further illustrate automatic correction of CT scans with tilted volumes according to an exemplary embodiment of the present invention;

FIGS. 31A depict the columns of FIG. 31, i.e., FIGS. 31(a) and (d),(b) and (e), and (c) and (f), respectively, as larger images;

FIGS. 32(a) and (b) illustrate correction of misaligned CT scans and MRI scans according to an exemplary embodiment of the present invention;

FIGS. 32A(a) and (b) depict FIGS. 322(a) and (b) as larger images;

FIG. 33 illustrates correction of MR images with big brain lesion and certain level of brain shift according to an exemplary embodiment of the present invention;

FIG. 34 illustrates the use of a surface normal to characterize the symmetry plane in volumetric neuron-images according to an exemplary embodiment of the present invention;

FIG. 35 illustrates a 3D brain volume represented into a surface point cloud, where each location on the surface is parameterized by its elevation (latitude), azimuth (longitude) and radius {(α,θ,r)}, according to an exemplary embodiment of the present invention;

FIG. 36 illustrates the operation of an exemplary surface matching algorithm according to an exemplary embodiment of the present invention; and

FIG. 37 depicts exemplary original and corrected MRI scans according to an exemplary embodiment of the present invention;

The patent or application contains at least one drawing executed in color. Copies of this patent or patent application publication with color drawing(s) will be provided by the Office upon request and payment of the necessary fee.

Nonetheless, because some readers will not have the color drawings available, the description will also endeavor to describe the drawings and the images they depict in a color-neutral manner, which may create apparent redundancies of description.

### DETAILED DESCRIPTION OF THE INVENTION

The following description is divided into six parts. The first part is a general description of methods according to exemplary embodiments of the present invention. The remaining five parts describe actual exemplary studies performed by the inventors hereof implementing various exemplary embodiments of the present invention.

I. Overview and General Description

Although the present invention is sometimes described below in connection with images obtained from CT scanning, it should be understood that the principles and novel concepts described herein can be implemented using any of a variety of imaging modalities, such as, for example, MRI, MRA, or PET.

In such images, colors representing intensities (or intensity differences) can, for example, be mapped using threshold values that correspond to a physiological threshold. For example, to facilitate the detection of tissues in which an ischemia is occurring, a predetermined color, such as, for example, blue, can be mapped to threshold levels selected to show where the ischemia is occurring. Other colors, such as green, for example, can be mapped to threshold areas of lower intensity in a reconstructed image representing healthy tissues, and yet another color such as red, for example, can be mapped to areas having intensities characteristic of blood vessels. Such mappings can be useful for the assessment of blood flow.

Once parametric and image information have been captured as described above, subsequent data processing may be performed in a computer (using, for example, symmetry analysis software, constructed as described herein) for subjects which have symmetrical or substantially symmetrical counterparts such as, for example, human anatomy or other naturally occurring symmetrical objects. Such processing may produce, among other things, an asymmetry determination and relative difference maps (described in more detail below) that can illustrate relative differences between two objects with a high degree of precision and which may be used to supplement or confirm any qualitative symmetry analysis performed by human analysis.

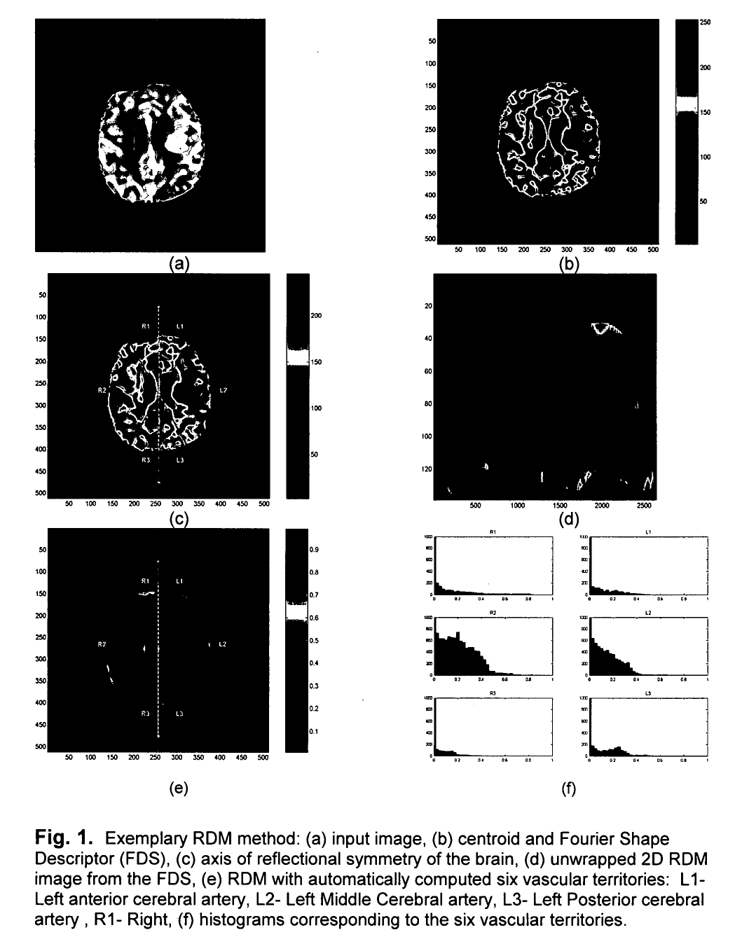

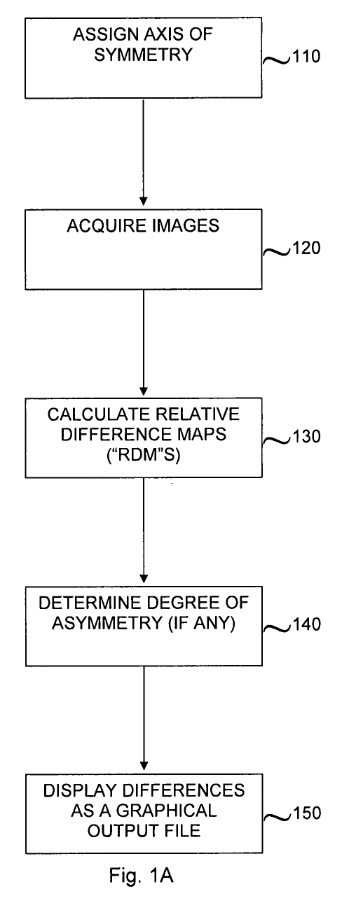

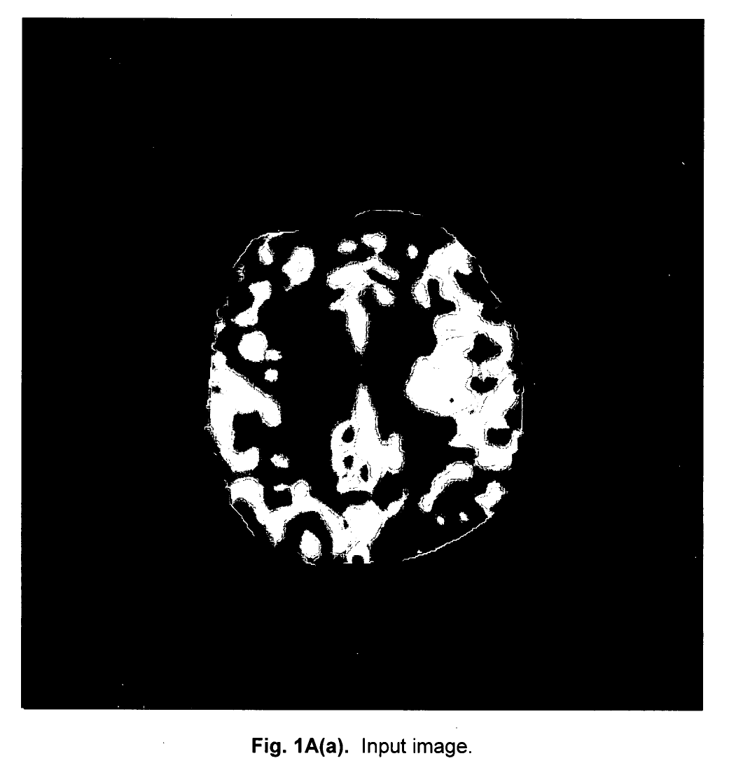

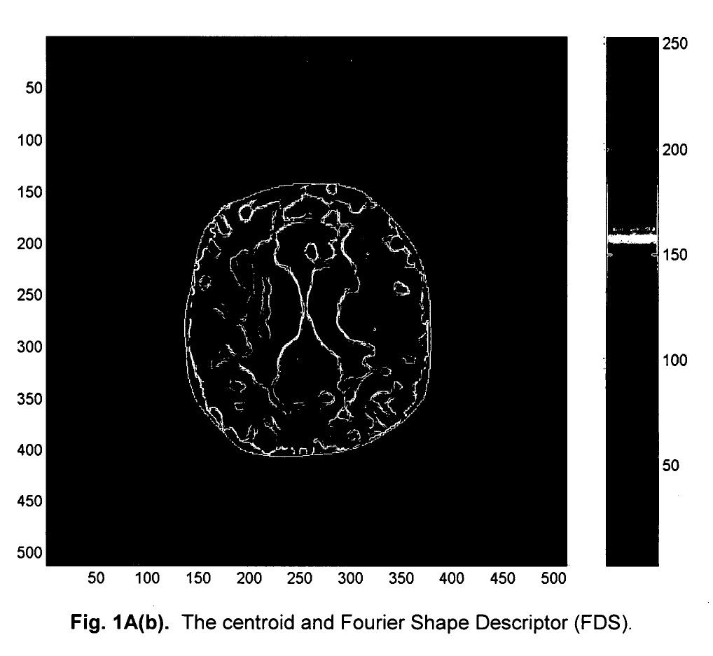

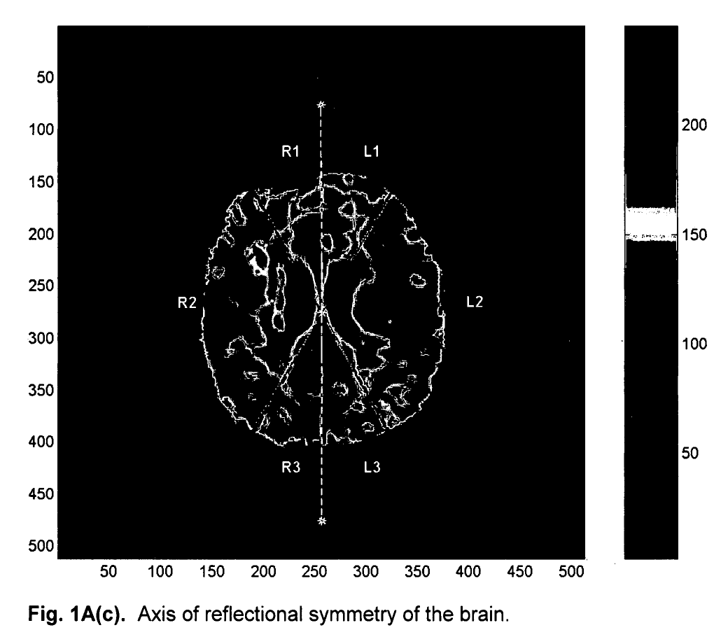

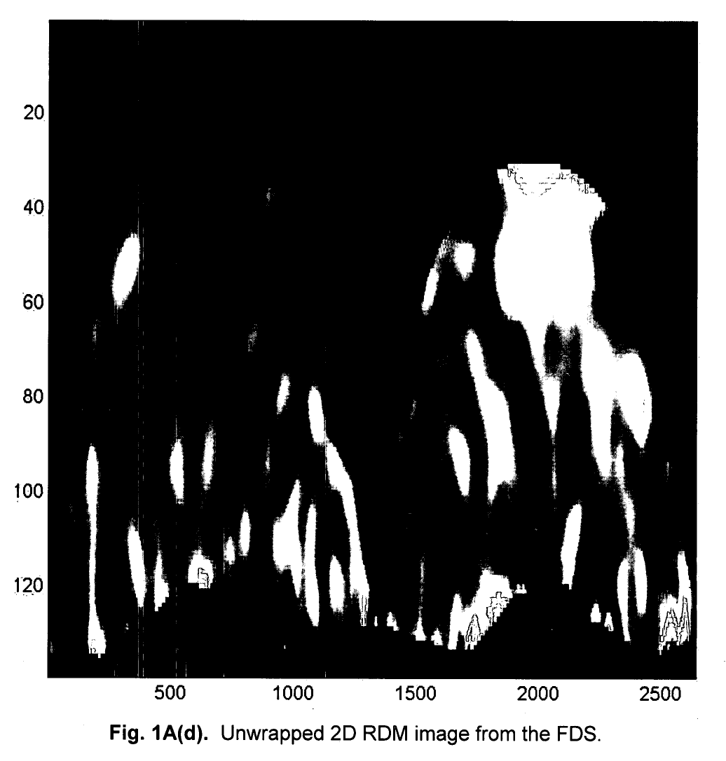

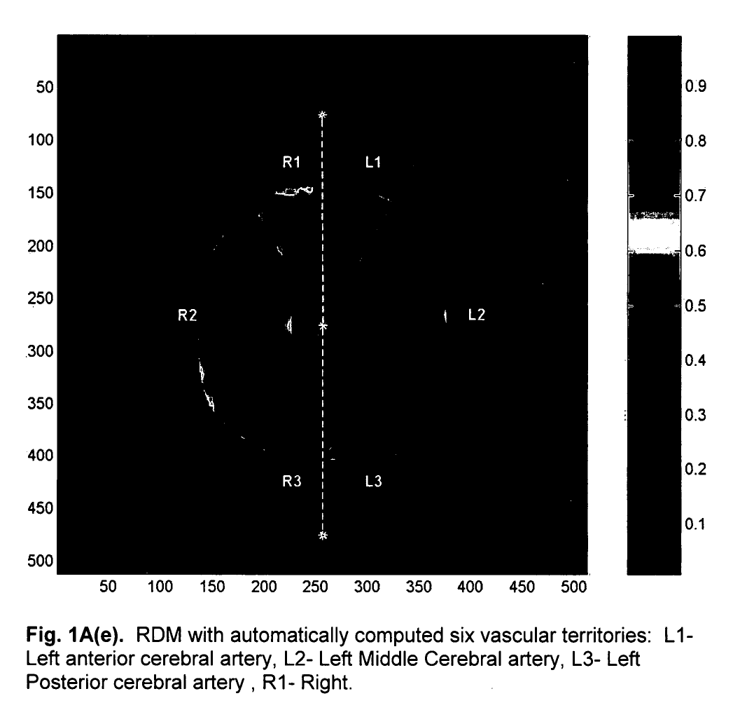

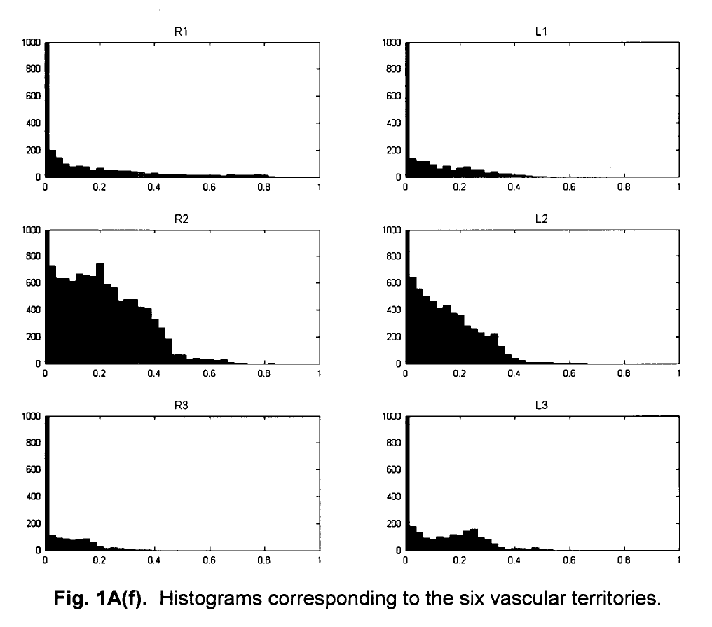

In general, a method according to the present invention can include processing as depicted in the flow chart of FIG. 1A. As shown, at 110 a plane of symmetry can be assigned for a subject to be scanned. In some embodiments, this step can be performed prior to the image capture process begins so that the subject can be properly aligned within the scanning system, such as a CT scanning device. This helps eliminate the capture of undesired asymmetric data and thereby reduces clinical error. In other embodiments, however, an entire region may be scanned first, and the plane of symmetry may be assigned later based on the acquired data or on an area of interest. Often, the plane of symmetry is chosen such that the subject image is separated into two distinct symmetrical regions. The plane of symmetry may be assigned, for example, by comparison software resident in a computer based on certain physical or other known orientation criteria.

At this point, the scanning system acquires the necessary perfusion-weighted images (at 120) and prepares the acquired data for postprocessing. This may be performed by software in a Siemens Medical Solutions package, for example. Next, at 130, an axis of symmetry can be assigned and a mathematical analysis can be used to compare the selected regions with respect to the assigned axis of symmetry in accordance with the principles of the present invention. Such mathematical analysis can include, for example: 1) computing the convex hull of the input image and its corresponding Fourier shape descriptor, 2) computing a series of centroids in the convex hull that may define the axis of symmetry; 3) “unwrapping” the convex hull over a rectangular map with the axis of symmetry in the middle (converting the convex image to a substantially rectangular or square one, similar to creating a Mercator projection of the convex hull); 4) optionally normalizing the unwrapped image; 5) analyzing pixel by pixel symmetry using binary and/or gradient image data; and 6) analyzing and quantifying the degree of symmetry (such as, for example, with RDMs, histograms, etc.). This exemplary analysis is discussed in more detail below.

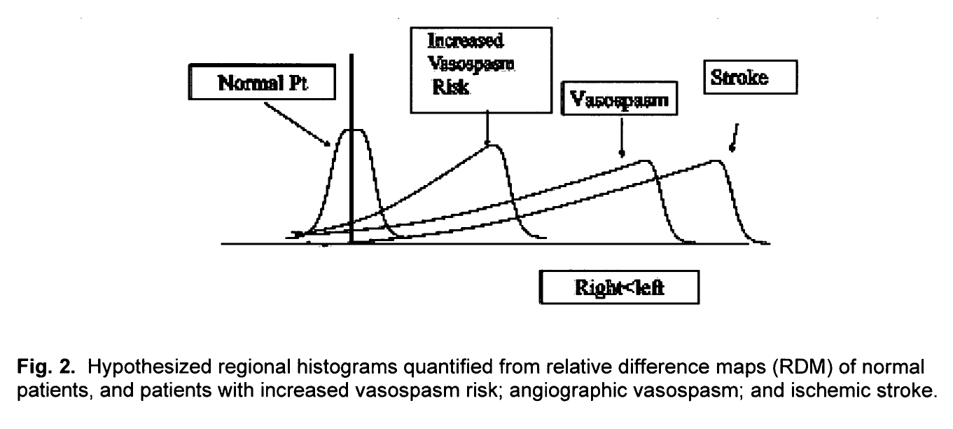

The regions to analyze can, for example, be selected by a user based on a particular interest, and the analysis can be performed iteratively for varying parts of the region, or for the image in its entirety taken region by region. The mathematical analysis may include any method that determines the difference between two populations of data points, although a method that does not require a normal distribution of data points is preferred. Each value that appears on the RDM indicates a point of asymmetry. Any detected asymmetries can, for example, be plotted in two and/or three dimensional maps with results also produced in the form of relative difference maps or other representations having a similar functionality. At 140, for example, the degree of asymmetry in specific regions of interest may be quantified and analyzed mathematically by, for example, generating histograms that plot the number and frequency of differential points between the selected regions (this is shown in FIG. 2, and discussed in greater detail below). At 150, the quantitative analysis performed at 140 can be linked to a specific output format suitable for the desired application. Such an application can include, for example, a graphical display program that illustrates the asymmetries present in portions of a human body.

In one exemplary embodiment, the above described processing can be carried out automatically by software implemented in a digital computer. Such an automated image acquisition and comparison method can be used to aid or supplement the assessment of symmetry (or asymmetry) of an object by a human specialist, whose analytical ability is generally limited by the boundaries of human acuity. Thus, in exemplary embodiments of the present invention, the method can provide a medical practitioner with the detailed information necessary to make correct and accurate diagnostic decisions even when the symptoms are beyond what would normally be noticed by a human observer. It can also provide the medical community with a computer based diagnostic tool to improve diagnostic decisions, as well as and a teaching and training tool to help medical students recognize symmetries or asymmetries in patients.

In exemplary embodiments of the present invention, computation of the convex hull, as described above, can be determined through the use of bounding functions. For example, to obtain a function that bounds a region in an image described by points on the outer most edge of a region, rays can be generated from a centroid (cₓ,c_{y},) of the region outward at specific increasing angles θ. To obtain a valid sampling, theta can be increased at each step, as indicated below in Equation 5:

$$Δ θ < sin {-}^{1} ( \frac{1}{2 {R}_{max}} ) \qquad ( 5 )$$

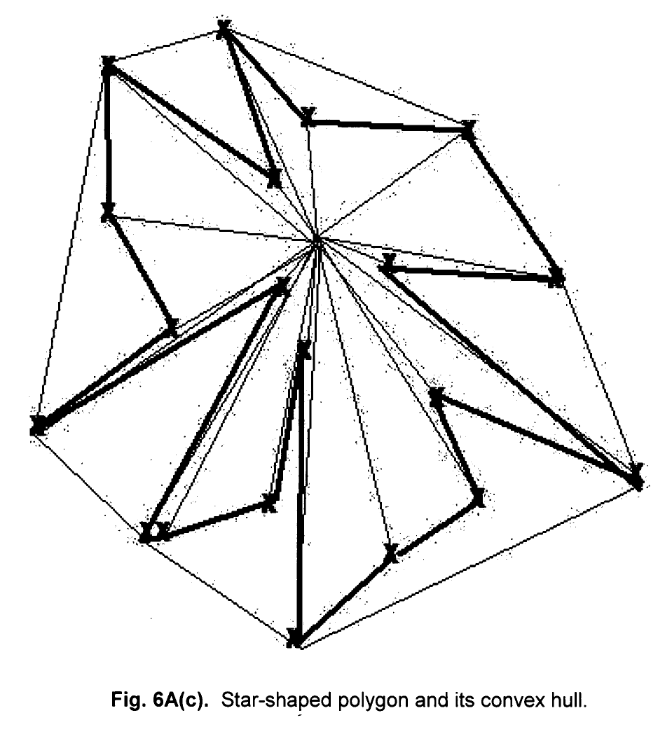

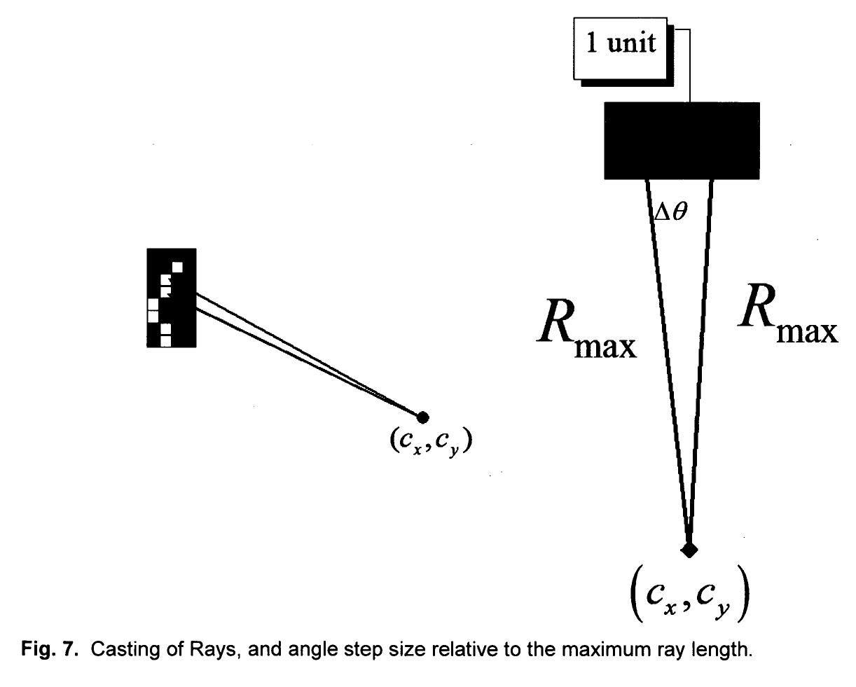

This is illustrated in FIG. 7.

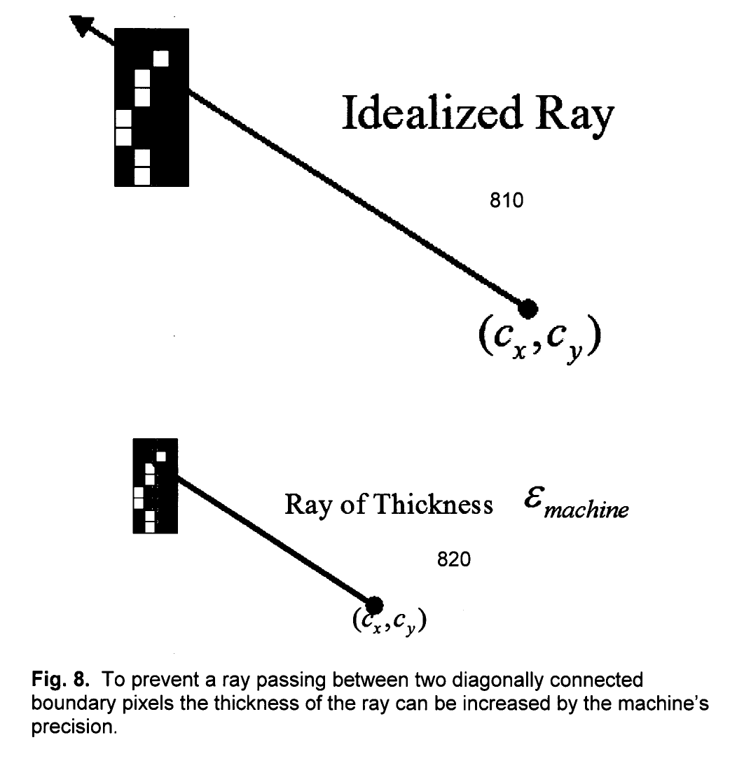

Some rays may pass through a singularity in a boundary, meaning, that it is possible, although unlikely, that a ray will pass between two boundary pixels that are diagonally connected. To prevent this from happening a ray's thickness can be increased, for example, on the order of the precision of the scanning machine, as shown in FIG. 8.

By recording the length of the ray at each angle, a description of a bounding function R(θ) of the region as a function of θ can be generated. Moreover, this function may not have desirable properties (e.g., it not may not be convex).

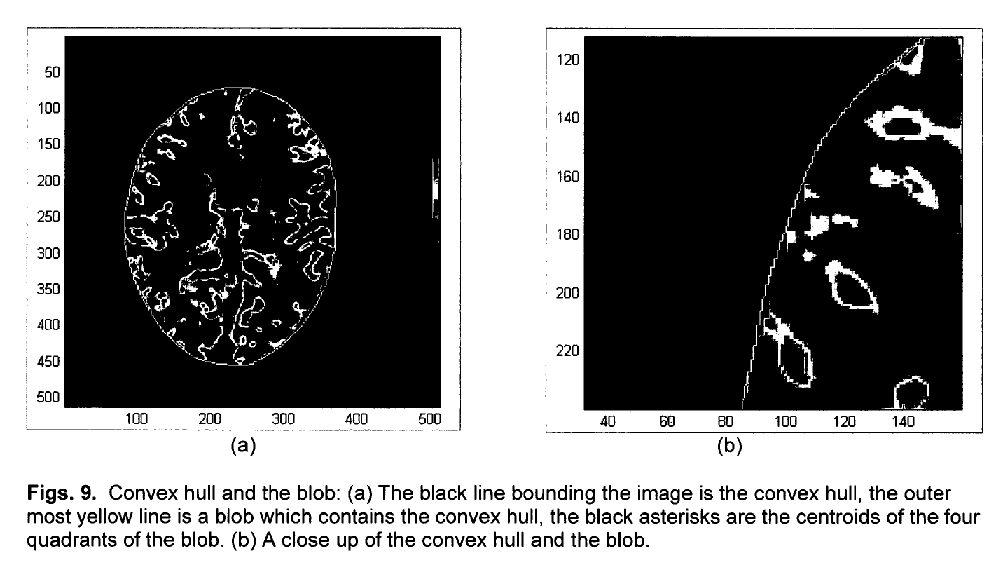

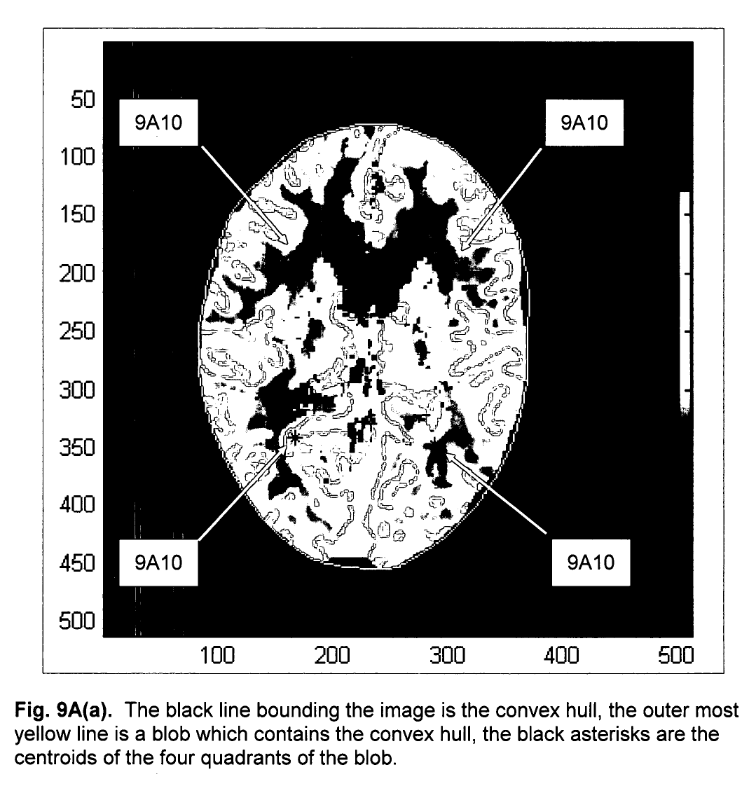

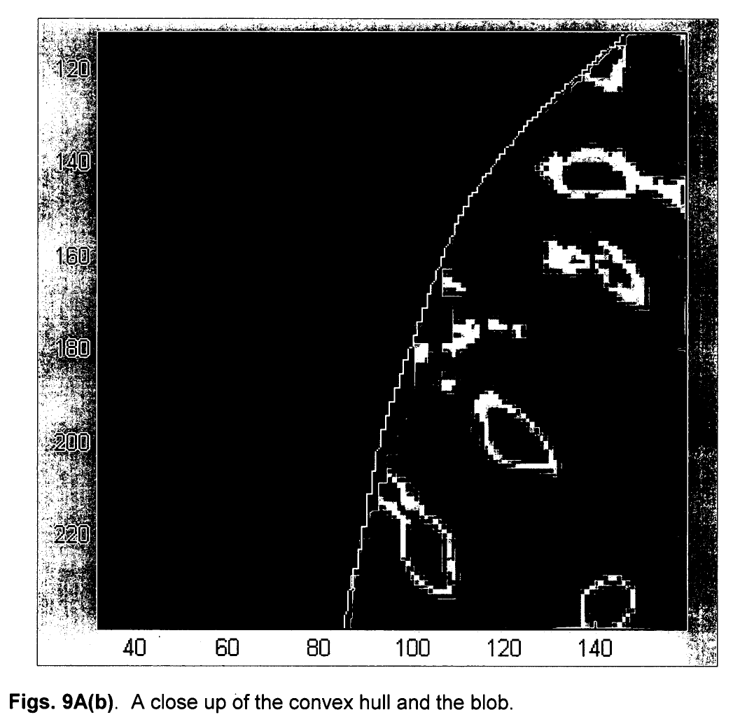

Thus, in exemplary embodiments of the present invention, the convex hull of a boundary can be obtained, by having the points on the convex hull being parameterized by theta. By linearly interpolating radii between two points on the convex hull, it is possible to obtain a “generic shape” that shares points with the convex hull, but smoothly varies from one point to the next (which may not be convex). This is shown, for example, in FIG. 9.

Such a function thus provides a way to determine if a point within a distance of Rₘₐₓ on the centroid is inside or outside of the region enclosed by the bounding function. This can be accomplished, for example, by calculating the angle and comparing the two distances obtained.

After a periodic bounding function has been obtained, Fourier Shape Descriptors of the bounding function as well as the centroids of any angular section of the object can be calculated. The difference in the shape descriptors for the generic shape and the bounding function can then, for example, determine the stopping point of the rays and therefore the shape of the convex hull.

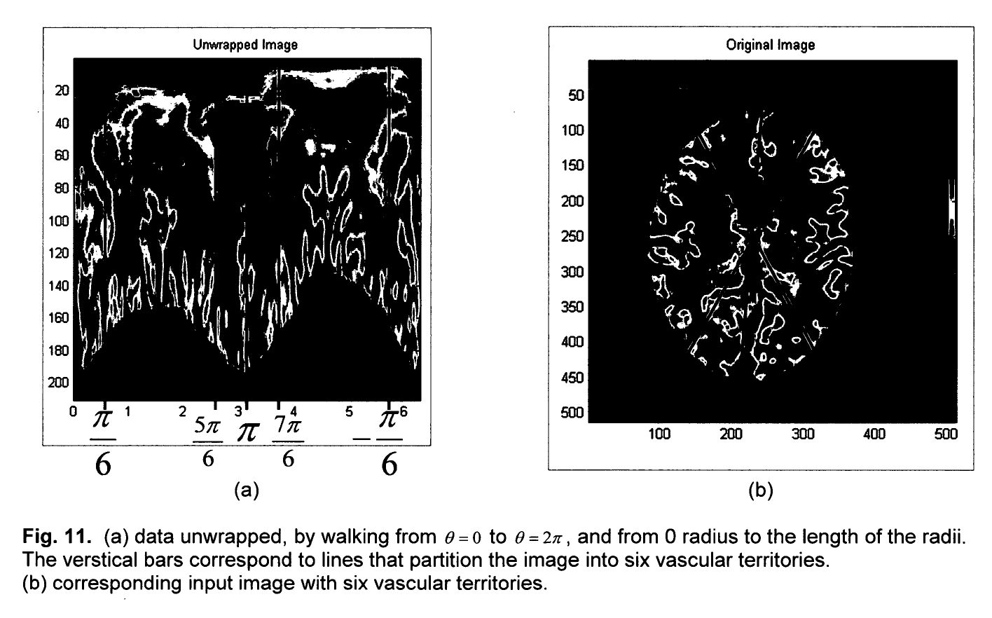

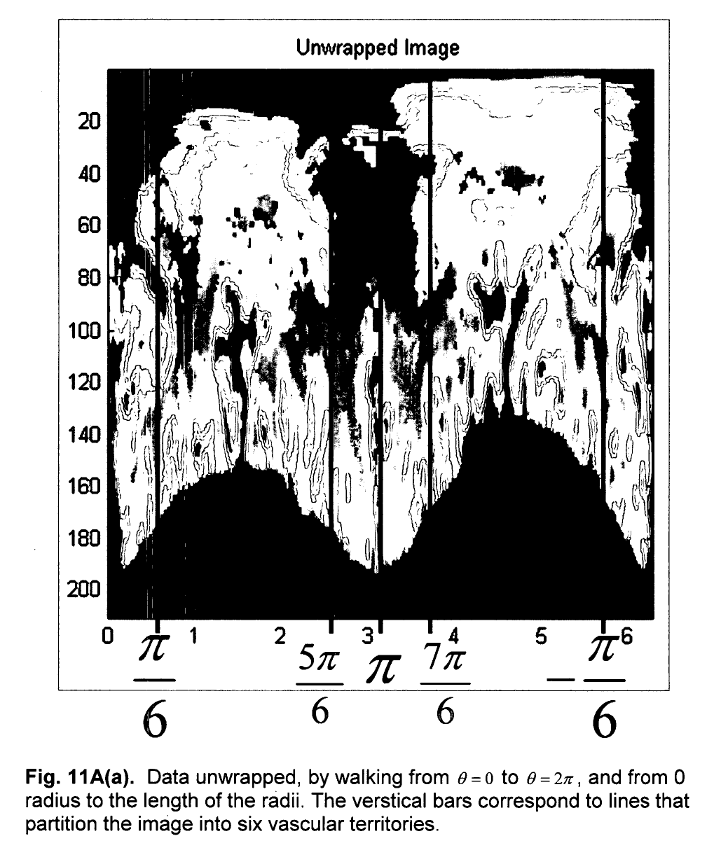

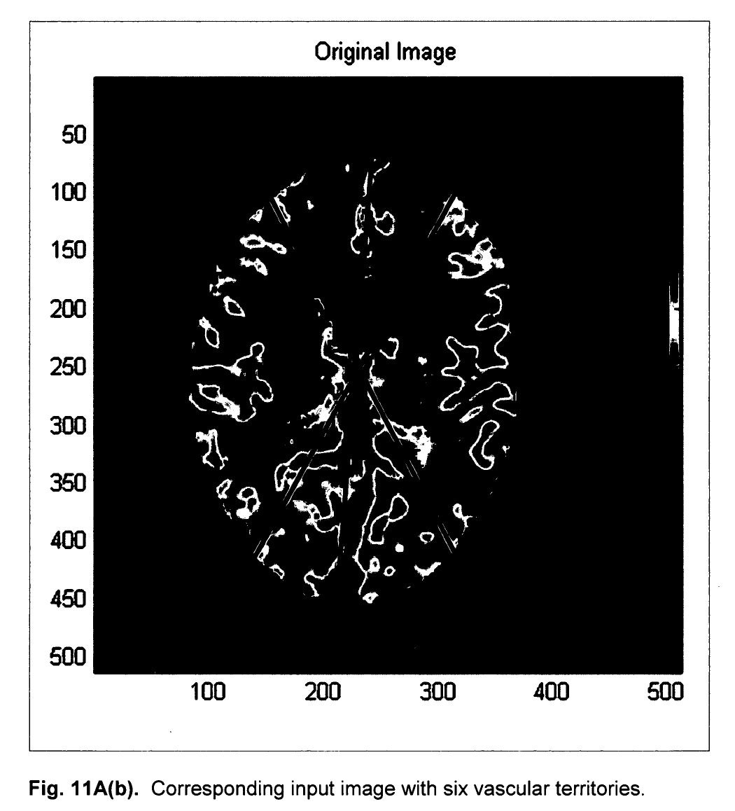

At this point the data can, for example be unwrapped by converting the convex image to a substantially rectangular image and each of the radii can be renormalized to equal length, to facilitate the comparison of features in the left and right halves of the image (an example of such unwrapping is shown in FIG. 11(a)).

Moreover, this procedure can be generalized to three dimensions, where the radius is a function of the solid angle, r(θ,φ),

$$0 \leq θ < 2 π , - \frac{π}{2} \leq ϕ \leq \frac{π}{2}$$

(duplicated points at −π/2,π/2 can removed later) extracted from these radii, and the “general shape” is constructed.

By iterating this technique with decreasing values of Rₘₐₓ, sets of radii can be constructed that can be combined to obtain the boundary of the object parameterized by arc length, thus extending the object to boundaries that are not a function of theta.

Although the above described method has applicability to virtually any substantially symmetrical object, the principles of the present invention are well suited for determining the presence of ischemia in the regions of the human body such as the brain. For example, it can be shown using the so called “maximum slope method” that CBF at any location in the brain may be determined by observing the maximum slope of C(t) at a particular location and dividing by the difference of Ca(t) at the input (e.g., anterior cerebral artery) and the output Cv(t) (e.g., superior sagittal sinus). This provides the following relationship:

$$( F / V ) = \frac{{( dC / dt )}_{tissue} ( {t}_{max\_slope} )}{( {C}_{a} ( {t}_{max\_slope} ) - {C}_{v} ( {t}_{max\_slope} ) )} \qquad ( 1 )$$

Thus, it can be seen from the relationship in equation 1 that the maximum slope for any tissue can be achieved at the same time when the input slope Cₐ(t) reaches its peak. Consequently, CBF, for example, can be calculated at any location by tracing the maximum slope and dividing it by the maximum intensity value at the anterior cerebral artery.

By observing a Cᵥ(t) curve to compute its maximum, CBF and CBV can be derived, for example, using the following relationships:

$$\begin{matrix}CBF ( any\_location ) = \frac{max\_slope ( any\_location )}{max\_value ( {C}_{v} ( t ) )} & ( 2 ) \\ CBV ( any\_location ) = \frac{max\_slope ( any\_location )}{max\_value ( {C}_{v} ( t ) )} & ( 3 )\end{matrix}$$

Furthermore, if Cₐ(t) and Cᵥ(t) curves are obtained independently and then are superimposed on one another, it is possible to asses different cardiac output. However, because the “relative” values of CBF and CBV are considered to be more reliable than the absolute values of these quantities, difference maps and relative difference maps can be calculated as described below.

In exemplary embodiments of the present invention, difference maps may be calculated by subtracting the pixel illumination value from one scanned hemisphere with those found on the contralateral hemisphere. For example, values for CBF can be calculated for both sides and compared. An ischemia score can be assigned on the pixel differential if significant CBF is detected.

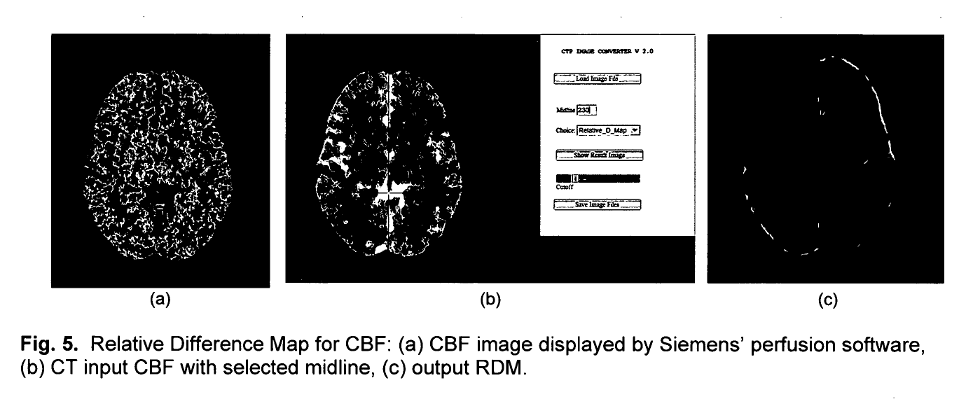

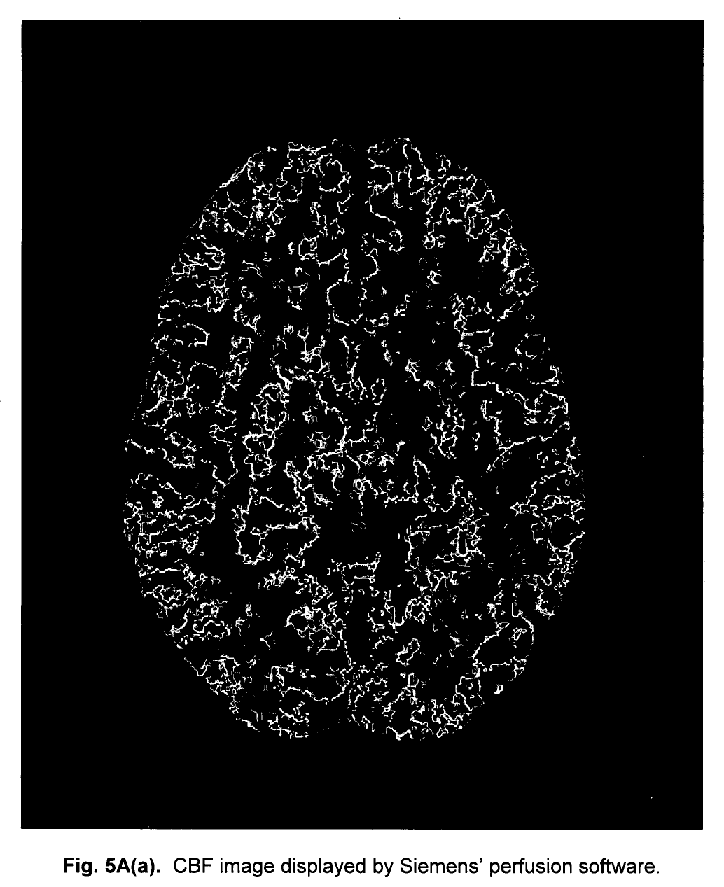

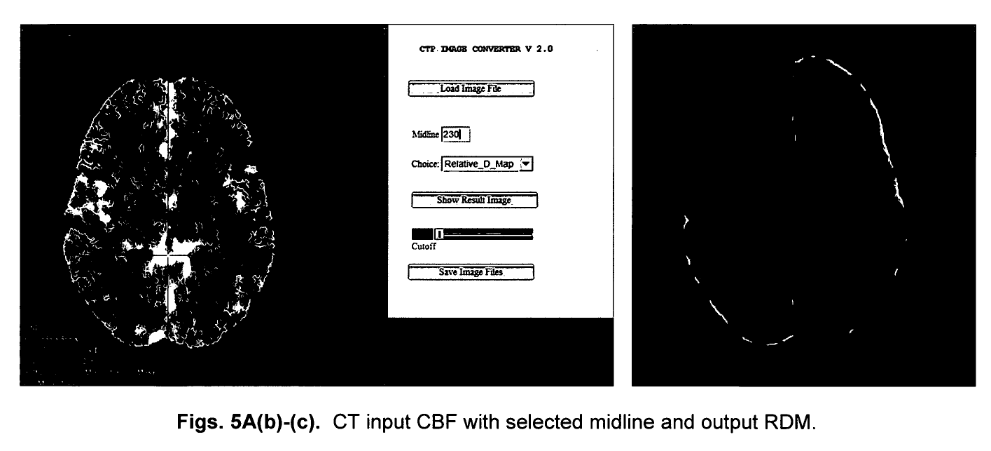

Relative difference maps can, for example, be obtained by comparing the pixel values of each hemisphere and computing the ratio of pixels with a lower CBF score to those with a higher one. Such a relative difference map can be displayed as a color differential map highlighting areas of ischemia or reduced blood flow for consideration by a medical specialist. Numerous examples of such maps are described below, such as, for example, FIGS. 5(c) and 5A(c).

The following provides a general list of conditions that can be employed, in exemplary embodiments of the present invention, to generate difference maps and relative difference maps for display to a user.

1. Difference Maps: DM-CBV, and DM-CBF.

(a) L2R DM (left to right difference map)
if 12r<0→display its absolute difference on the L side (pixel differential)
if 12r>→0display Black (zero intensity)

(b) R2L DM (right to left difference map)
if r21<0→we display its absolute difference on the R side (pixel differential)
if r21>→0 display Black (zero intensity)
   2. Relative Difference Maps: RDM-CVF, and RDM-CBF.

a) L2R DM
if 12r<0→compute the ratio of the absolute difference (pixel differential) divided by the intensity on the RIGHT (the good one) hand side, and display it on the LEFT hand side
if 12r>=0→display Black (zero intensity)

(b) R2L DM
if r21<0→compute the ratio of the absolute difference (pixel differential) divided by the intensity on the
   LEFT (the good one) hand side, and display it on the RIGHT hand side
if r21>=0→display Black (zero intensity)
   3. Relative Maps: RM-CVF, and RM-CBF. In relative maps the intensity on the BAD side (the one with the lower CBF value) is related to the intensity on the GOOD side (the one with the higher CBF value); this can provide the “intervals” for normalized relative values.

a) L2R DM
if 12r<0→compute ratio of intensity on the LEFT hand side (the bad one) divided by the intensity on the
   RIGHT (the good one) hand side, and display it on the LEFT hand side
if 12r>=0→display Black(zero intensity)

(b) R2L DM
if r21<0→compute ratio of intensity on the RIGHT (the bad one) divided by the intensity on the
LEFT (the good one) hand side, and display on the RIGHT hand side
if r21>=0→display Black(zero intensity)

For TTP, the reverse can be done:

1. Difference Maps: DM-TTP.

(a) L2R DM
if 12r>0→display its absolute difference on the L side (pixel differential)
if 12r<=0→display Black(zero intensity)

(b) R2L DM
if r21>0→display its absolute difference on the R side (pixel differential)
if r21<=0→display Black(zero intensity)
   2. Relative Difference Maps: RDM-TTP.

a) L2R DM
if 12r>0→compute the ratio of the absolute difference (pixel differential) divided by the intensity on the LEFT hand side (the bad one) hand side, and display it on the LEFT hand side, too
if 12r>=0→display Black (zero intensity)

(b) R2L DM
if r21>0→we compute the ratio of the absolute difference (pixel differential) divided by the intensity on the RIGHT (the bad one) hand side, and display it on the RIGHT hand side
if r21<=0→display Black (zero intensity)
   3. Relative Maps: RM-TTP. Compute the ratio of the “good side” the opposite to the “bad one

a) L2R DM
if 12r>0→compute ratio of intensity on the RIGHT hand side (the good one) divided by the intensity on the
LEFT (the bad one) hand side, and display it on the LEFT hand side
if 12r<=→display Black(zero intensity)

(b) R2L DM
if r21>0→we compute ratio of intensity on the LEFT (the good one) divided by the intensity on the
RIGHT (the bad one) hand side, and display on the RIGHT hand side
if r21<=→display Black(zero intensity)

In operation, using the above guidelines, an exemplary CT system can obtain a number of CT images to create the grayscale CBF image of a human brain, as shown, for example in FIGS. 5(b) and 5A(b). This image can be constructed, for example, using an exemplary CT system and known CT perfusion software such as, for example, that provided by Siemens. Such a grayscale image can, for example, be transformed into a predetermined binary format such, for example, as an eight bit digital word (byte) so that the scales in the original image can be normalized to range from zero to two hundred fifty five, which can be used to assign colors to the image. This is shown by the color image of FIGS. 5(a) and 5A(a). Although eight bit words are suitable for some embodiments of the present invention, it will be understood that in other exemplary embodiments words of a different size can be used to obtain images with a greater or lesser degree of precision, as may be desired.

Next, for example, an axis of symmetry can then be estimated (or computed) as a straight line drawn along the anterior-posterior axis through the septum pelucidum to equally divide the brain image shown in FIGS. 5(a)-(b) into two substantially symmetric hemispheres (right and left). This operation can, for example, be performed automatically in accordance with the present invention by comparison software, or can be selected manually by a medical practitioner performing the imaging.

Next, to quantify the symmetry of the scanned image, the comparison software can perform a statistical discrepancy test to determine the difference between the observed and expected cumulative frequencies between the data points acquired from the symmetric hemispheres. One such test suitable for this operation is the Kolmogorov-Smirnov test which is a non-parametric statistic test that does not require the acquired data points to be normally distributed as is the case, for example, in Gaussian based methods. Persons skilled in the art will recognize that other statistical tests may be used for this analysis. This test is based on the empirical distribution function, as defined in Equation 4, given N ordered data points Y₁, Y₂, . . . ,Y_{N}
EN=n(i)/N (4)
where n(i) is the number of points less than Yⁱ and the Yⁱ are ordered from smallest to largest value. This step function increases by 1/N at the value of each ordered data point. Using this formula, statistically significant differences between the two populations can be determined. This is preferably accomplished in accordance with the principles of the present invention by scanning each hemisphere into a number of overlapping symmetric sections or “windows” which then, for example, are compared against one another (from opposite hemispheres) to determine the absolute difference between the two. The size of the windows may vary depending on the size of the converted digital word or may be adapted to achieve a particular diagnostic goal. In one embodiment as represented by the algorithms described herein, a nine by nine pixel window can be used. It has been found that such a window provides good resolution as compared to the noise generated by small numbers of anomalous pixels. However, other window sizes can also be used as may be desired.

To determine asymmetries between the areas covered by the windows, the average intensities of pixels in one window from one hemisphere can be, for example, subtracted from those of the contralateral hemisphere, and the absolute difference can be divided by the intensity value on the side where CBF reading is relatively larger and higher (the “relatively normal hemisphere”). The result can be displayed on the side where the reading of the mirrored window is smaller (in the case of CBF and CBV parameters) or larger (in the case of TTP parameters) (the “relatively abnormal hemisphere”) to display the score for the relative difference map. A relative difference map of the image depicted in FIGS. 5(b) and 5A(b), constructed in accordance with the present invention is shown in FIGS. 5(c) and 5A(c).

Further analysis of the relative difference map shown in FIG. 5(c) can be performed, for example, by subdividing each hemisphere into six major cerebrovascular territories and calculating statistical difference charts, such as histograms, to illustrate (an subsequently analyze) the degree of difference between the two hemispheres. The degree of difference in each of the territories will be indicative of the type and degree of problem the patient is experiencing. As the peak of the curve moves further to the right, representing a greater degree asymmetry, the severity of the problem increases. For example, as shown in the histogram of FIG. 2, the “Normal Pt.” curve represents an expected distribution of relative difference values in a region of a brain that shows substantially normal blood flow distribution. Moving to the right in FIG. 2, the “Increased Vasospasm Risk” curve represents an expected distribution of relative difference values in a region of a brain that shows a blood flow distribution indicating an increased risk of vasospasm. Moving still further to the right, the “Vasospasm” curve represents an expected distribution of relative difference values in a region in a brain that shows a blood flow distribution indicating an existing vasospasm. Finally, at the far right, the curve labelled “Stroke” represents an expected distribution of relative difference values in a region in a brain that shows a blood flow distribution indicating a patient with an existing infarct.

Thus, in exemplary embodiments of the present invention, brain asymmetry can be quantified and analyzed, and used as a diagnostic tool to recognize or predict brain disease. By comparing successive histograms as shown in FIG. 2, for example, a medical practitioner can diagnose a slight brain condition that normally may go unnoticed, diagnose an existing brain disease with certainty, or by monitoring the progress of the peak of the curves on the histogram, recognize a trend or a degenerating state. This presents an advantage over existing techniques that merely display an image of the brain with color perfusion parameters indicative of blood flow that have to be manually compared and diagnosed. The quantification offered by the present invention can, for example, be used to supplement existing diagnostic techniques.

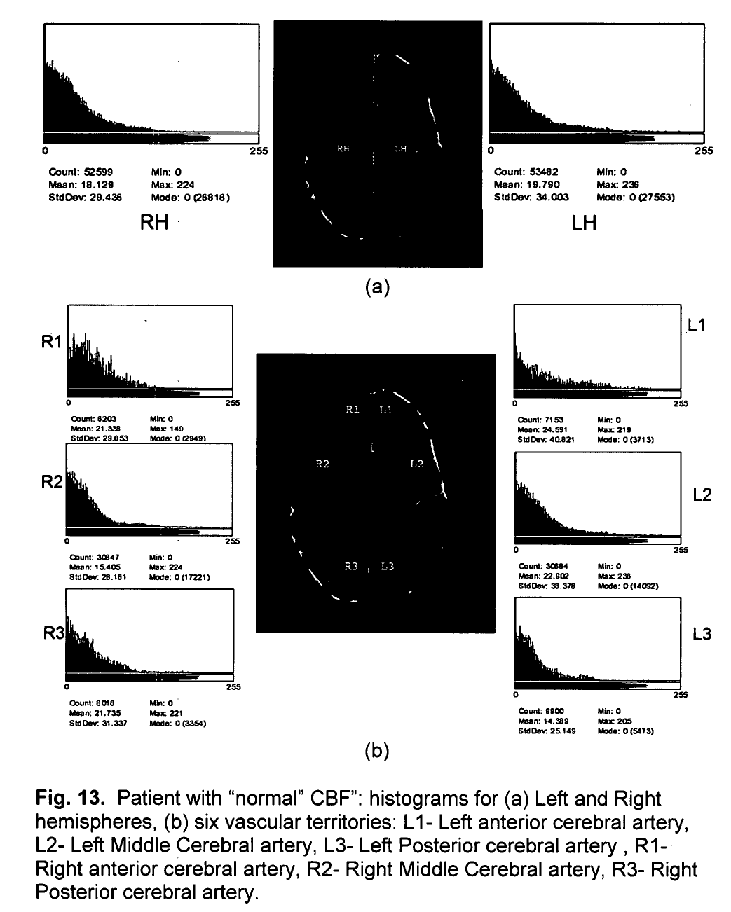

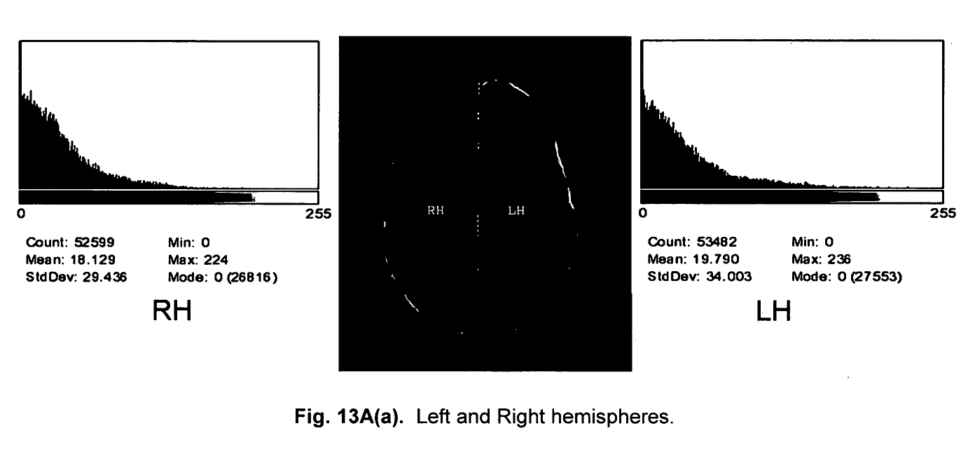

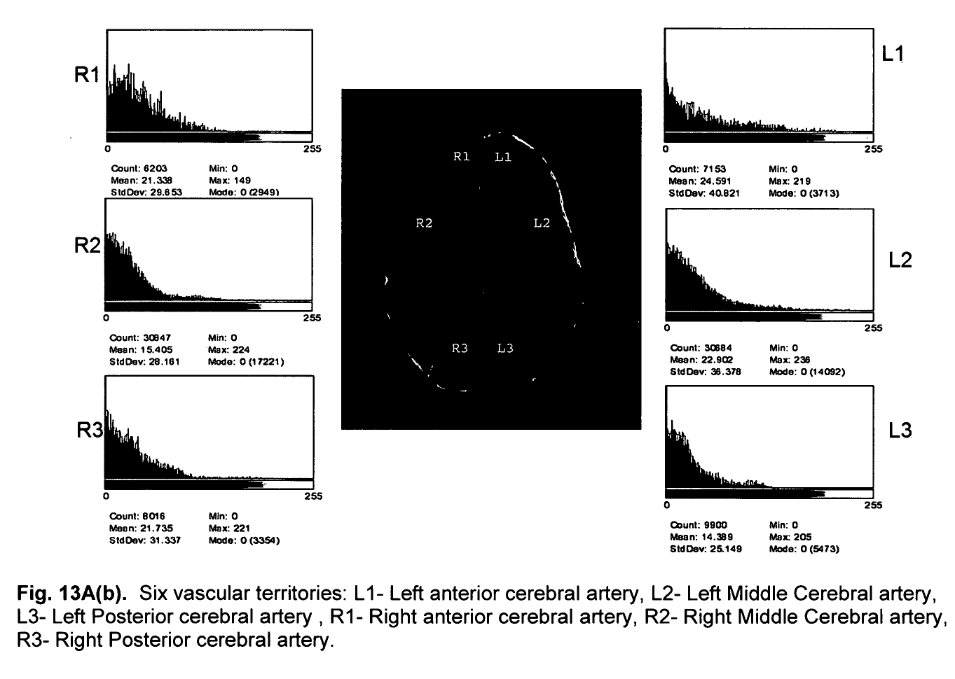

Examples of relative difference maps and histograms generated in accordance with the present invention are shown in the color images of FIGS. 13-15. FIG. 13(a) (it is understood that as many of the figures are presented in this application in both a smaller size and a larger size, a reference to the smaller sized version, as in in this case, for FIG. 13(a), also includes a reference to the larger size version of the same image, FIG. 13A(a). So as not to overburden the reader, this fact will not be repeated each time a reference to a figure is made, it being undersood that there are often two version of the figures herein) illustrates a relative difference map in the center with histograms RH and LH comparing each brain hemisphere. The patient from whom this data was obtained was a male in his early forties with a Hunt and Hess grade 2 SAH. Cerebral angiography disclosed a large (2 cm) MCA aneurysm, which involved the lenticulostriates. The patient's clinical course was unremarkable from a neurological standpoint, with no episodes of CVS detected by daily Transcranial Doppler sonography (TCD), cerebral angiography, or routine neurological examination. On SAH Day 5, the patient underwent a CTP scan, data from which was processed according to the present invention. The relative difference maps and histograms generated from the scan data are depicted in FIGS. 13(a)-13(b). FIG. 13(b) shows how six vascular cranial territories were assigned to the relative difference map in the center, with L1 representing the area including the left anterior cerebral artery, L2—representing the area including the left middle cerebral artery, L3—representing the area including the left posterior cerebral artery, R1—representing the area including the right anterior cerebral artery, R2—representing the area including the right middle cerebral artery, and R3—representing the area including the right posterior cerebral artery (although it is understood the these regions are exemplary, and may be rearranged or changed to serve different diagnostic goals). As can be seen from the regional histograms in FIG. 13(b), the various vascular territories demonstrate a relatively minimal deviation of the curve from zero in all territories, indicating relatively normal levels of perfusion throughout the brain.

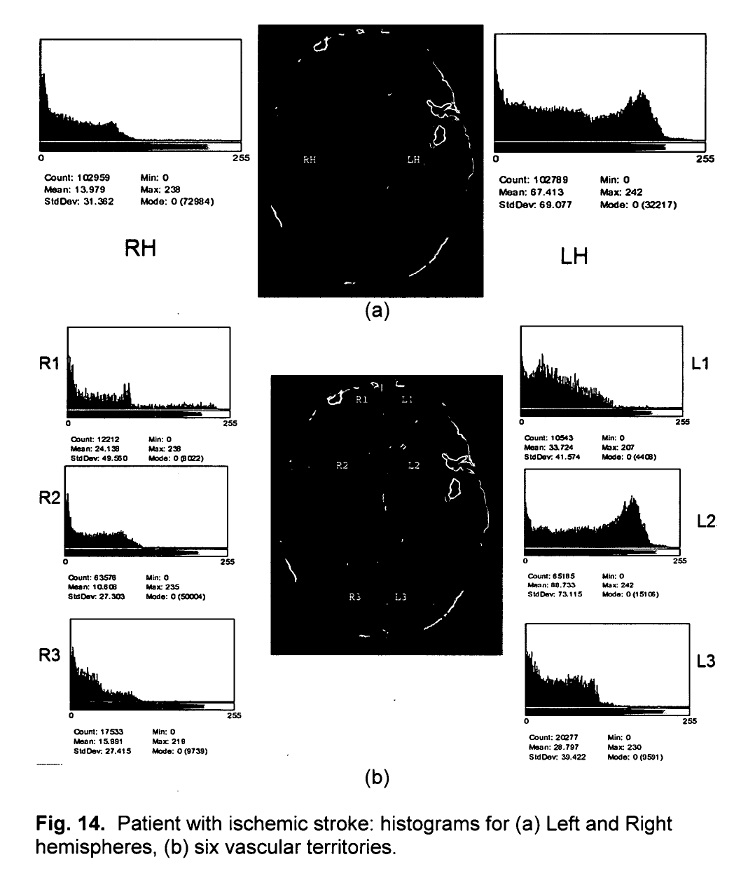

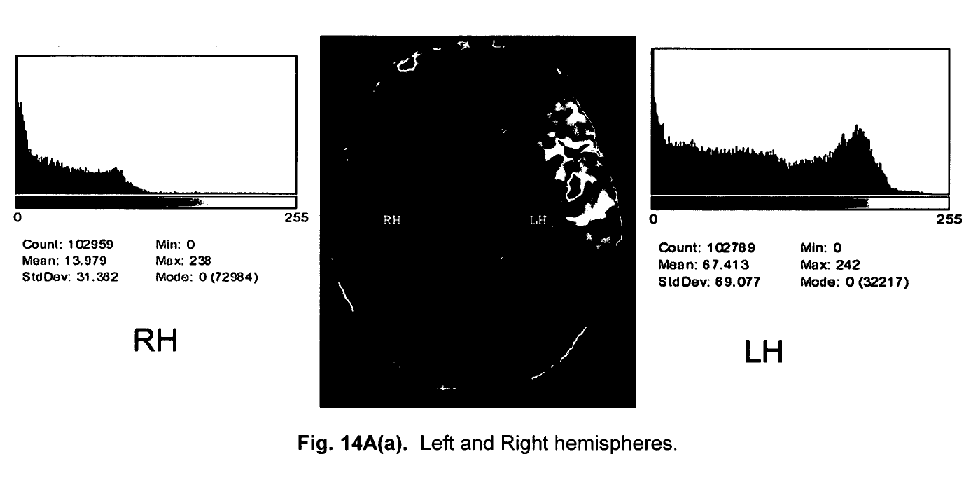

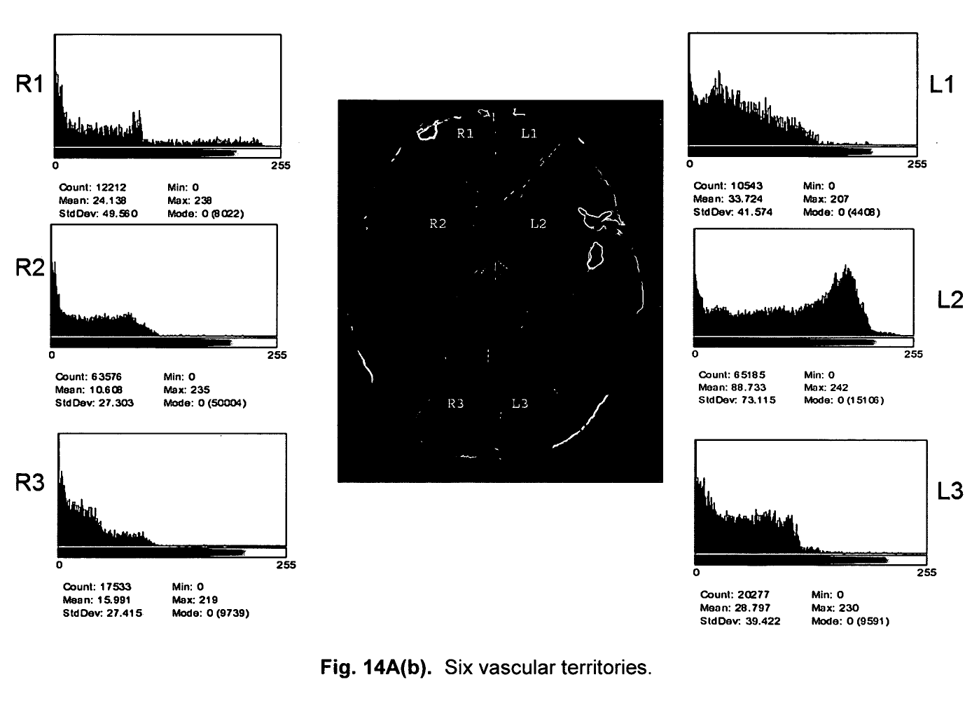

The patient from whom the data shown in FIGS. 14(a)-(b) was generated was a female in her late seventies who presented with symptoms consistent with left MCA infarction. The right side of the relative difference map in FIG. 14(a) (left side of the brain) clearly demonstrates a large wedge shaped region of severe hypoperfusion in the MCA territory which is consistent with an acute proximal MCA occlusion. When this image has been processed using the methods described herein, a clear peak on the far right is seen in the LH histogram representing the left MCA territory. This is consistent with the theoretical “Stroke” curve as shown in FIG. 2. The histograms for other vascular territories (L1 and L3 and R1-R3) in FIG. 14(b) have significantly smaller means (i.e., 33.724 for L1 and 28-797 for (3)), and many appear relatively “normal.”

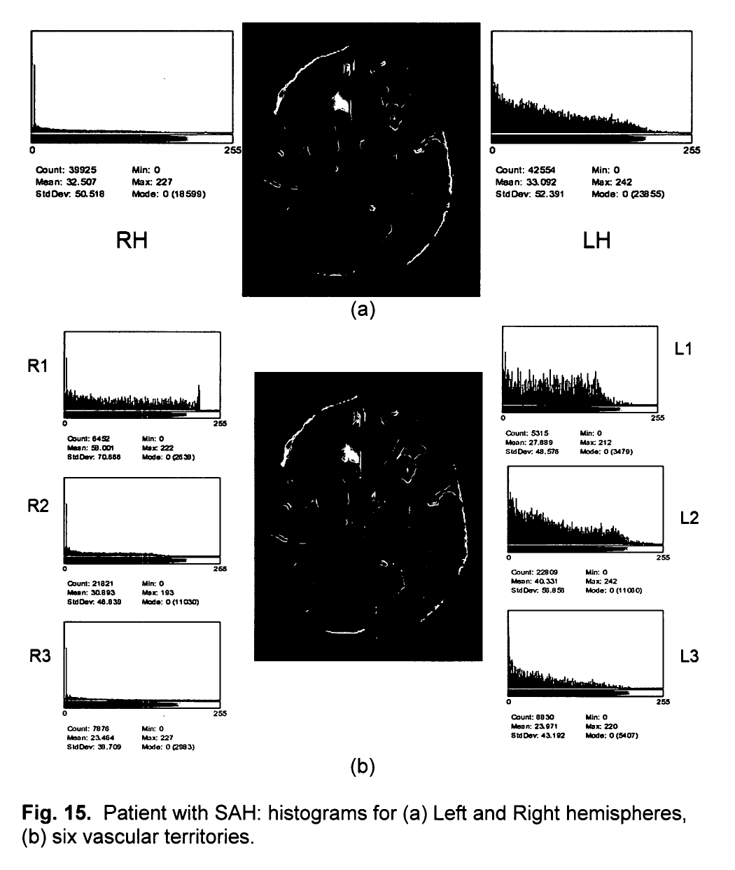

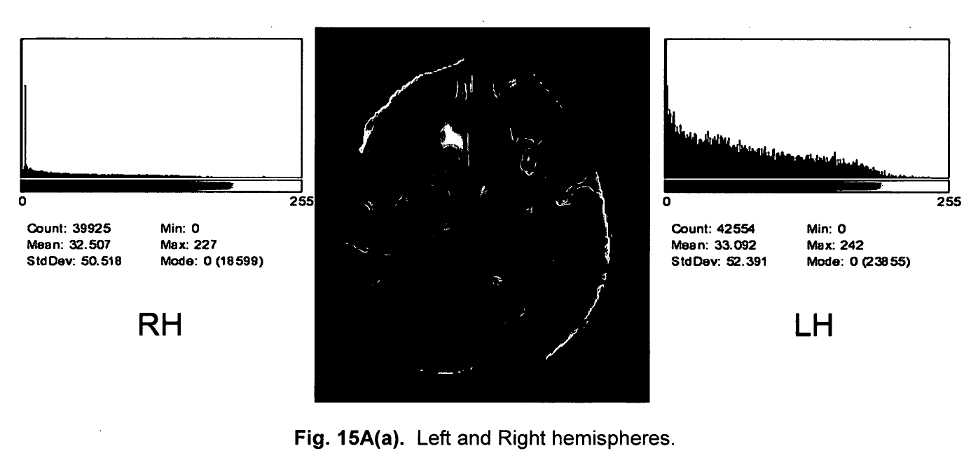

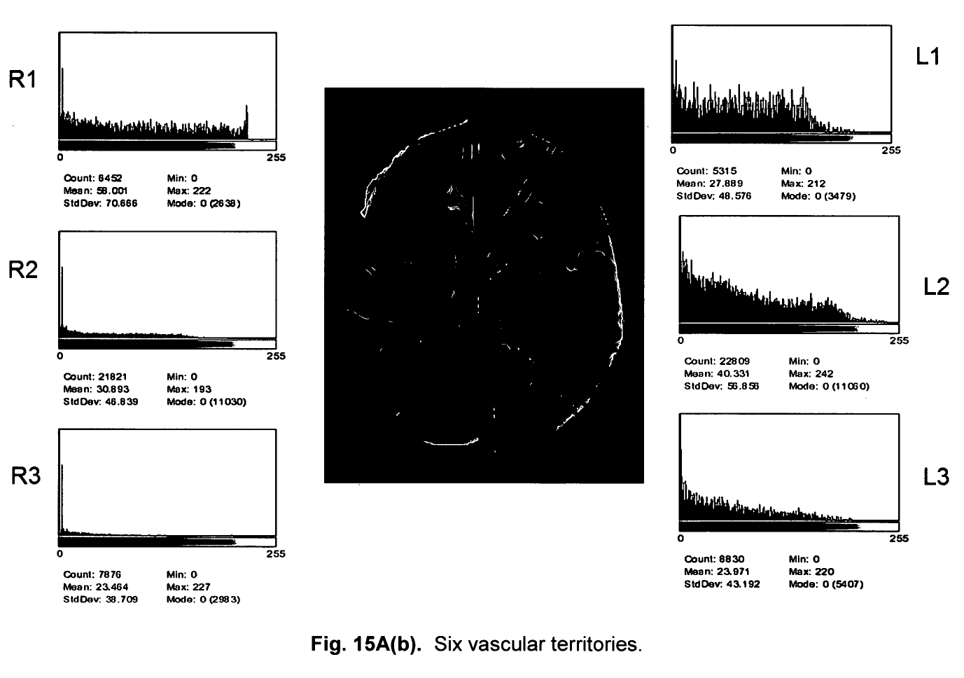

As another example, the patient for whom the data shown in FIGS. 15(a)-(b) was generated was a woman in her mid thirties who was presented with Hunt and Hess grade 4 SAH. Cerebral angiography disclosed a 4 mm×3 mm right anterior choroidal artery aneurysm. Her neurological examination improved significantly postoperatively. Where, on SAH day 5, she experienced an acute decline in mental status, neurological examination demonstrated no focal neurological deficit. The patient subsequently developed bilateral arm weakness and was taken for angiography, which revealed a severe vasospasm of the right and left MCA and right ACA. This spasm was treated with angioplasty, with significant clinical improvement. Unfortunately however, a follow-up MRI two months later demonstrated an old cerebral infarction in the right frontal lobe. The relative difference map of FIG. 15(b) thus demonstrates significant regions of side-to-side asymmetry in the Left MCA and Right ACA territories, consistent with the results seen at angiography. The histograms of these regions (L1-L3 and R1-R3), while not as striking as those seen for Patient 2 (FIGS. 14(a) and (b)), nevertheless demonstrate significant increases in frequency of significantly mismatched pixels (e.g., shift of the curve to the right for region L1).

In exemplary embodiments of the present invention, the methods and systems described herein for quantifying symmetrical portions of an image may be used for purposes other than assisting in the diagnosis of a given patient based on an image. For example, the methods may be applied to compare a patient's image with prior images from that patient to observe progress over time, or to create a medical history for the patient. Also, such methods can be applied to train medical students in reading radiological images or to assess a physician's diagnostic abilities. Or, for example, method of the present invention may be similarly applied in areas other than medical imaging, provided that the image represents and captures a symmetrical body having characteristics expected to be symmetrically distributed about an axis.

The methods and systems described herein may be implemented in software, firmware, hardware, or any combination(s) of software, firmware, or hardware suitable for the purposes described herein. Software and other modules may reside on, for example, servers, workstations, personal computers, computerized tablets, PDAs, and other computer readable memory devices suitable for the purposes described herein. Software and other modules may be accessible, for example, via local memory, via a network, via a browser or other application in an ASP context, or via other means suitable for the purposes described herein. Data structures according to exemplary embodiments of the present invention, can comprise, for example, computer files, variables, programming arrays, programming structures, or any electronic information storage schemes or methods, or any combinations thereof, suitable for the purposes described herein. User interface elements described herein may comprise elements from graphical user interfaces, command line interfaces, and other interfaces suitable for the purposes described herein. Screenshots presented and described herein can, for example, be displayed differently as known in the art to input, access, change, manipulate, modify, alter, and work with information.

II. Quantification Method for Determining Previously Undetected Silent Infaracts on MR-Perfusion in Patients Following Carotid Endarterectomy

In accordance with a method according to an exemplary embodiment of the present invention, the post-operative Magnetic Resonance Perfusion (MRP) scans of patients undergoing CEA were evaluated using a novel image-analysis algorithm to identify and quantify the amount of cerebral blood flow (CBF) asymmetry in each hemisphere, and to determine if post-operative neurocognitive decline is associated with cerebral blood flow changes. The algorithm for analyzing post-processed MRP images quantifies the degree of relative difference between corresponding regions in the ipsilateral and contralateral hemispheres, and converts CBF values that are meaningless outside of the context of a given scan to a relative difference, which highlights side-to-side asymmetry. A Relative Difference Map (RDM) was constructed by comparing a small region of the scan to the corresponding region in the contralateral hemisphere, quantifying the degree of relative difference, and representing this quantity of relative difference in 2D and 3D. The amount of relative difference in both hemispheres and in six major vascular territories was analyzed to assess the degree of hypoperfusion in the regions. Thus, an approach that automatically identifies the asymmetry and quantifies the amount of asymmetry was implemented. The results show promise in the discovery of pathologic areas of MR perfusion that are invisible to human experts or highly variable among different experts. The method can, for example, automatically identify the focal points and anatomical regions (vascular territories); derive the axis of reflexional symmetry in each slice in volumetric MRP-CBF data; compute a RDM; and generate histograms in each vascular territory.

For the analysis of this data, the following assumptions were made: (1) In normal cases, the axial MRP images of the left and right hemispheres are, for the most part, structurally symmetric and comparable, and there should be no more than minor side-to-side differences in relative blood flow between the two hemispheres. (2) In abnormal cases, the left and right hemispheres are still structurally symmetric and comparable; but there is significant relative blood flow difference between the two hemispheres, that can be detected using MRP images.

An algorithm was developed that quantified the degree of relative difference between corresponding regions in the ipsilateral and contralateral hemispheres. A Relative Difference Map (RDM) representing this quantity in 2D and 3D was constructed. The amount of relative difference in both hemispheres and in 6 major vascular territories (left anterior cerebral artery (LACA), left middle cerebral artery (LMCA), left posterior cerebral artery (LPCA), right anterior cerebral artery (RACA), right middle cerebral artery (RMCA), right posterior cerebral artery (RPCA)), was analyzed. The method automatically identified the focal points and anatomical regions (vascular territories); derived the axis of reflexional symmetry; computed the RDM; and generated histograms in each vascular territory. The histogram analysis linked the quantified symmetry to an assessment of pathology.

The exemplary method used included the following steps:
1) From an input image, FIG. 1(a), the center point (centroid) of an anatomical region of interest was automatically computed, as shown in FIG. 1(b);
2) From the centroid, a Fourier Shape Descriptor (FDS) was computed using ray casting, where all the pixels inside the brain were parameterized by the angle from the major axis and distance away from the centroid (in polar coordinates), as shown in FIG. 1(b);
3) The axis of reflection symmetry was derived, as shown in FIG. 1(c);
4) The orientation of the mass was detected by computing the angle between major axis and the vertical coordinate, and re-orienting the brain into the upright position, if needed;
5) The rotated image was unwrapped and re-coordinated based on the parameters formulated in step (2). The RDM was computed using a 9 by 9 window difference calculation and only highlighting the blood deficiency area into the corresponding brain side, as shown in FIG. 1(d);
6) The unwrapped RDMs were summed up and averaged out across the slices, which generated the mean relative difference map that reflects the volumetric information of given scans;
7) The unwrapped RDM was divided into six vascular territories by roughly estimating the angles, as shown in FIG. 1(e). The map (FIG. 1(e)) was disjoined and the histograms in all six territories were computed to yield the histograms of FIG. 1(f); and
8) The histogram of the calculated RDM, was examined to quantify the asymmetry

Thus, FIGS. 1(a)-(f) depicts various processing steps of an exemplary RDM method, as follows: FIG. 1(a) input image, FIG. 1(b) the centroid and Fourier Shape Descriptor (FDS), FIG. 1(c) axis of reflectional symmetry of the brain, FIG. 1(d) unwrapped 2D RDM image from the FDS, FIG. 1(e) RDM with automatically computed six vascular territories: L1—Left anterior cerebral artery, L2—Left Middle Cerebral artery, L3—Left Posterior cerebral artery, R1—Right, and FIG. 1(f) histograms corresponding to the six vascular territories.

The resulting RDM can be subdivided automatically into the six (6) major cerebrovascular territories, and regional histograms can then be calculated for each territory. As noted, FIG. 2 represents a hypothetical distribution of histograms of regions in the RDMs computed for CBF. These curves represent the conceptual basis behind the exemplary RDM algorithm. As shown in FIG. 2, it is believed that in disease states, differences will cluster in affected territories, and will skew the regional histogram to the right in “problem areas.”

To illustrate the results of this study, two cases are presented, one “normal” and one “injured” patient, each processed by a method according to an exemplary embodiment of the present invention. A single slice in the MRP-CBF volume is shown.

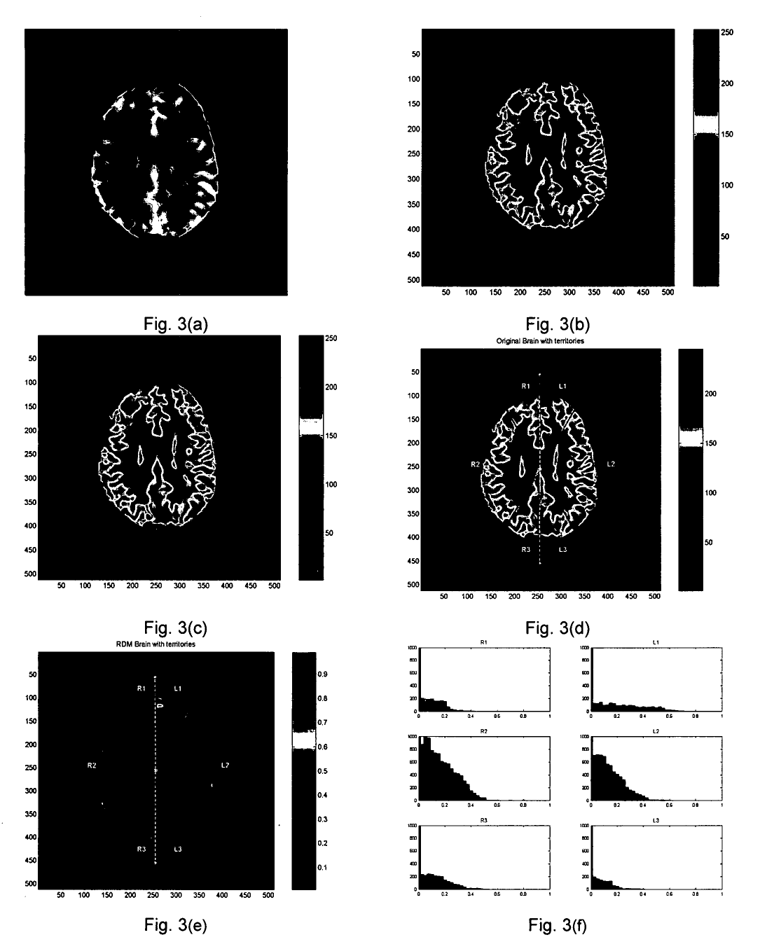

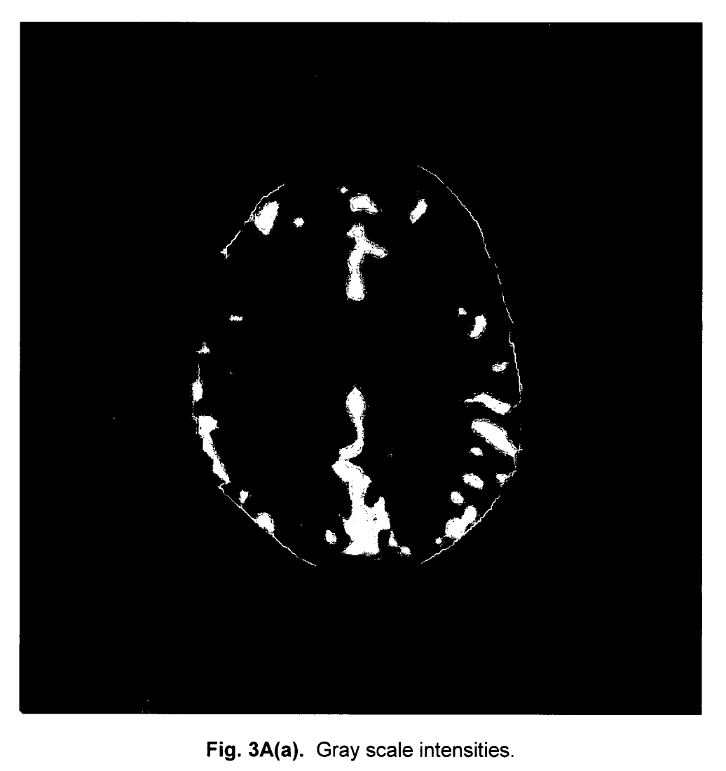

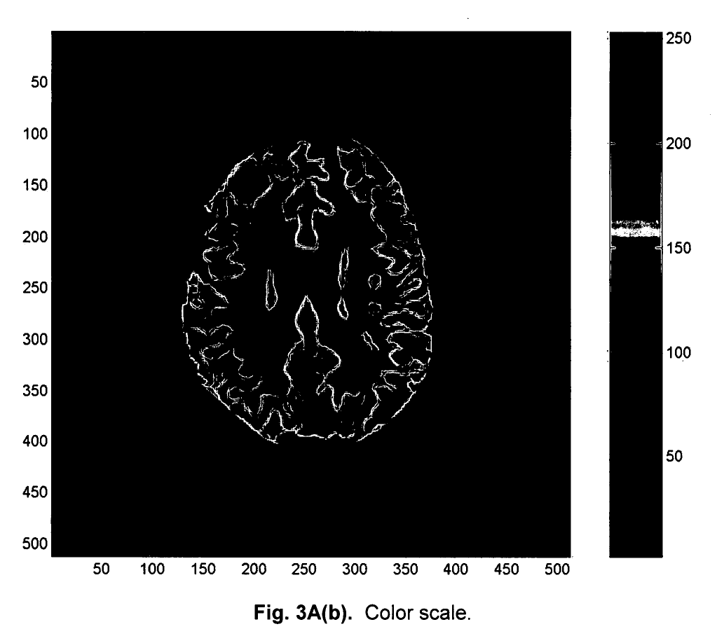

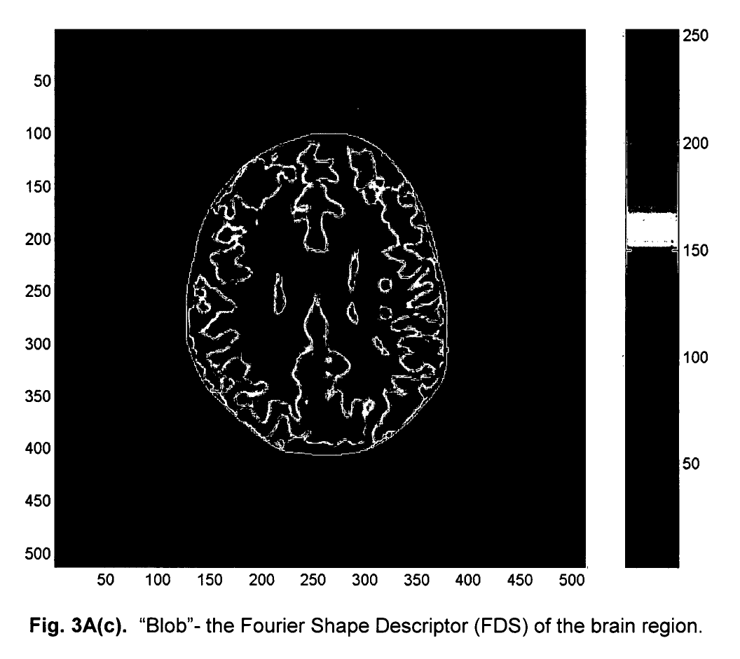

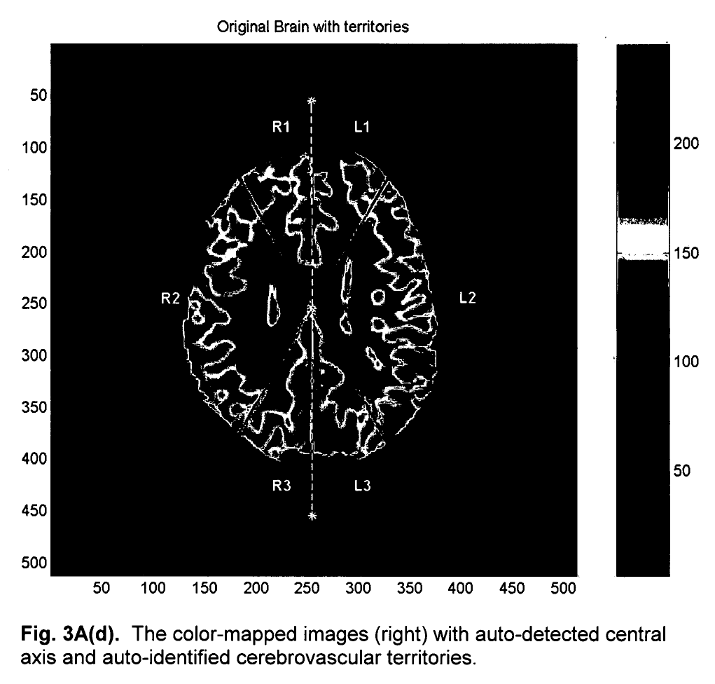

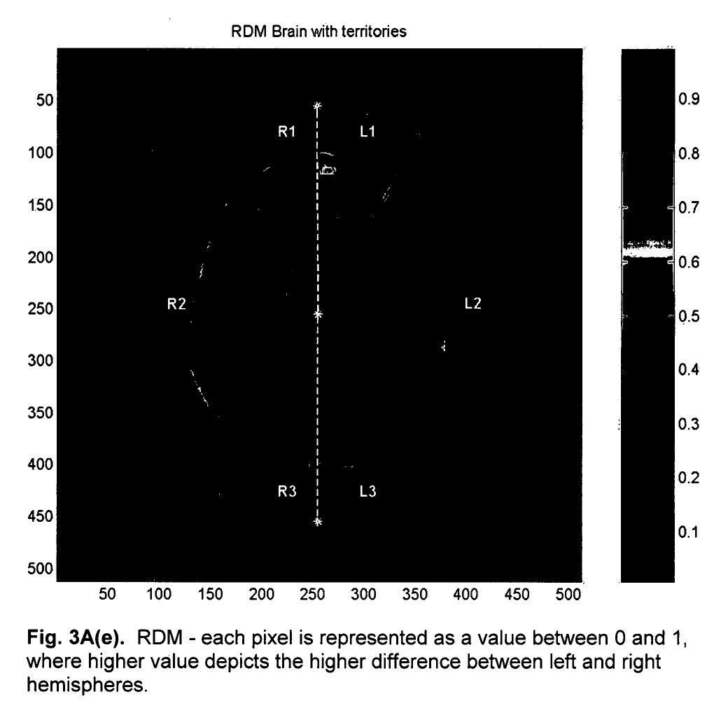

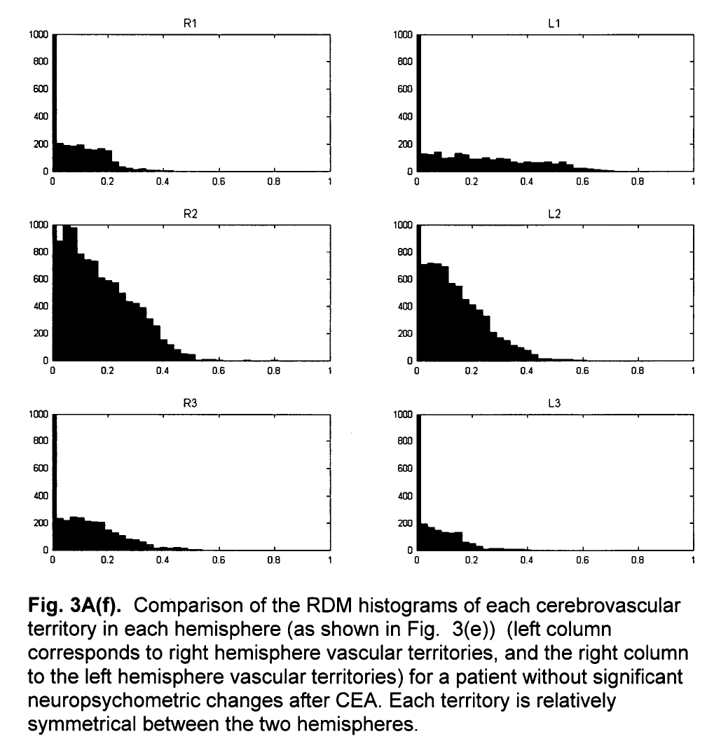

FIGS. 3(a)-(f) depict a normal patient: Input MR CBF slice in (a) gray scale intensities, (b) color scale; (c) “blob”—the Fourier Shape Descriptor (FDS) of the brain region; (d) the color-mapped images (right) with auto-detected central axis and auto-identified cerebrovascular territories; (e) RDM—each pixel is represented as a value between 0 and 1, where higher value depicts the higher difference between left and right hemispheres; (f) Comparison of the RDM histograms of each cerebrovascular territory in each hemisphere (in FIG. 3(e)) (left column corresponds to right hemisphere vascular territories, and the right column to the left hemisphere vascular territories) for a patient without significant neuropsychometric changes after CEA. Each territory is relatively symmetrical between the two hemispheres.

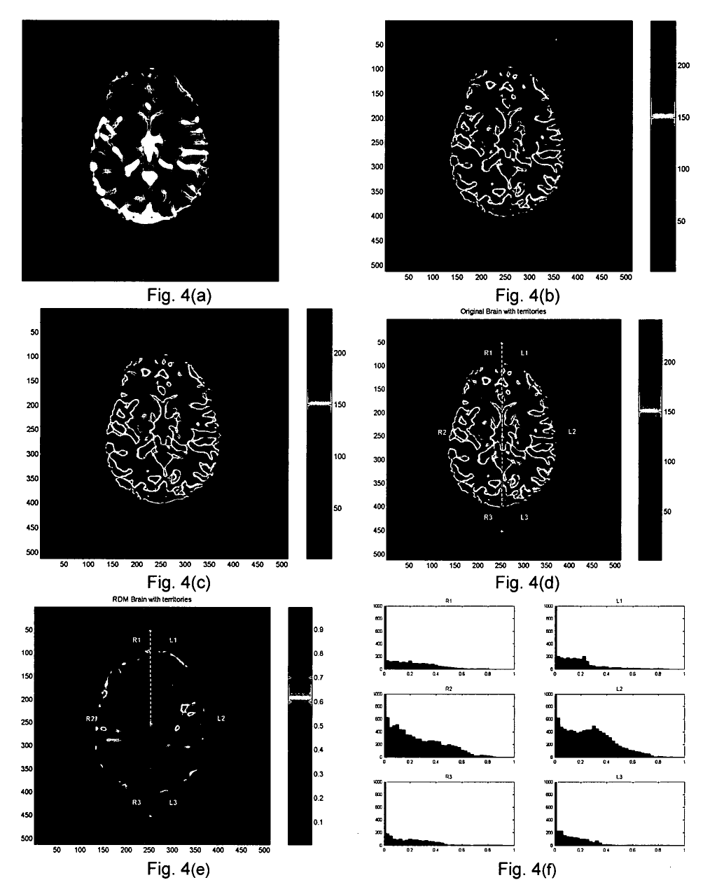

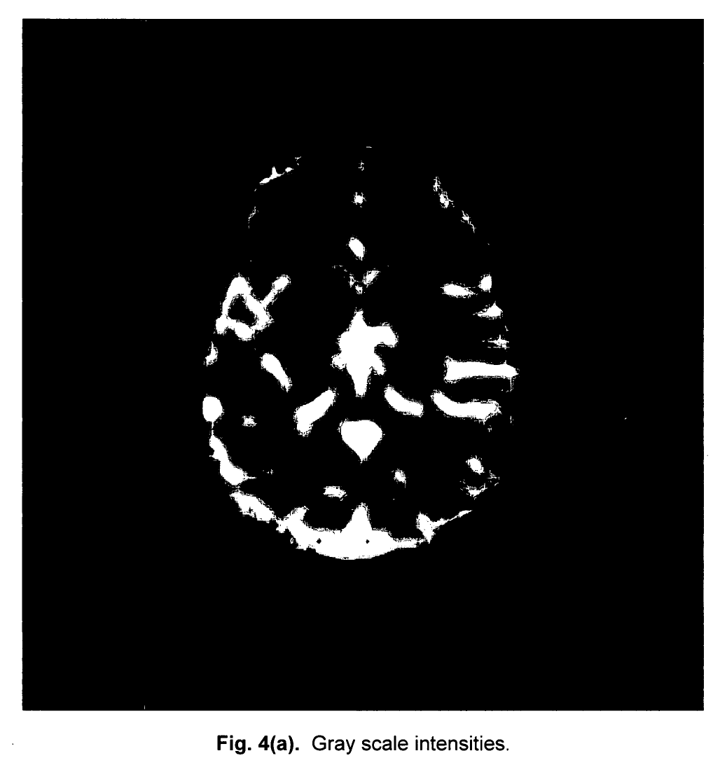

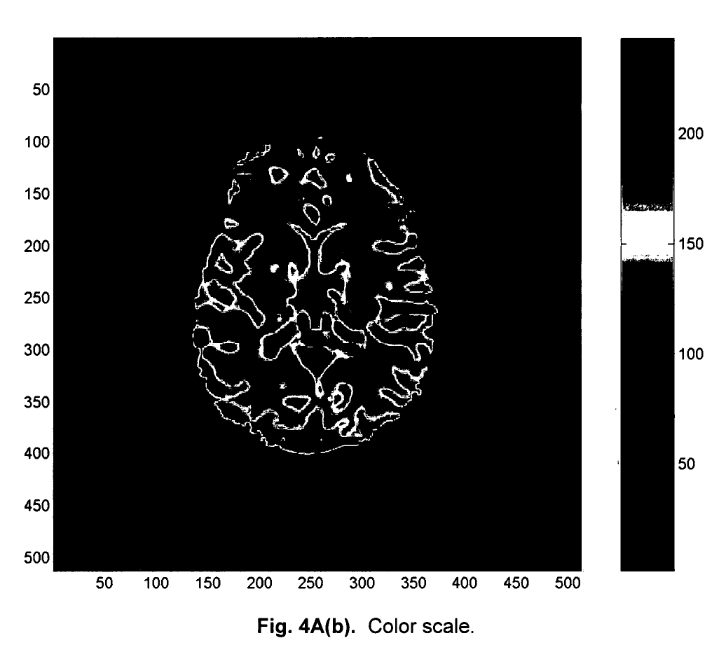

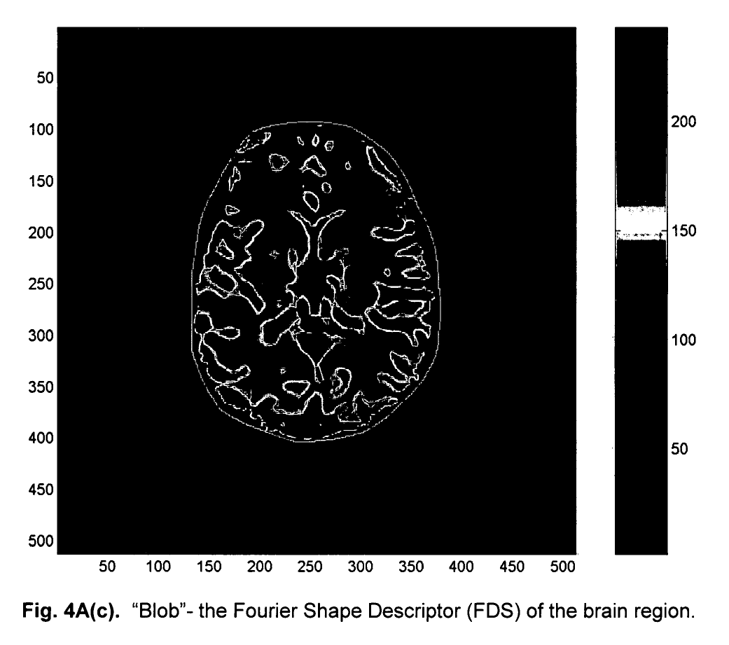

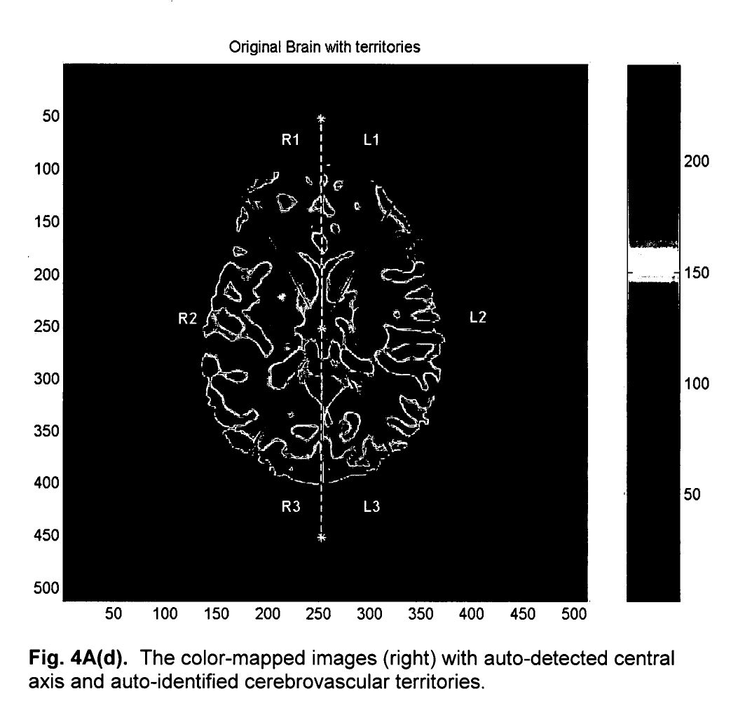

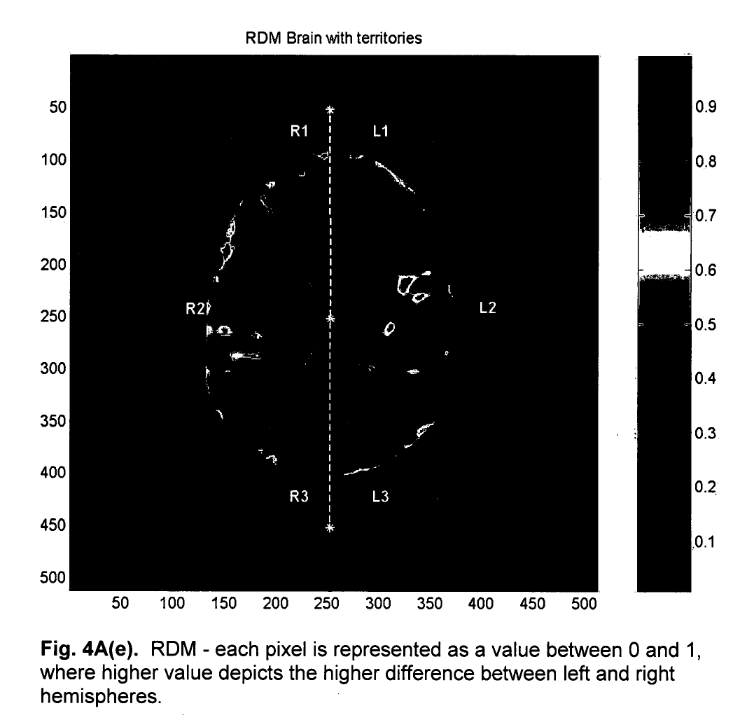

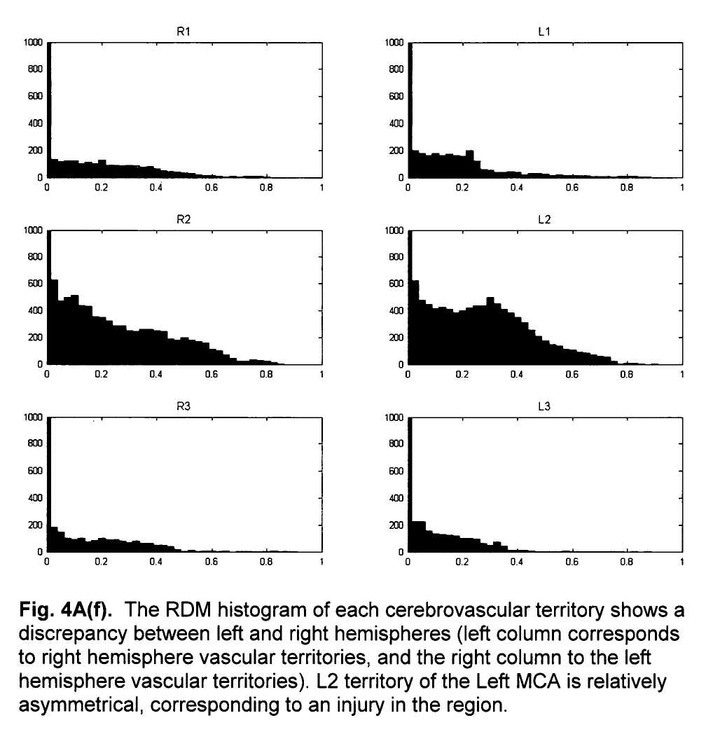

FIG. 4 depict the exemplary method applied to an “injured” patient with a problem in the left Middle Cerebral Artery (MCA), Input MR CBF slice in (a) gray scale intensities, (b) color scale; (c) “blob”—the Fourier Shape Descriptor (FDS) of the brain region; (d) the color-mapped images (right) with auto-detected central axis and auto-identified cerebrovascular territories; (e) RDM—each pixel is represented as a value between 0 and 1 (1 corresponding to dark red, dark blue to 0), where a higher value depicts the higher difference between left and right hemispheres; (f) The RDM histogram of each cerebrovascular territory shows a discrepancy between the left and right hemispheres (left column corresponds to right hemisphere vascular territories, and the right column to the left hemisphere vascular territories). The L2 territory of the Left MCA is seen as relatively asymmetrical, corresponding to an injury in the region.

24 MR perfusion scans were included in the analysis. These scans represent 22 CEAs and 2 spine control cases. Of the 22 CEAs performed, neurocognitive decline was demonstrated on post operative day 1 in 6 cases, yielding an neurocognitive “injury” rate of 27%. This incidence of neurocognitive decline is consistent with prior studies.

All of the 6 patients (100%) with detectable postoperative declines in neuropsychometric performance displayed asymmetric blood-flow changes on postoperative MRP scans when the algorithm was applied. The analysis revealed normal symmetric blood flow patterns in the 2 spine control patients and in 11 of 16 (69%) CEA patients who did not demonstrate neurocognitive decline. The remaining 5 “uninjured” CEA patients displayed asymmetric blood flow patterns, similar to those observed in the “injured” group. While these 5 cases may seem to represent possible inconsistencies in the algorithm, three of them can be explained by other factors. Two inconsistent cases are explained by asymmetric bifrontal artifarct, likely caused by magnetic interference by dental work. In both cases, the regions displaying blood flow asymmetry correspond to the location of the asymmetric artifact. A third inconsistent case can be explained by the occurrence of a pre-operative TIA. This TIA generated a DWI positive lesion in the superior-posterior left frontal lobe within 24-hours of the operation. The DWI positive region corresponds to the location of blood flow asymmetry detected by the exemplary algorithm. Although this patient did not demonstrate decreased neuropsychometric performance, the agreement between the DWI scan and the described findings validates the exemplary technique. The final two inconsistent case cannot be explained by asymmetric artifact or cerebral ischemia. Further analysis of the processed MRP scans in these case demonstrates that, when multiple slices are taken into consideration, blood flow appeared more globally symmetric that the result obtained from applying the algorithm to a single slice. When excluding MR perfusion scans with explained inconsistencies, 11 of 13 (85%) “uninjured” CEA patients displayed symmetric blood flow patterns.

The strong correlation between blood flow asymmetric, as detected by the exemplary method, and subtle declines in post-operative cerebral function, suggests that the algorithm is more sensitive than traditional evaluation of MR perfusion scans in detecting blood flow changes. This finding also suggests that hemodynamic alterations may play a role in decreased neuropsychometric performance following CEA.

Thus, conventional assessment of MRP scans was unable to demonstrate significant postoperative changes even in patients with postoperative cognitive deficits, whereas asymmetrical changes in MRP were detected by RDMs in patients who had evidence of cognitive deficits. Significant RDM differences with MRP scans between the two hemispheres were seen only in patients who had significant cognitive deficits. Thus, the disclosed methodology provides a better analysis of MRP parameters in patients with significant cognitive deficits.

III. Toward Objective Quantification of Perfusion-Weighted Computed Tomography in Subarachnoid Hemorrhage: Quantification of Symmetry and Automated Deloineation of Vascular Territories

Stroke is the third leading cause of death and disability in contemporary society. Subarachnoid hemorrhage (SAH) accounts for approximately 6 to 8% of all strokes and 22 to 25% of cerebrovascular deaths. Between 1 million and 12 million people in the United States harbor intracranial aneurysms, and the annual prevalence of aneurysmal SAH (aSAH) in this country is believed to be in excess of 30,000 persons. Despite considerable advances in the diagnosis and treatment of SAH, the outcome remains poor. The presence of subarachnoid blood is sufficient to produce severe luminal narrowing of cerebral arteries. This process is called cerebral vasospasm (CVS). However, the primary mechanism for vasospasm is uncertain, and therefore, any patient with SAH is considered to be at risk for developing CVS. Rapid diagnosis and treatment of CVS thus poses a true clinical dilemma and the lack of reliably predictive tests often leads to delayed intervention and irreversible neurological injury. Developing a non-invasive, reliable, and sensitive test that could risk stratify patients for CVS during the first days of their hospital admission into high, medium, and low risk populations would be of immense benefit for patients and could potentially decrease health care costs.

Perfusion-weighted computed tomography (CTP) is a relatively recent innovation that utilizes a series of axial head CT images to track the time course of signal from an administered bolus of intravenous contrast. These images are then processed using either deconvolution or maximum slope algorithms to extrapolate a numerical value for cerebral blood flow (CBF). While “bolus tracking” methods may provide accurate quantification of CBF under controlled conditions, variability in cardiac function, systemic blood pressure, and cerebrovascular tone often seen in the setting of acute SAH makes quantitative and qualitative assessment of these studies both difficult and potentially hazardous.

Although CTP has found some utility in the diagnosis and management of ischemic stroke, its potential use in the diagnosis and management of delayed CVS has not been investigated. Furthermore, since this imaging study is both fast and non-invasive, it is an ideal diagnostic test in this unstable patient population. Unfortunately, due to the inherent variability described above, there is no currently accepted, standardized method of interpreting these scans. Most commonly, scans are interpreted using the qualitative detection of gross side-to-side asymmetry of CBF, an approach that lends itself to misdiagnosis and potential failure to treat CVS. Recent work with CTP has focused on the development of methods to quantitatively analyze CTP images. Most of these approaches utilize the region of interest (ROI) method. In this approach, the clinician circles an ROI on the post-processed CTP image, and the mean CBF is compared to that of the corresponding ROI in the contralateral hemisphere to detect asymmetry. A growing body of data supports improved safety and efficacy of this approach in the setting of acute ischemic stroke, though data for SAH is less complete.

An algorithm according to the present invention was used for analyzing post-processed CTP images obtained from a standard perfusion CT software package (obtained from Siemens Medical Solutions). The method converts CBF values, which must be viewed as meaningless outside of the context of a given scan, to relative difference, which represents side-to-side asymmetry and is a meaningful value, inasmuch as it is specific to a given scan. The conversion was performed by comparing a small region of the scan to the corresponding region in the contralateral hemisphere, quantifying the degree of relative difference, and representing this quantity of relative difference in 2D and 3D in a Relative Difference Map (RDM). The amount of “relative difference” in both brain hemispheres and the 6 major vascular territories was analyzed and quantified to assess the degree of hypoperfusion in the regions. The method was automated and can provide a better and more stable analysis of the perfusion parameters of SAH patients.

Materials and Methods

Patient Population and Image Selection

Between 1996 and 2003, all patients presenting to New York Presbyterian Hospital following SAH resulting from the rupture of an angiographically confirmed intracranial aneurysm were prospectively enrolled in the SAH Outcomes Project (SHOP), an IRB approved project in which extensive clinical and long-term health outcomes data used were collected on all patients.

CTP studies were performed by the treating physicians as a part of routine management. During a 2-year period (July 2001 to July 2003), 122 CTP scans were performed in 85 patients in SHOP. DICOM images from these patients were collected retrospectively, processed by a neuroradiologist, and selected for this analysis. Unusable scans were excluded on the basis of having poor tissue enhancement curves. All images of SAH patients were taken from this cohort.

Based on the assumptions described below, patients with significant midline shift from focal hematoma, hydrocephalus, or focal cerebral edema were excluded.

Premises and Assumptions

For the analysis, the following assumptions were made: (1) In normal cases, the axial CT images of the left and right hemispheres are structurally symmetric and comparable, and there should be no more than minor side-to-side differences in relative blood flow between the two hemispheres; (2) In abnormal cases, the left and right hemispheres are still structurally symmetric and comparable, but a significant relative blood flow difference between the two hemispheres; this can be detected using CTP images.

The Relative Difference Map (RDM) as a Measure of Asymmetry

Original color DICOM images, as shown in FIG. 5(a) were transformed into an 8 bit binary format, where the color scales in the original images were normalized into a scale ranging from 0 to 255, as shown in FIG. 5(b). Using the stated assumptions, a straight line representing the image midline (an axis of symmetry) was drawn by hand along the anterior-posterior axis through the septum pelucidum to equally divide the brain into two symmetric hemispheres (see FIG. 5A(b) for detail). For quantification of symmetry, two 9 by 9 windows were constructed in both brain hemispheres that visit the opposite regions pixel-by-pixel in a scan-line fashion. Centered at a pixel location at its 9 by 9 window is compared to its sister window in the opposite hemisphere, around the constructed axis of symmetry. The Kolmogorov-Smimov test was applied to find the greatest statistical discrepancy between the observed and expected cumulative frequencies between two populations. The average intensities of pixels in the window from one hemisphere were subtracted from those of the contralateral hemisphere, and the absolute difference was divided by the intensity value on the side where CBF reading is relatively larger (“relatively normal hemisphere”). The result was displayed on the side where the reading of the mirrored window is smaller (“relatively abnormal hemisphere”) to display the score for relative difference map, FIG. 5(c). While the data in this figure utilizes CBF data, an identical method can be used to quantify the cerebral blood volume (CBV), and an inverse method can be used to quantify the time to peak (TTP) parameter.

The resulting RDM was then subdivided by hand into the 6 major cerebrovascular territories, and regional histograms are calculated for each territory. As noted, FIG. 2 represents a hypothetical distribution of histograms of regions in the RDMs computed for CBF. These curves represent the conceptual basis behind the exemplary algorithm. It is hypothesized that in disease states, “difference” will cluster in affected territories, and will skew the regional histogram to the right in “problem areas.”

Toward a Fully Automated System for Quantification of CTP

While the original method appeared promising, its dependence on a significant degree of user input limited its potential in that it was entirely dependent on subjective determination of the axis of symmetry and delineation of cerebrovascular territories. Thus, minor intra-observer variations could potentially cause significant alteration in the data provided by RDMs. As well, the method was very time consuming and not feasible to use in a clinical setting.

Thus, a methodology was developed by which RDMs could be generated with minimal user input. This presented a particular challenge in the SAH population as CTPs were obtained from brains of differing sizes and shapes, with varying degrees of minor distortion of cortical surface anatomy caused by extracerebral masses, specifically post-operative pneumocephalus, or small amounts of subarachnoid blood. While such blood and air can be segmented out and do not appear in the post-processed image, minor distortions of brain shape are very common in the SAH population and present a special difficulty in an attempt to design automated systems for assessing symmetry.

The developed method addressed this problem and can be roughly summarized in four key steps:

(1) Computation of axis of symmetry of an input CBF image and re-orientation of the image in upright position, if necessary;

(2) Computation of unwrapped image;

(3) Computation of RDM using the assigned axis of symmetry and 9 by 9 window difference calculation on the unwrapped image. Re-sampling of data back onto the original dataset; and

(4) Registration of six vascular territories using generic angles and computation of a histogram for each territory.

Automated Determination of the Axis of Symmetry

Computation of axis of symmetry in an irregular 2D image is a multi-step process which can be performed using the following processes: (a) computation of the convex hull of the anatomical region (i.e., an outline of a brain region); (b) computation of centroids from which the axis of symmetry is derived; (c) unwrapping of the image onto a rectangular stretcher with the axis of symmetry as a vertical midline, to potentially calibrate selection of the axis using underlying image information.

Computation of the Convex Hull

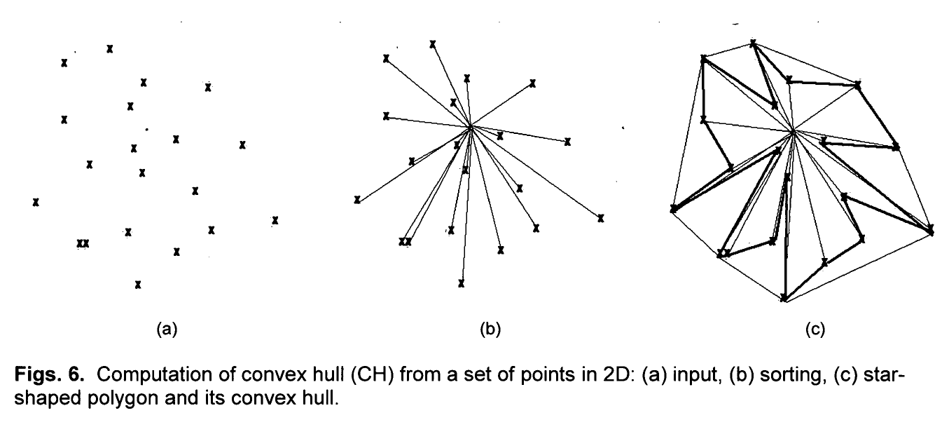

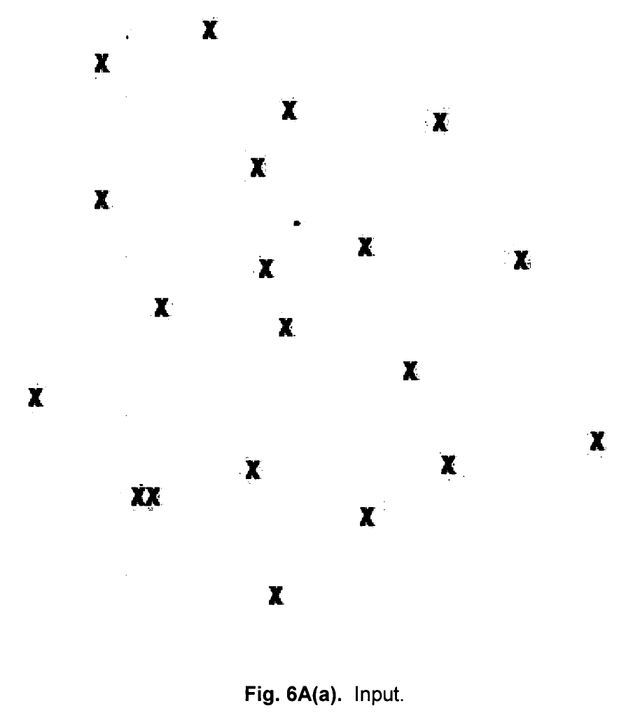

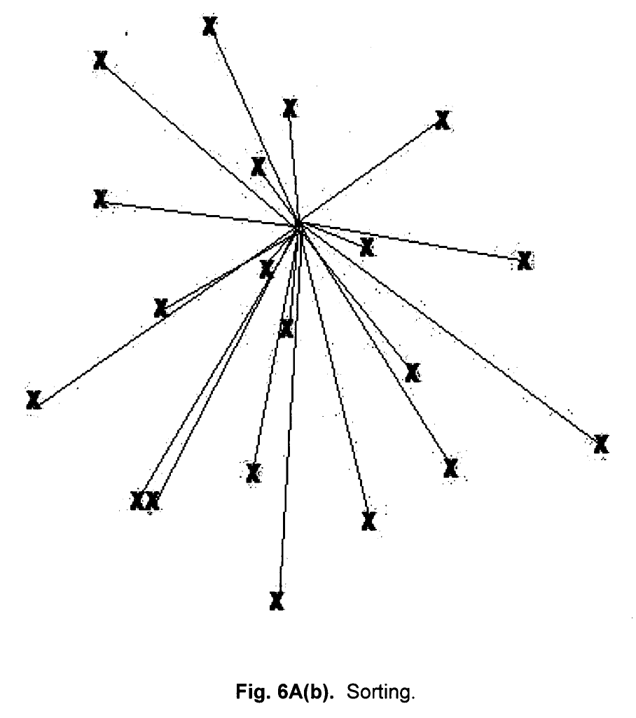

A 2D region is convex if a line segment between any two points in the region is inside that region. A convex hull of a 2D shape is the smallest convex region that fully encloses the shape. There are standard techniques to compute a convex hull for a set of points in 2D by first constructing a star-shaped polygon as is illustrated in FIG. 6. Because the images consisted of digital pixels, a method was devised that can sweep a 2D digital image from a centroid point by visiting each pixel and generating a star-shaped polygon that allows quick and easy computation of the convex hull.

Computation of Fourier Shape Descriptor Using Ray Casting: Determination of Bounding Functions of Theta

For a given 2D digital image, to obtain a boundary function for a region in the image described by points on the outer most edge of the region, rays can be cast from the centroid (cₓ,c_{y}) of the region outward at specific increasing angles θ. To obtain a valid sampling, theta was increased at each step by a

$$Δ θ < {sin}^{- 1} ( \frac{1}{2 {R}_{max}} ) .$$

As indicated in FIG. 7, some rays may pass through a singularity in the boundary, meaning that it is possible that a ray will pass between two boundary pixels that are diagonally connected. To prevent this the ray can be made to have a thickness on the order of the precision of the machine FIG. 8.

By recording the length of the ray at each angle, a description of a bounding function R(θ) of the region as a function of θ is obtained. However, this function may not have desirable properties; for example, it may not be convex. The convex hull of the boundary can thus be obtained with the points on the convex hull still being parameterized by theta. By linearly interpolating the radii between two points on the convex hull, it is possible to obtain a “blob” who which shares points which the convex hull, but smoothly varies from on point to the next. While it may not be absolutely convex, it can be “nearly”-convex, as shown in FIGS. 9.

Such a function does provide an easy means of determining if a point within a distance of Rₘₐₓ of the centroid is either inside or outside of the region enclosed by the bounding function. It is only required to calculate the angle, and compare the two distances obtained: the distance of the point under consideration, and the radius obtained from R(θ).

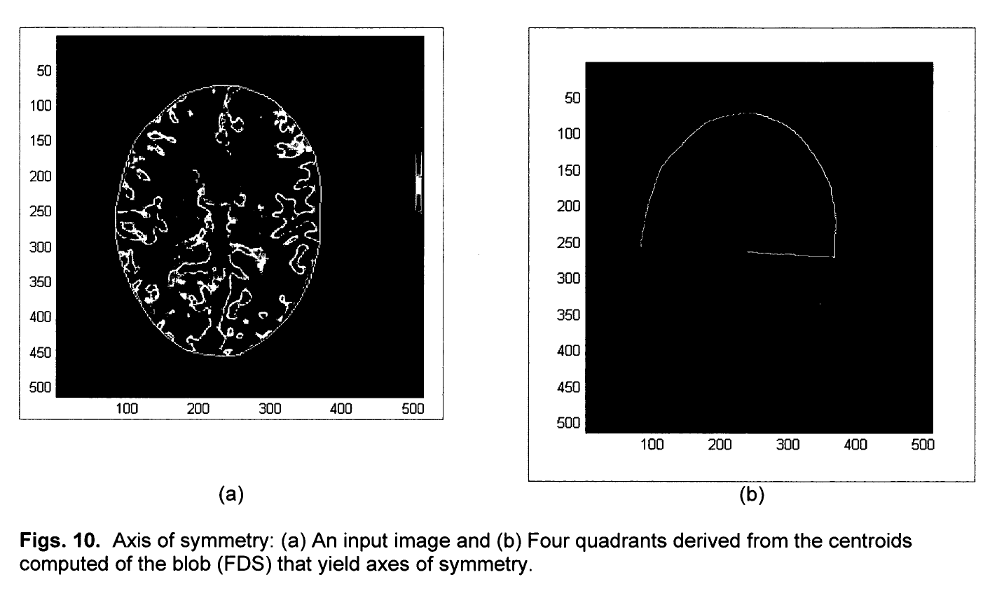

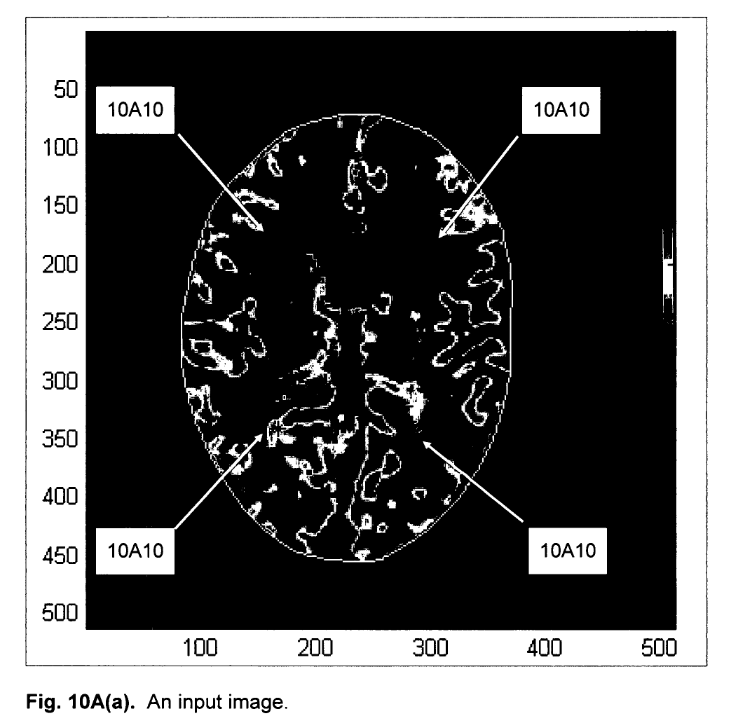

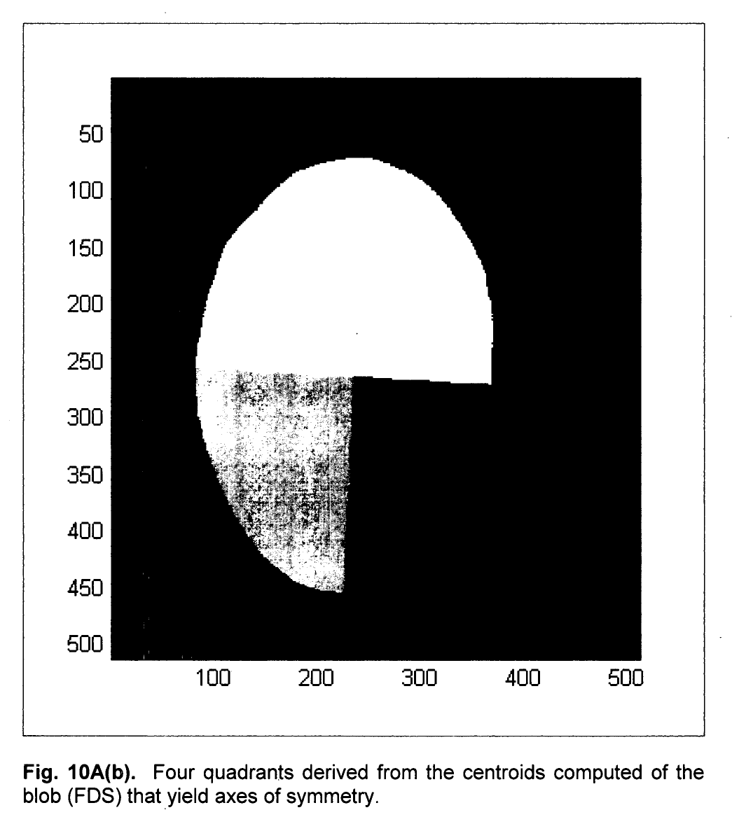

Having this periodic bounding function allows calculation of the Fourier Shape Descriptors (FSD) of the bounding function (region), calculation of the centroids of any angular section of the object, and derivation of axes of symmetry, as shown, for example, in FIG. 10.

Image Unwrapping

The FDS of the shape allows for unwrapping the corresponding boundaries of the original image and the “blob” over the angle θ, and this new representation allows for multiple manipulations of the original image. For example, image unwrapping allows for comparing the distance between two boundaries.

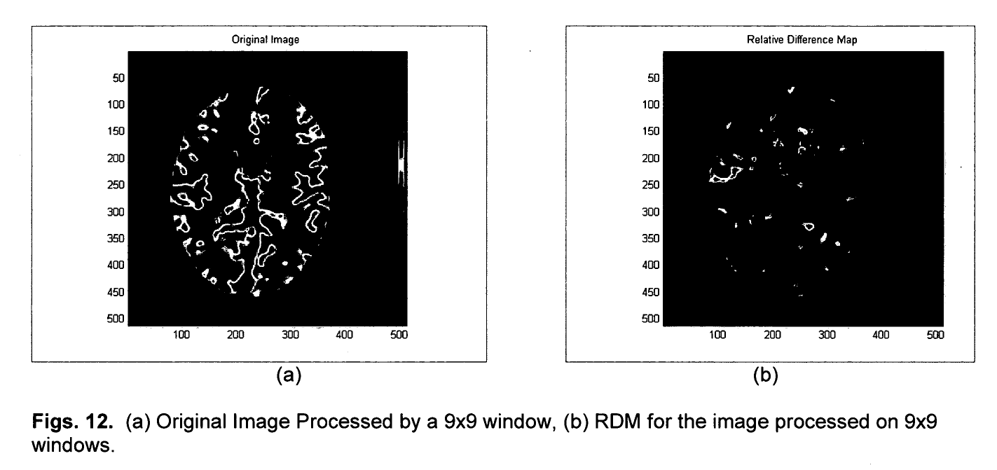

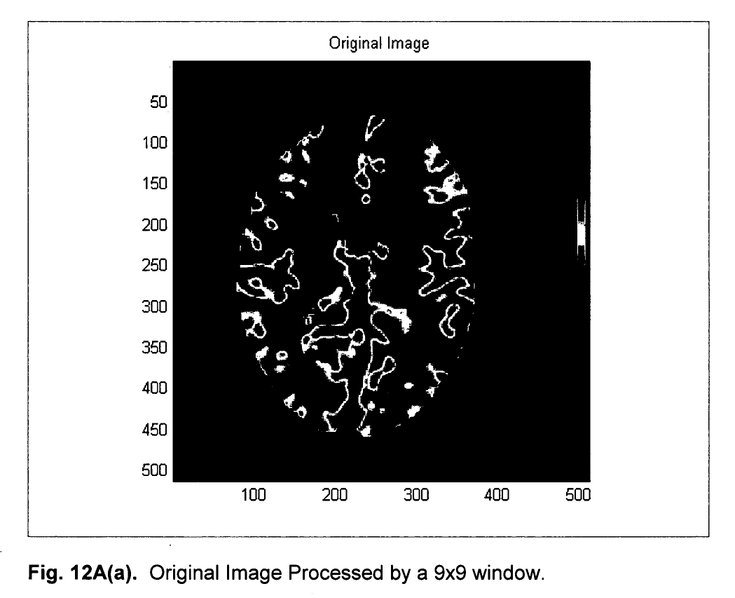

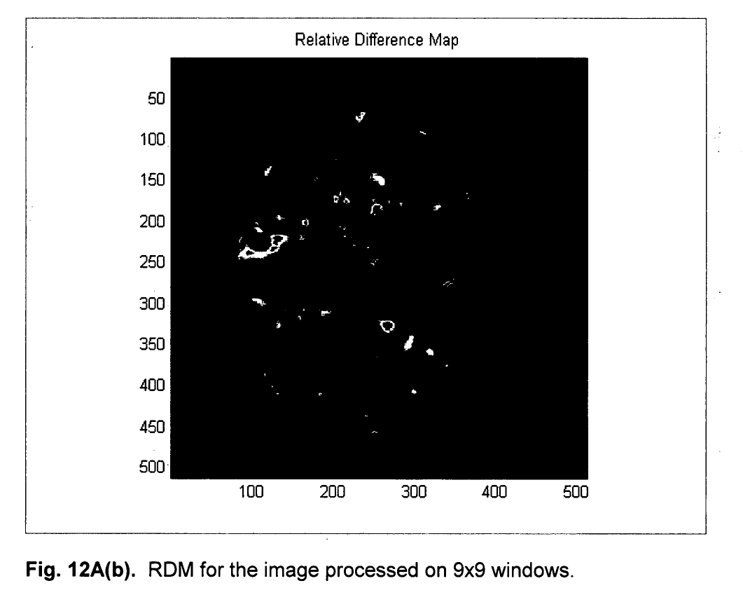

Following unwrapping, each 2D image can be viewed in a dual representation: original and unwrapped. The unwrapped image can be used for: (a) selection and calibration of axis of symmetry, FIG. 11(a); (b) the comparison of analogous portions of the brain to compute a relative difference map, FIGS. 12; (c) interactive removal and selection of regions from the center of the image; and (d) selection of generic angles that are used to approximate the 6 cerebrovascular territories in the brain, as shown in FIG. 11(b).

Moreover, after the data is unwrapped, each of the radii can be renormalized to equal length, to facilitate the comparison of features in the left and right halves of the image. The stretched image can thus provide a registration of the left and right data that allows the direct comparison of relevant points in the original image. In addition, a window may be applied prior to the “unwrapping” and “stretching” to compensate for anatomical differences in the dataset, and errors in the selection of the axis of symmetry. After the completion of relevant operations performed on the “stretched” image, the results can be re-sampled back into the same shape as the “original” dataset. When multiple pixels would map to the same point, the results may be averaged, or the maximum taken to create a composite element.

Computation of Plane of Axis of Symmetry in a CTP Images

To exploit symmetry, the image can be, for example, automatically aligned along an axis of symmetry and the left-to-right test that highlights discrepancy in the dataset can be implemented. Two types of axes of symmetry can be automatically generated for:
(a) Symmetry of content. The axis of symmetry that passes through a focal point (e.g., centroid of anatomical region of interest). Once a convex hull and corresponding Fourier Shape

Descriptor have been computed for a given image, pixel intensities can be used to compute the centroids in the image, and derive the axis of symmetry;
(b) Symmetry of shape. The axis of symmetry passing through the centroid of the unwrapped data set. The perfusion-weighted image is unwrapped onto a rectangular stretcher with the axis of symmetry as a vertical line in the middle. The symmetry axis is adjusted by finding the indentation point in the unwrapped stretched image.

While the symmetry of content may also be useful, symmetry of shape was used to compute an axis of symmetry in a CBF image as a pre-processing step for computation of the RDM.

Computation of Relative Difference Maps (RDMs)

RDMs were calculated according to this automated method, using principles similar to those utilized by the earlier manually aided method with the difference that the axis of symmetry was computed automatically. Given an input image and its unwrapped representation with the axis of symmetry, the RDM was computed, as shown in FIG. 12. From the unwrapped representation of the RDM, the left and right hemispheres were derived.

Automated Registration of Cerebrovascular Territories.

Because the most relevant application of CBF quantification is the determination of regional CBFs in the six major vascular territories of the brain (i.e., the anterior cerebral artery [ACA], middle cerebral artery [MCA], and posterior cerebral artery [PCA] territories in (each of the left and right hemispheres), an automated method for dividing brain images into these territories so that each region could be analyzed independently was sought. Achieving this task was difficult for two principal reasons. First, the territories are irregularly shaped. Second, the territories differ greatly in both shape and size. Since the results of the quantified method were presented as histograms of the RDM values in each of the vascular territories, and the geometric and statistical features of the histograms were to be used for clinical interpretation of patients with SAH, it was believed that defining generic, simplified estimated vascular territories was a good first approach to automation of the process.

Thus, initially, the vascular territories were delineated using anatomical landmarks that were operator-dependent and time consuming. Because of these inherent limitations to the manual method, a method for automatically dividing the brain tissue into generic vascular territories in a reproducible, and reliable way was developed. The advantages of such an automated tool in the clinical setting are obvious, as this is the way the brain is conceptually divided by clinicians treating patients with suspected or known brain ischemia.

FIG. 11 outlines the steps of this method. Briefly, this method uses unwrapped input CBF image, to roughly divide the brain into generic territories using estimated angles that roughly estimate the ACA, MCA, and PCA territories. These sub-divisions are then mapped into a corresponding RDM image. This ws achieved, as shown in FIG. 11(a), by walking from θ=0 to θ=27r, and from radius 0 to the length of two radii, defining vertical bars corresponding to the lines that partition the unwrapped image into six vascular territories. The angles that were found to best estimate the territories were: (a) right posterior cerebral artery at

$$θ = ( 0 , \frac{π}{6} ) ,$$

(b) right middle cerebral artery at

$$θ = ( \frac{π}{6} , \frac{5}{6} π ) ,$$

(c) right anterior cerebral artery at

$$θ = ( \frac{5}{6} π , π ) ,$$

(d) left anterior cerebral artery at

$$θ = ( π , \frac{7}{6} π ) ,$$

(e) left middle cerebral artery at

$$θ = ( \frac{7}{6} π , - \frac{π}{6} ) ,$$

(f) right posterior cerebral artery at

$$θ = ( - \frac{π}{6} , 0 ) .$$

When mapped into the original image, (FIG. 11(b)), the wedge-shaped vascular territories are defined by these generic angles, and plotting the relative difference values for all the pixels within each territory generated the RDM histogram.
Preliminary Results: RDMs with Manual Demarcation of the Axis of Symmetry and the Vascular Territories

Preliminary results suggested the potential promise of the RDM method for characterizing asymmetry in CTP images. The three illustrative cases described below were selected because they represent the spectrum of potential CBF alterations. The images were presented as RDMs processed from CBF maps.

Patient 1: The patient was a 43 year-old man who presented with Hunt and Hess grade II SAH. Cerebral angiography disclosed a large (2 cm) MCA aneurysm, which involved the lenticulostriates. The patient's clinical course was unremarkable from a neurological standpoint, with no episodes of CVS detected by daily Transcranial Doppler sonography (TCD), cerebral angiography, or routine neurological examination. On SAH day 5, the patient underwent CTP, depicted in FIG. 13. Regional histograms of vascular territories demonstrate relatively minimal deviation of the curve from zero in all territories, indicating relatively normal levels of perfusion throughout the brain.

Patient 2: The patient was a 77 year-old woman who presented with symptoms consistent with left MCA infarction. FIG. 14 clearly demonstrates large wedge shaped region of severe hypoperfusion in the MCA territory consistent with acute proximal occlusion of this vessel. When this image was processed using the exemplary algorithm, a clear peak on the far right was seen in the histogram representing the left MCA territory that is consistent with the theoretical stroke curve shown in FIG. 2. The histograms for other vascular territories have significantly smaller means, and thus many appear “normal.”

Patient 3: Patient ws a 36 year-old woman who presented with Hunt and Hess grade IV SAH. Cerebral angiography disclosed a 4 mm×3 mm right anterior choroidal artery aneurysm. Her neurological examination improved significantly postoperatively. However, on SAH day 5, she experienced an acute decline in mental status, however neurological exam demonstrated no focal neurological deficit. A CTP performed at that time, shown in FIG. 15, was read as normal by an attending neuroradiologist. The patient subsequently developed bilateral arm weakness and was taken for angiography, which revealed a severe vasospasm of the right and left MCA and right ACA. This spasm was treated with angioplasty, with significant clinical improvement. However, follow-up MRI two months later demonstrated an old cerebral infarction in the right frontal lobe. The RDMs demonstrate significant regions of side-to-side asymmetry in the Left MCA and Right ACA territories, consistent with the results seen at angiography. The histograms of these regions, while not as striking as those seen for Patient 2, nevertheless demonstrate significant increases in frequency of significantly mismatched pixels (i.e., shift of curve to the right).

Thus, RDMs and their histograms derived for vascular territories were successfully generated using the automated method described above and the results closely approximated those obtained by careful manual delineation. In the histograms shown, a bucket size (an interval in the x axis of the histogram) of 0.02 was used and the number of pixels in each bucket was depicted using normalized scale consistent across all histograms.

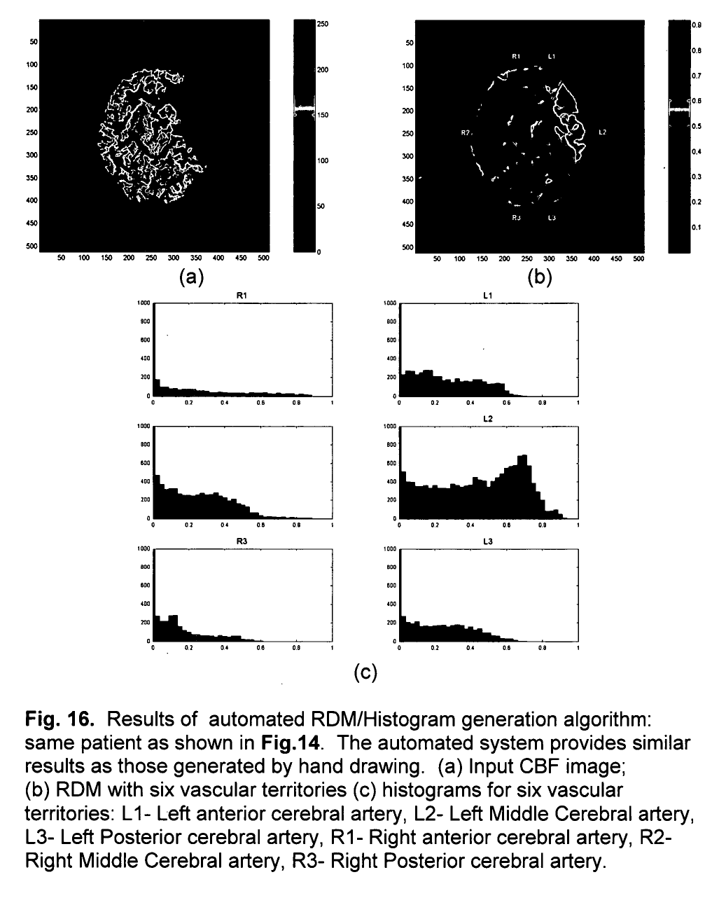

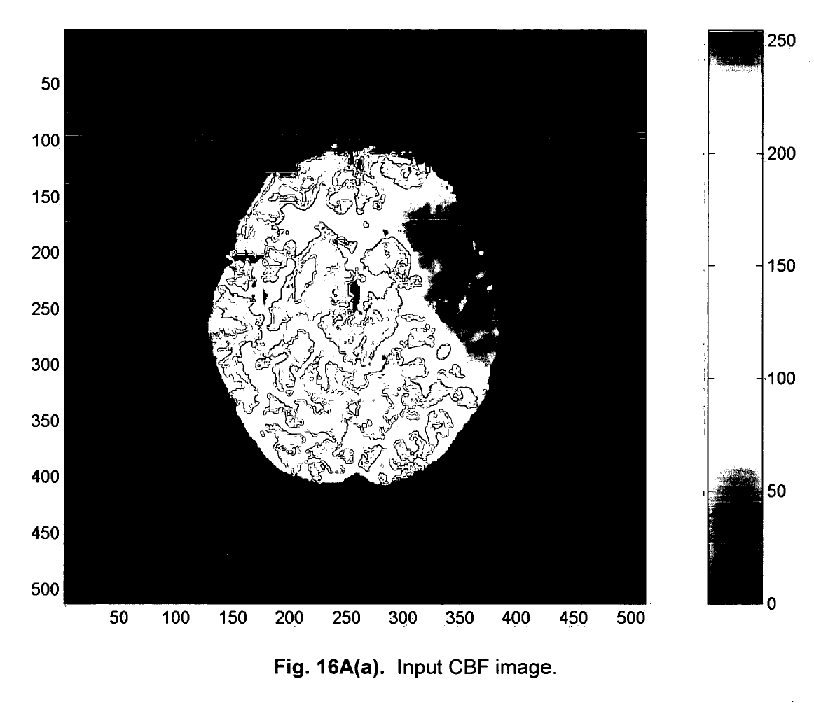

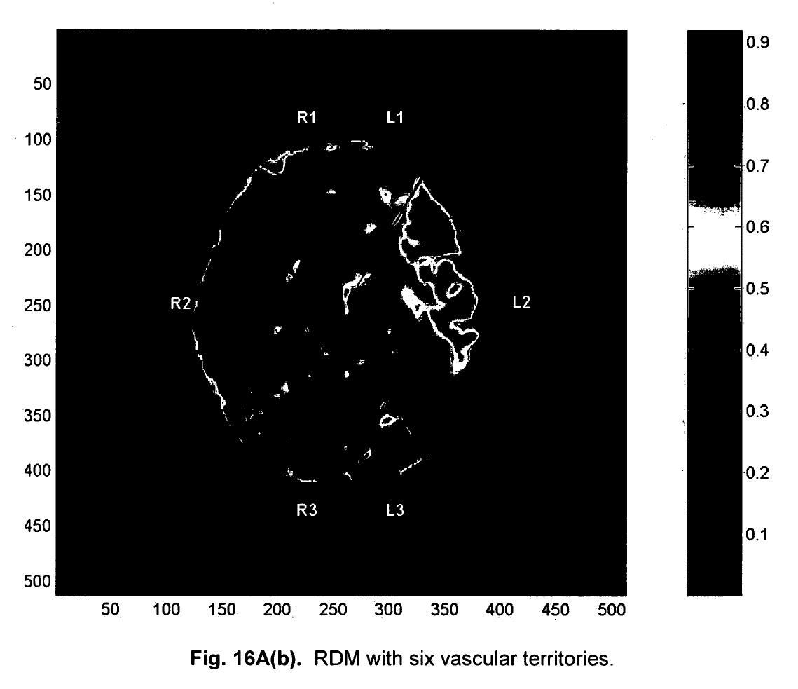

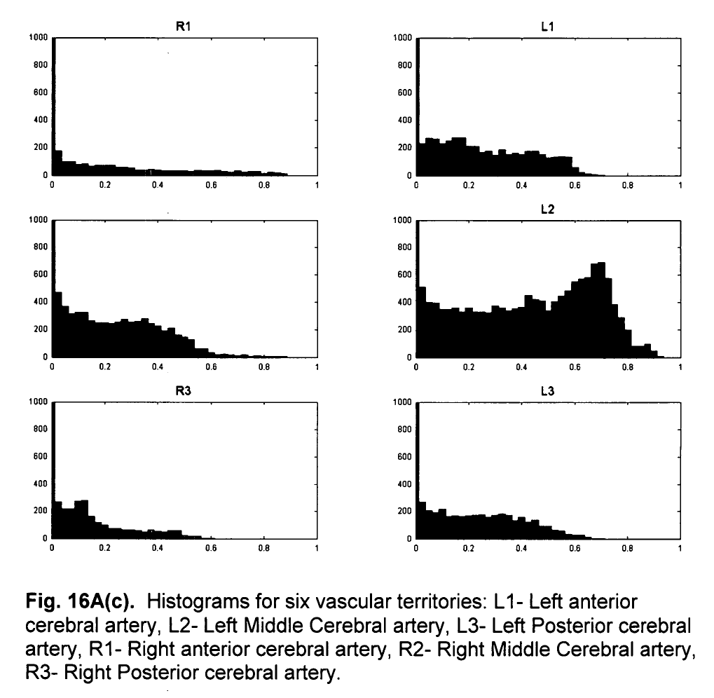

In all cases, the automated process produced similar histograms to that obtained by the manual method. For the sake of brevity, the results for one patient, Patient 2, are shown in FIG. 16, who at the time of the scan, was suffering a large left MCA ischemic stroke. These data closely mimic the results obtained by hand drawing territories. This demonstrates that in exemplary embodiments of the present invention, this process can be adequately automated to rapidly generate RDM/histogram data of equal quality to that done by a clinician.

Interestingly, the creation of the exemplary algorithm necessitated the development of a number of novel methodologies for addressing specific issues of image processing and analysis that have the potential for use in other medical imaging application. A fundamental pattern of patho-anatomical alteration common to numerous disease processes is the development of side-to-side asymmetry within the organ structure. Consequently, looking for asymmetry is a common method used throughout radiology. While gross, homogenous side-to-side asymmetry probably does not require numerical analysis of image characteristics using a computer, subtler, patchy asymmetry may be missed routinely.

The disclosed algorithm thus provides a robust method for detecting the edges of an organ, determining its axis of symmetry, and characterizing the degree of side-to-side mismatch using regional histograms. While application of these methods to other regions of the body might be limited by a lack of true side-to-side symmetry, the potential applications of a fully developed, automated method for complex characterization and quantification of asymmetry are numerous and span, for example, multiple fields of science and medicine.

IV. Enhanced Techniques for Asymmetry Quantification in Brain Imagery

Medical image segmentation, that is, a process of identifying and delineating anatomical structures and other objects in images, still largely remains an open problem, in spite of several decades of research from various imaging modalities. There are many brain segmentation approaches evolved from low level image operation such as thresholding, edge detection, mathematical morphology, to more sophisticated image processing methods such as statistical classification active contours, neural networks, fuzzy connectedness, and hybrid segmentation methods. However, clinical image analysis indicates that to successfully differentiate between organ and tumor tissue image information alone is insufficient. For example, if a tumor shows insufficient contrast against the healthy brain tissue, the active contour classification requires a selection of seed to initialize segmentation, hence the method is not fully to automated. Other statistical classification methods are also limited due to overlapping intensity distributions of healthy tissue, tumor, and surrounding edema. In the digital anatomical atlas based segmentation prior knowledge about normal brain anatomy is used, including the size, shape and location of anatomical structures. However, the shape and other characteristics of tumors and other brain pathologies are highly variable, thus representing prior knowledge is not always possible. In fact, an adaptive template moderated classification (ATMC) method that combines the statistical classification with anatomical knowledge has been proposed. The algorithm involves iterative process of classification of patient's data and nonlinear registration to match the anatomical templates of a digital atlas with the brain anatomy of the patient. However, such atlases, obtained from manually segmented MR imaging are not always available and such an approach has a limited use.

Thus, in what follows, a method of identifying and delineation of brain pathology by analyzing the opposing sides of the brain is described. In another words, the need for a digital atlas is replaced with the cross-registration between opposing hemispheres of the brain. Because brain exhibits high level of bilateral symmetry and this symmetry is distorted in a presence of a pathology, we utilize the inherent left-right symmetry in the brain was utilized and the healthy side of the brain used as a prior template to statistically enhance the differences in high dimensional feature space. With a fully automated system brain abnormalities such as tumors, stroke, and other pathologies were segmented. Spatial prior of a generic statistical normal human brain atlas was replaced with the subject-specific left-to-right symmetry information derived from the subject's brain images. Human and rat data were used with stroke and tumors to demonstrate the capabilities of the disclosed method. A pipeline was defined that can accurately capture the asymmetries between two hemispheres, quantify the discrepancies and segment the pathologies. Additonally, examples that illustrate the pipeline are provided.

The capability of the method illustrated with examples ranging from tumors in patient MR data to animal stroke data. The method can be used to improve segmentation of pathology in structural brain images and as quantification of perfusion-weighted tomography. There are a number of clinical applications in neuro-radiology where this method can be useful: Identification and segmentation of ischemic stroke in animal, and human models, quantification of magnetic resonance perfusion (MRP) changes in patients with cognitive deficits following carotid endarterectomy, and objective quantification of perfusion-weighted computer tomography in the setting of acute aneurysmal subarachnoid hemorrhage, to name a few examples.

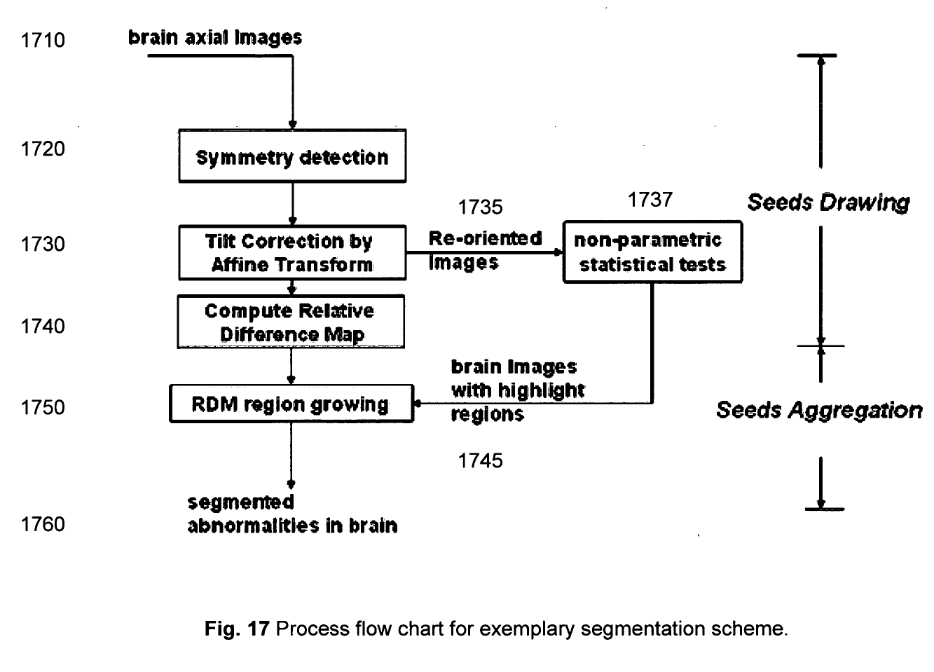

An automated segmentation tool, depicted in the flow chart of FIG. 17, was developed that utilizes the inherent left-right symmetry in the brain. In a first step 1720 the symmetry axis is computed and at 1730 the tilt of the head is corrected by applying standard alone transform. The detection of slight variations in the left to right brain imagery is complicated by normal differences in the anatomical structures occurring in the data set. Therefore, at 1737 we utilize the apparent bi-lateral symmetry in brain imagery through the application of non-parametric statistical tests operating on the pairs of samples and non-parametric statistical tests on local averages. This technique can highlight regions that can be further examined in a “statistical” sense. The highlighted region 1745 can be used as “seeds” for later propagation in the difference map that quantifies differences between brain hemispheres.

Such statistical tests provide the “likelihood” of the difference to appear randomly given the samples, that has 1: N chance of occurrence without un underlying difference being present. Finally at 1750 a region is grown within the difference map yielding a segmentation of an abnormal target in the brain 1760.

As shown in FIG. 17, the major components of the disclosed segmentation pipeline consist of two consecutive steps, seeds drawing and seeds aggregation. Unlike most current region based segmentation approaches, seeds placement is fully automated. Seeds are understood as the occurrence of the regions with statistically minimal chances of an underlying symmetry being present.

In radiological brain images, to measure differences in bi-fold mirror symmetry the axis of symmetry must be computed and the image “self-registered” to this axis. After detection and registration of the image to the axis of symmetry, the data can be checked for significant differences in the image in regions collocated relative to the given axis of symmetry. This approach defines the essence of the relative difference map (RDM) method that quantifies and highlights asymmetric areas in two brain hemispheres. In this study, we refined the method of selecting an axis of symmetry and computation of relative asymmetry with respect to the axis of symmetry. Our method allowed computation of the RDM for an image. A pair of symmetric windows (image elements) were selected, being ordered sets of points, about the axis of symmetry and a set of relative differences were computed with the associated statistics while scanning simultaneously both hemispheres. Thus, the seeds placement was further composed of the following sub-components: symmetry detection (1720), tilt correction (1730) and asymmetry measurement by non-parametric statistical tests (1737).

Symmetry Detection and Asymmetry Measurement (Seeds Drawing)

Many clinical images are misaligned, tilted, a phenomenon that may be caused, for example, by the immobility of the patient, inexperience of the technician, or the imaging device itself, symmetry detection and correction of the misalignment comprises the first entry of the pipeline. Various approaches to detecting, analyzing, measuring and applying symmetry in image analysis have been suggested.

We summarize the seeds drawing algorithm into the following steps:
1. Identifying the axis of symmetry;
2. Affine transform to re-center and re-orient the image;
3. Conducting statistical symmetry measurement to highlight the “sufficient” asymmetry.
   Identification of Symmetry Axis

Let us assume that we have a single object in of interest. The area of the region R=(xy)εR:I(xy)>α,α>0, with the given the characteristic function can be found as follows:
A=∫∫_{I}χ(x,y)dxdy=∫∫_{R}dxdy (1)

With the integration over the whole image I, and A defined as the 0th moment of R, then the center of the object can be computed, that is that point where all the mass of the object could be concentrated without changing the first moment about any x-axis.
x̅=∫∫_{R}xdxdy (2)
y̅=∫∫_{R}ydxdy (3)
where ( x̅, y̅) is the position of the center of area. The technique follows rays r(θ) emanating from the centroid ( x̅, y̅) to the boundary elements of x_{R}. The intersections of the ray with the boundary of the region are recorded. Obviously, when the centroid is interior to the region of interest (ROI) all of the points on the boundary are path connected to centroid, and a ray extending from the centroid (a straight path) will intersect at least one point on the boundary. 12 The rays are parameterized by the discrete approximation to θ=kΔθ, kεZ. (θ that is discrete is used to simplify notation). The value of Δθ is chosen to insure that the step doesn't exceed \sqrt{2} at a given maximum radius. For example, by selecting the points furthest away from the centroid this constructs the star shaped object, with each of the rays indexed by the angle. Any image I(x, y) [symbol] (r,θ) where the origin (0, 0) is in the centroid of the image, with the support of the function being χ_{R}. For an object with a well-defined boundary, there are lines L_{K} extending from the centroid ( x̅, y̅) of length and direction (r(θ),θ). In the polar representation, to determine the k-fold mirror symmetric axes we seek the K values φ1, φ2, . . . , φk ar sought that minimize the distance (in general)

$${d}_{φ}^{P} ( Right , Left ) = \sqrt[p]{{\int }_{θ = 0}^{π} {\vert r ( ϕ + θ ) - ( ϕ - θ ) \vert}^{P ⅆ θ}} \qquad ( 4 )$$

Specifically, for p=1,

$${d}_{φ}^{1} ( Right , Left ) = {\int }_{θ = 0}^{π} \vert r ( ϕ + θ - r ( ϕ - θ ) \vert ⅆ θ \qquad ( 5 )$$

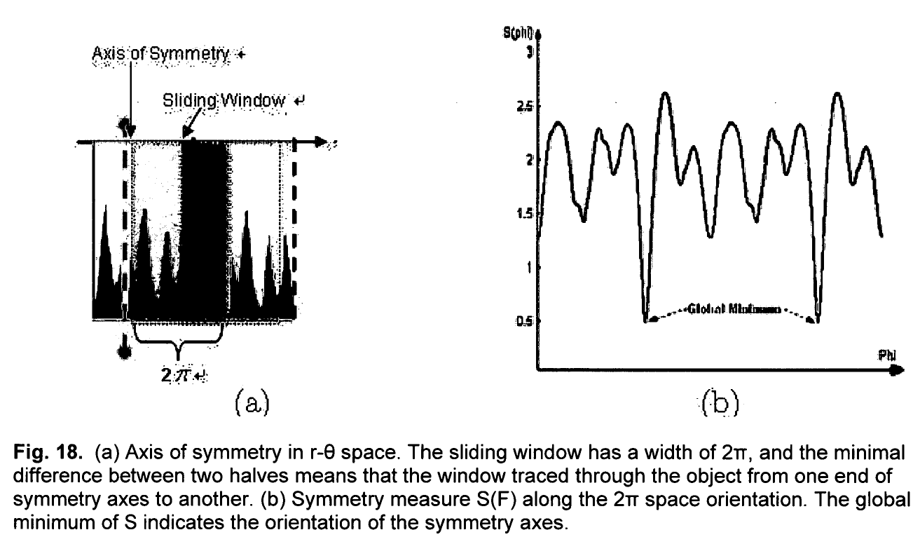

with the best symmetry axis being
φᵣ=arg min d₁₀₀ ¹(right, left) (6)
the resulting φᵣ that minimizes the distance are the minimum torque arms. Therefore, the problem of identifying symmetric axes can simplified to spot the global minimum of symmetry measure S in r−θ space. This can be implemented, for example, as comparing the left half and right half of a sliding window across the entire r−θ space, as shown in FIG. 18(b). The window width is equivalent to the total amount of rays completing the transverse of polar space 2π and the window height is equivalent to the maximum radius preset in an exemplary application. Therefore, what is essentially sought is such a state of equilibrium where the sum of all the difference between the left pixel and right counterpart over the space π, is the minimum (ideally zero), as shown in FIG. 18.
Affine Transform to Re-Center and Re-Orient the Image

Supposing that an image, f, defined over a (w, z) coordinate system, undergoes ageometric transformation to produce an image, g, defined over an (x, y) coordinate system. This transformation can be expressed as [xyl]=[wzl]T, where T is called transformation matrix.

$$T = [ \begin{matrix}cos ( θ ) & sin ( θ ) & 0 \\ - sin ( θ ) & cos ( θ ) & 0 \\ {c}_{x} & {c}_{y} & 0\end{matrix} ] \qquad ( 7 )$$

θ is the rotation angle, and is the translation offset between centroid cₓ, c_{y} of the brain and the center of the image.
Application of Statistical Tests to Highlight Asymmetry

The described method allows for the computation of an RDM for an image. A pair of symmetric windows (image elements) can be selected, being ordered sets of points, about the axis of symmetry and a set of relative differences with the associated statistics can be computed while scanning simultaneously both hemispheres. To create pairs of image elements the right or left set of pixels can flipped left to right (e.g., X̅_{R}) allowing one to group pixels into regions that should be significantly similar. The neighborhood of a pixel can be defined as a set of offsets from a row of columns, as in Equation 8. The neighborhood can be used to create the set of image values about
N=(Δr₀, Δc₀), (Δr₁, Δc₁), . . . , (Δrₖ, Δcₖ) (8)
S_{N}: X(r, c)=X(r′,c′):(r+Δr, c+Δc), (Δr,Δc)εN (9)
the point (r, c). The boundary and background points should be identified and treated appropriately. Secondly we use the Wilcoxon rank sum test can be used for equal median on the paired elements and the p value can recorded as in Equation 10.
p(r, c)=ranksum(S_{N}:X_{L}(r, c), S_{N}:X_{R}(r, c)) (10)
The Wilcoxon rank sum test was selected since it compares the median of the pair-wise differences of the two data sets without the restrictions present in the Student's t-test. Also, other statistical tests can be substituted. The test p represents the probability of observing the statistic by chance if the medians are equal. The calculated probabilities are used to estimate regions in the image that exhibit significant difference by applying a threshold as in Equation 11 to obtain a characteristic function.
χₐ(r,c)={1,∀(r,c):p(r,c)<α,α>0, otherwise 0} (11)

Due to the natural variation present in the biomedical imagery, it is possible that there is a neighborhood that minimizes the p of the rank-sum test is slightly offset from the point of bi-fold symmetry. To compensate for this, the technique can consider a set of offsets, as in Equation 12, that can be used to shift the one of the neighborhoods to compensate for asymmetries and accumulate the p values for all offsets to obtain an average p value as in Equation 13.

$$\begin{matrix}O = ( Δ {r}_{0}^{′} , Δ {c}_{0}^{′} ) , ( Δ {r}_{1}^{′} , Δ {c}_{1}^{′} ) … ( Δ {r}_{k}^{′} , Δ {c}_{k}^{′} ) & ( 12 ) \\ \begin{matrix}\overline{p} ( r , c ) = \frac{1}{\vert O \vert} {\sum }_{( Δ r , Δ c ) ∈ O} ranksum ( {S}_{N} : {X}_{L} ( r , c ) , \\ {S}_{N} : {X}_{R} ( r + Δ r , c + Δ c ) )\end{matrix} & ( 13 )\end{matrix}$$

RDM Segmentation: Region Growing Within the Difference Map (Seeds Aggregation)

The basic idea behind region growing is aggregating pixels (voxels) that are adjacent and belong to the same tissue type with fairly homogeneous grayscale properties. Region growing approach consists of two major steps: first, selection of a set of seed points; second, growing of the region by appending the neighboring pixels (voxels) that have satisfied similarity criteria. In our segmentation pipeline, the seeds have been drawn from the preceding step where the “seeds” can be defined as the pixels where the most statistically significant difference appears. Hence we provide a full automation of seed selection.

The stopping criteria of region growing is formulated based on intensity values of difference map. Since the “seed” assumes a certain number of points, we can calculate its mean and variance. Therefore, the value of each candidate pixel is compared with the average intensity of the seed region. A threshold value is used to test if the candidate is sufficiently similar to the seeds. In addition, if the pixel in question is 8-way connected to one or more seed values, then the pixel is considered a member of one or more regions. The region is being grown within difference map and the threshold value is characterized by the standard deviation of the seed region.

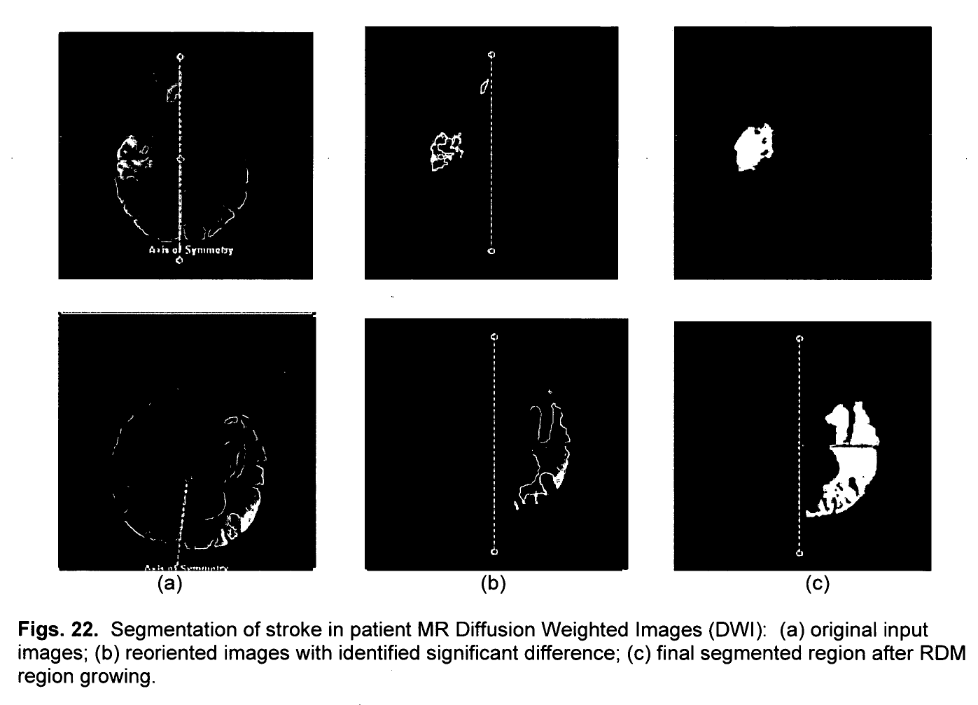

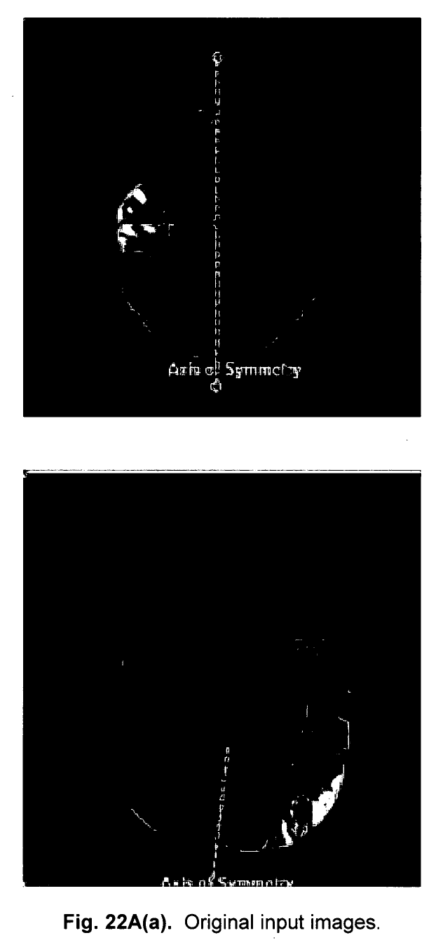

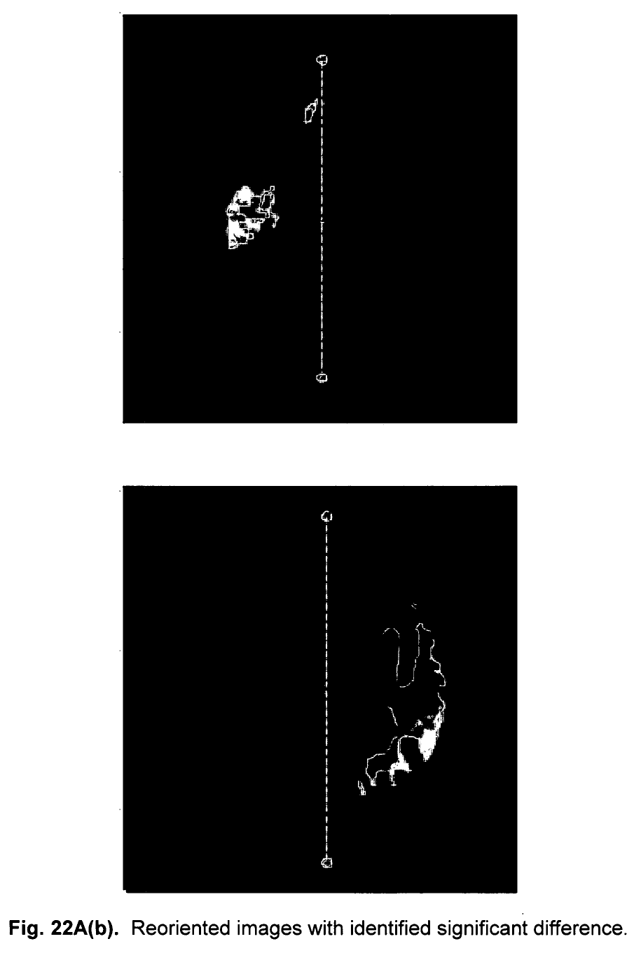

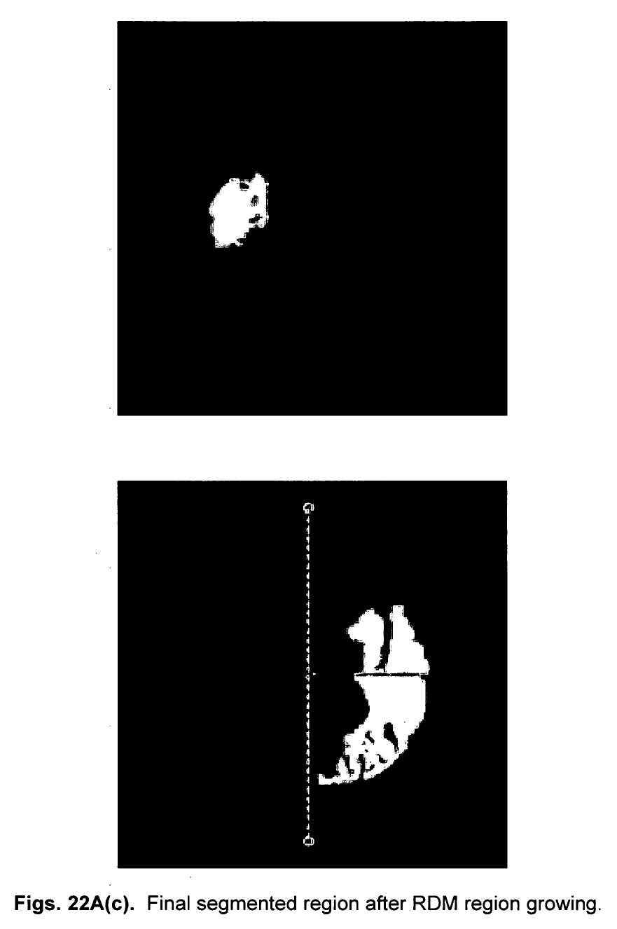

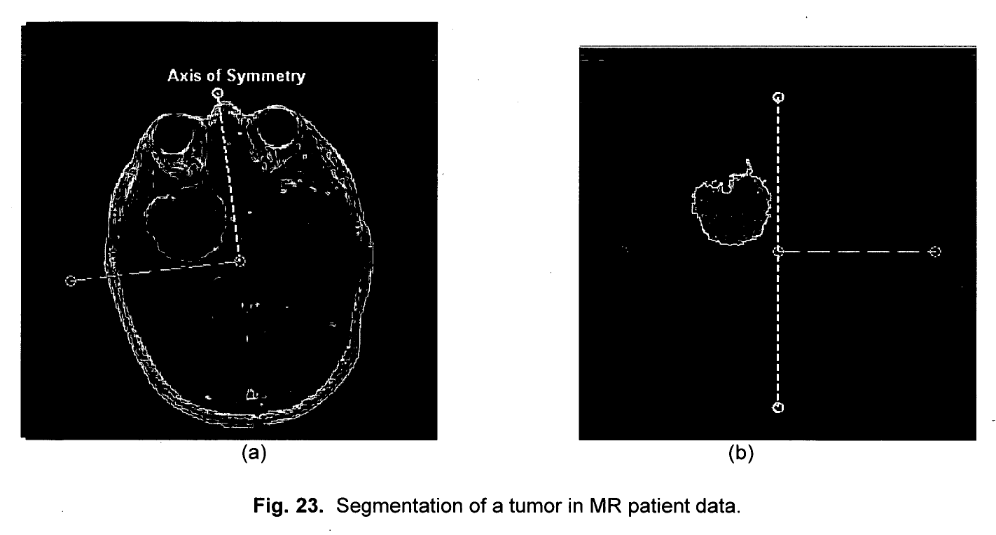

The output of region that can be grown with the disclosed methods is a labeled region (volume), where the label indicates the membership of a pixel (voxel) in a segmented object. A label of zero indicates that the voxel was not assigned to any object. In cases where brain has multiple lesions, the segmented image has multiple distinctive non-zero labels. For example, in FIG. 22, the brain has two stroke areas; therefore, it is being marked with two labels. Therefore, the proposed segmentation pipeline does not require a user to select an initial seed and the segmentation is fully automated.

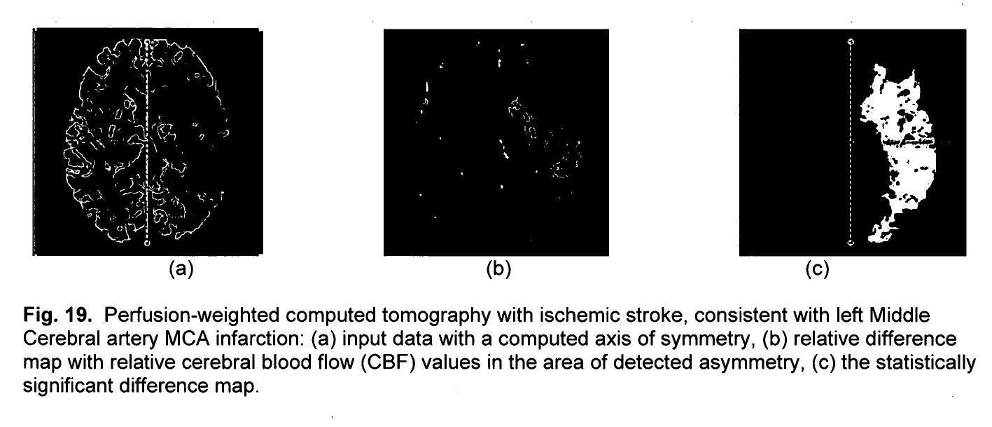

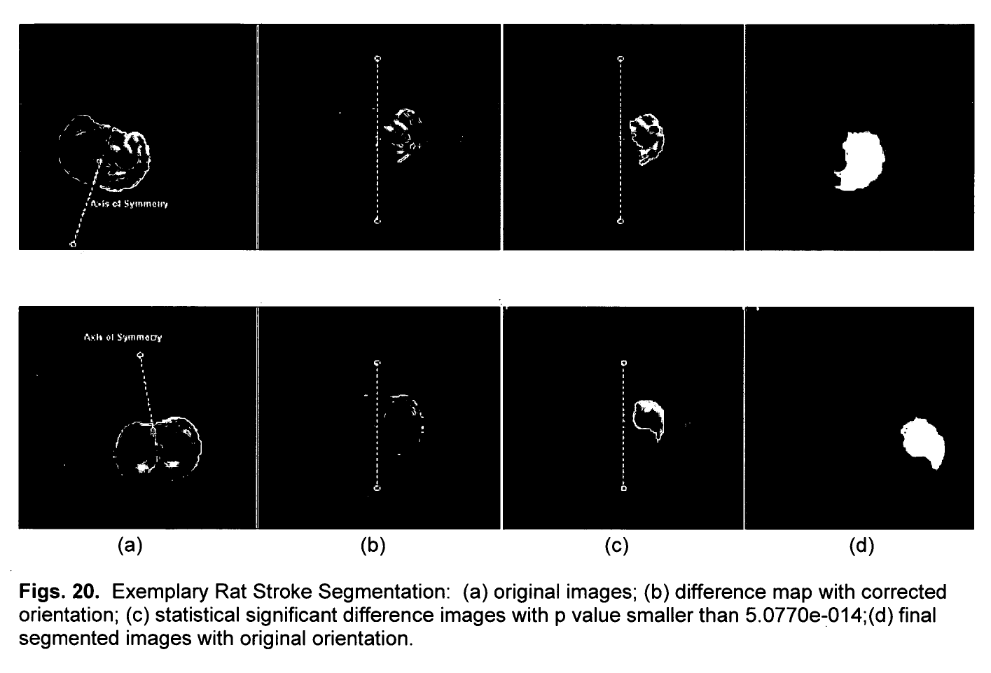

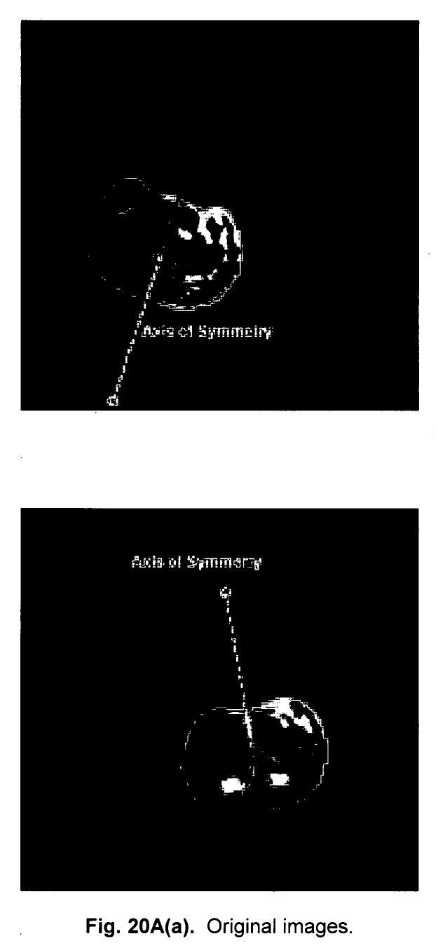

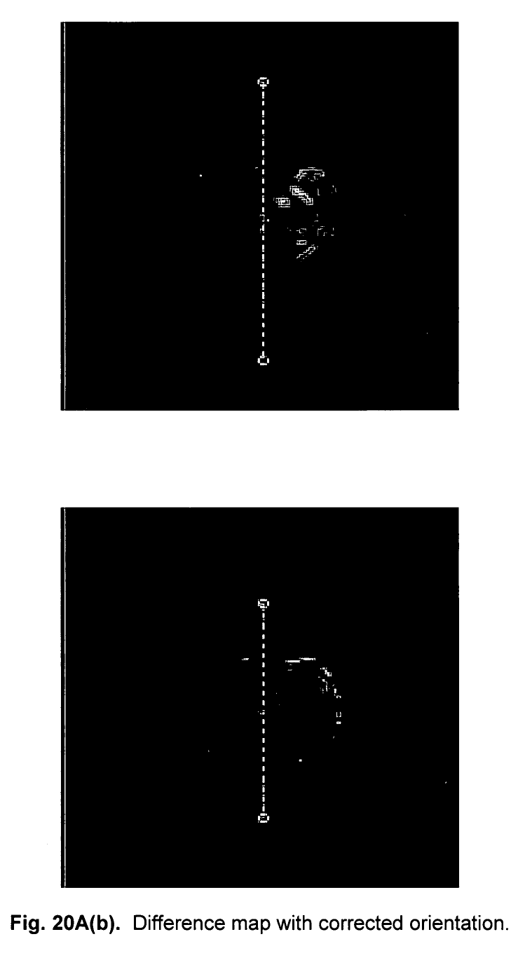

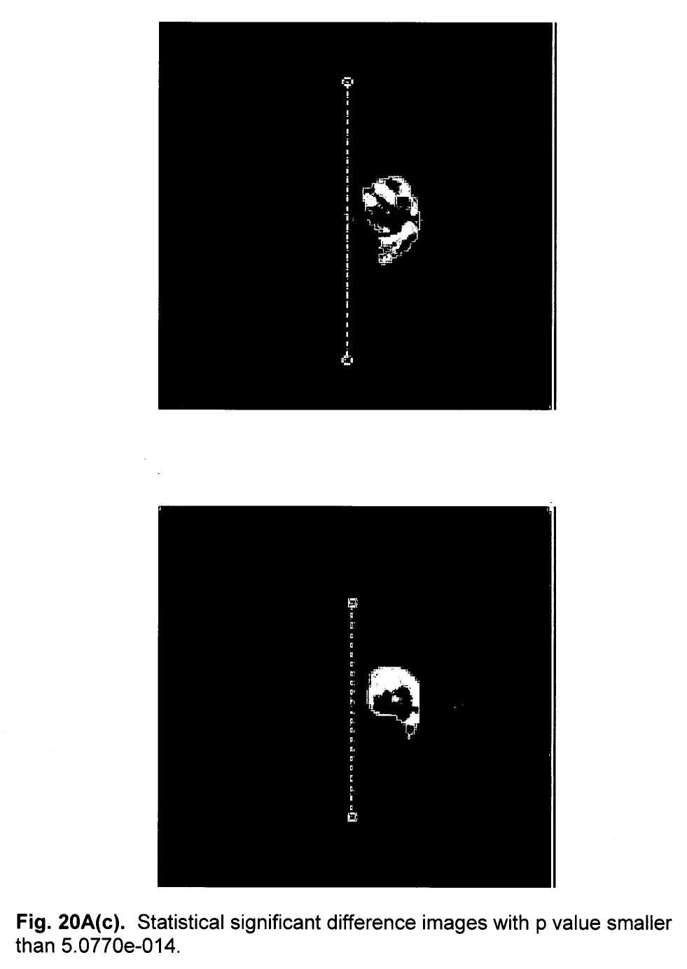

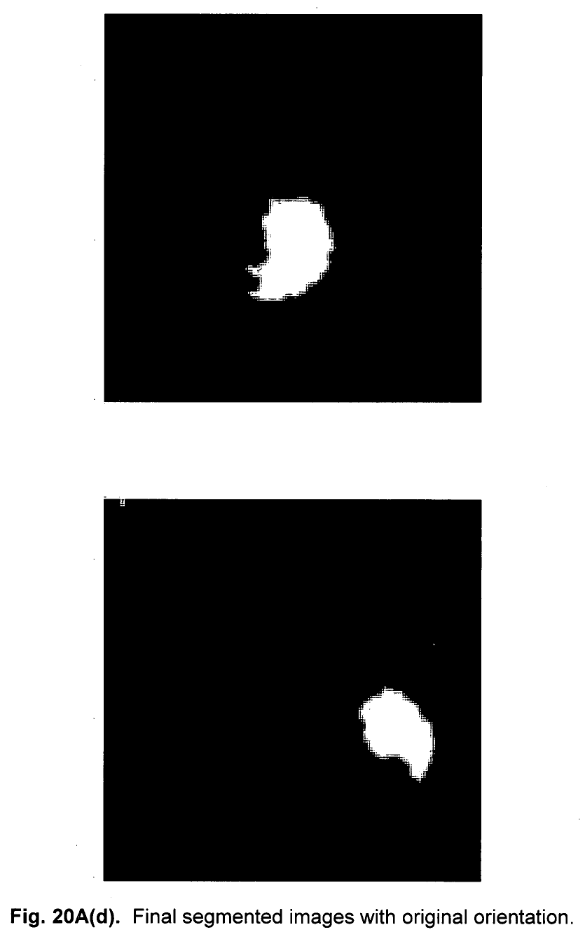

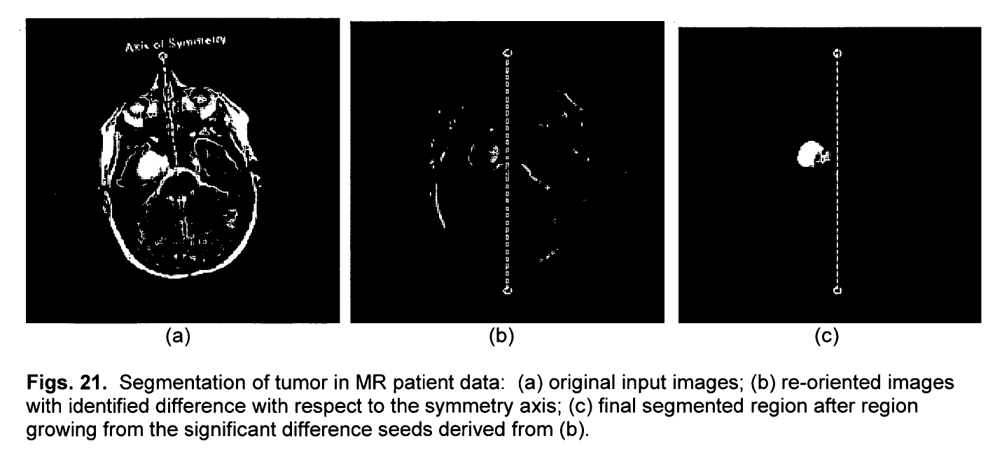

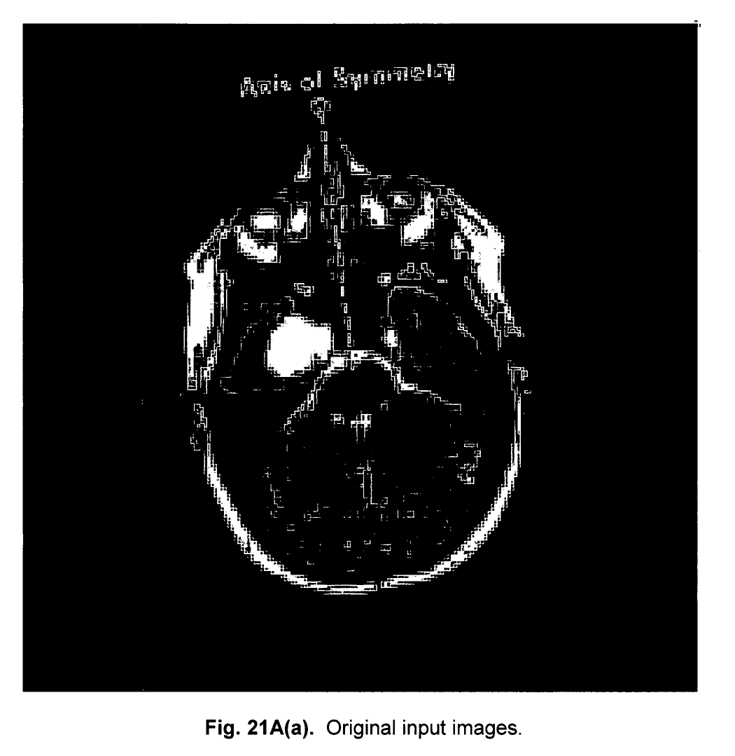

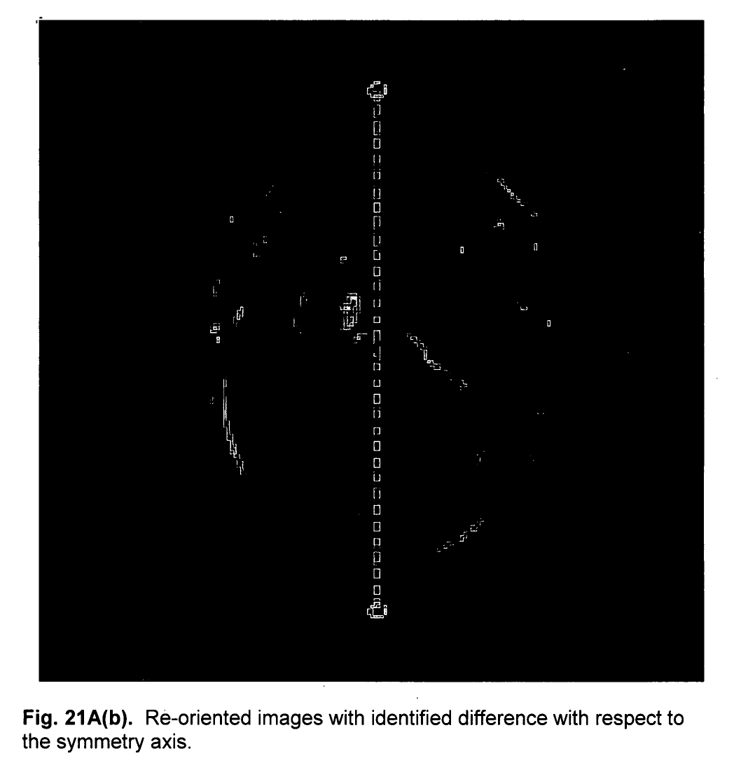

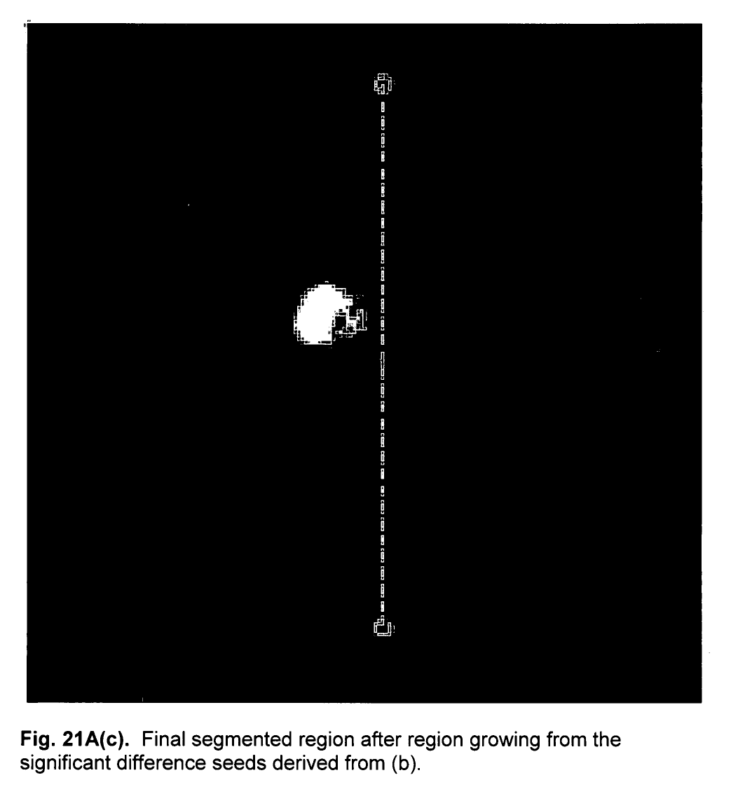

Illustrated below is the technique on a number of clinical and animal examples. Computation of relative asymmetric regions is used for quantification of perfusion-weighted images, and pre-segmentation of brain tumors from structural magnetic resonance imaging. The relative or original values are shown superimposed on the detected area of asymmetry. In quantification of perfusion-weighted images the relative values of perfusion parameter are displayed and highlighted in the detected area of asymmetry, FIG. 19. In pre-segmentation of brain tumor from structural MR data, FIG. 21, the original values are shown on the detected area of asymmetry.

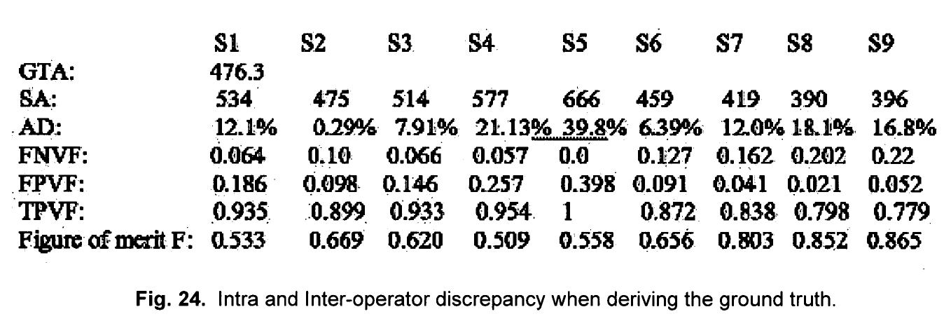

To measure accuracy of a segmentation method against a “true” delineation, a surrogate of ground truth, three parameters can be computed, for example: True Positive Volume Fraction (TPVF), False Positive Volume Fraction (FPVF), and False Negative Volume Fraction (FNVF). The automatic segmentation method on rodent models of focal cerebral ischemia that have been developed to investigate stroke therapy. In the study, 8 adult Wistar rats were used. Surrogate of ground truth of the stroke regions was obtained, for each rat, by repeated manual delineations by experts, where each expert repeated it three times, following a strict protocol to outline the regions. From the multiple delineations a surrogate for ground truth was derived in a form of a fuzzy set. In FIG. 24, assessment of the agreement with the surrogate of ground truth of each of the nine delineation is shown. A discrepancy between the hand delineations that affected the resulting surrogate of ground truth was observed, due to using “experts” who were trained technicians showing noticeable intra and inter-operator differences.

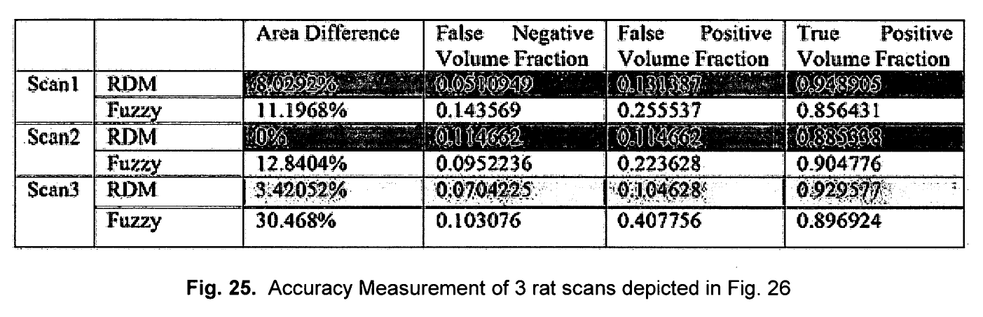

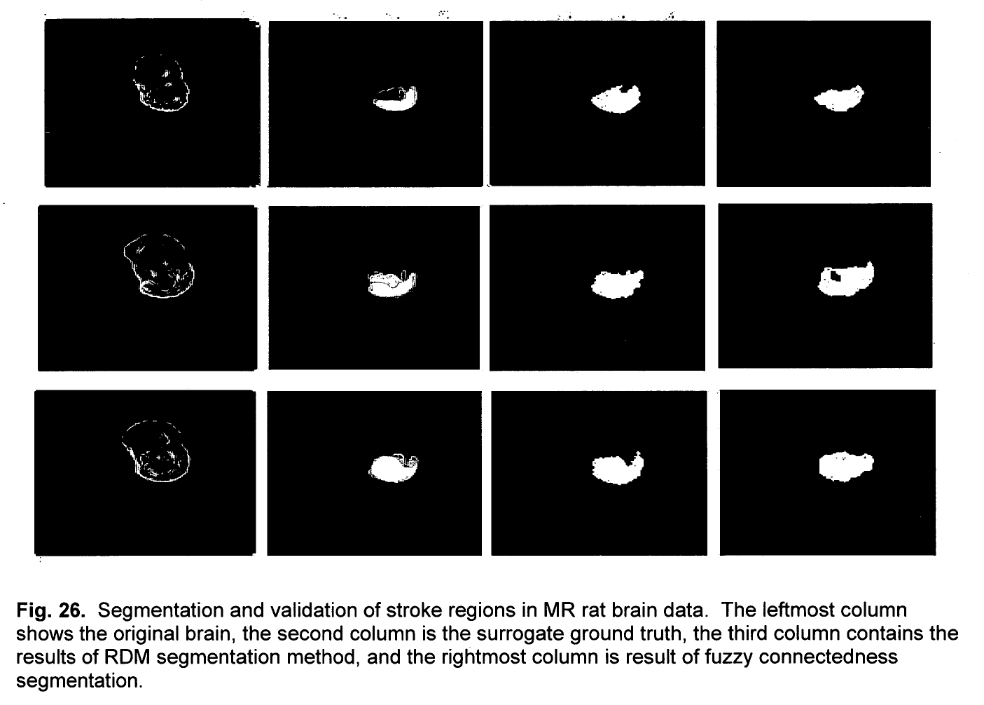

The disclosed symmetry based RDM segmentation tool classifies the whole brain into healthy and pathological regions as shown in column 3 of FIG. 24. We compare our segmentation results with corresponding previously segmented regions that were processed with fuzzy connectedness algorithm. In FIG. 25, accuracy measurements are compared for the disclosed RDM method and fuzzy connectedness using three rat brains, depicted in FIG. 24. With the RDM method, we showed an improvement in the TPVF metric in rat 1(scan 1) and rat 3 (scan 2), while a comparable TPVF in rat 2 (scan 2). Similarly FPVF and FNVF, generally show an improvement. Therefore, we saw a promise in the RDM method to be used in the future for rapid quantification of cerebral infarct volumes without the necessitating animal sacrifice. Having a surrogate of ground truth derived from hand delineations, FNVF, FPVF, TPVF parameters can be used to evaluate accuracy of segmentation.

A generic method to compute axis of symmetry and quantification of asymmetry in brain imagery is thus disclosed. There are various clinical applications that make use of brain imagery where quantification of asymmetry provides potential computer-assisted assessment and diagnostic tool: e.g. objective quantification of perfusion-weighted computed tomography in subarachnoid hemorrhage; quantification of previously undetected silent infarcts on MR-perfusion in patient following carotid endarterectomy; pre-segmentation of ischemic stroke regions in a rat model of temporary middle cerebral artery occlusion; quantification of diffusion-weighted images and apparent diffusion coefficient maps in the detection of acute strokes.

V. Automatic Correction of the 3D Orientation of the Brain Imagery

Detection and classification of human brain pathologies can be guided by the estimation of the departure of 3D internal structures from the normal bilateral symmetry. This comparison is useful since the human brain presents high level of bilateral symmetry that is partially absent under (asymmetric) pathological conditions. Symmetry is used by both clinical experts and automatic diagnostic systems to detect qualitatively asymmetric pattern indicating a wide range of pathologies (tumor, stroke, bleeding). However symmetry based analysis is hindered when the 3D brain orientation is misaligned, a common occurrence in clinical practice, since the misalignment causes the plane to intersect the symmetric structures at different heights. For neuroradiologists and computer program to correctly assess the pathological asymmetries it is crucial that the neural scans be registered to the coordinate system of the scanner. Lack of such registration may result in the images being in-homologous within the same coronal or axial level.

The detection and computation of a symmetry plane is a necessary step in symmetry-based analysis of neural images. Assessment by either a clinical expert and/or automatic system based on symmetry analysis (for example, using a the Relative Difference Map (RDM) quantification as described above) must account for the realignment of the brain to the scanner coordinate system.

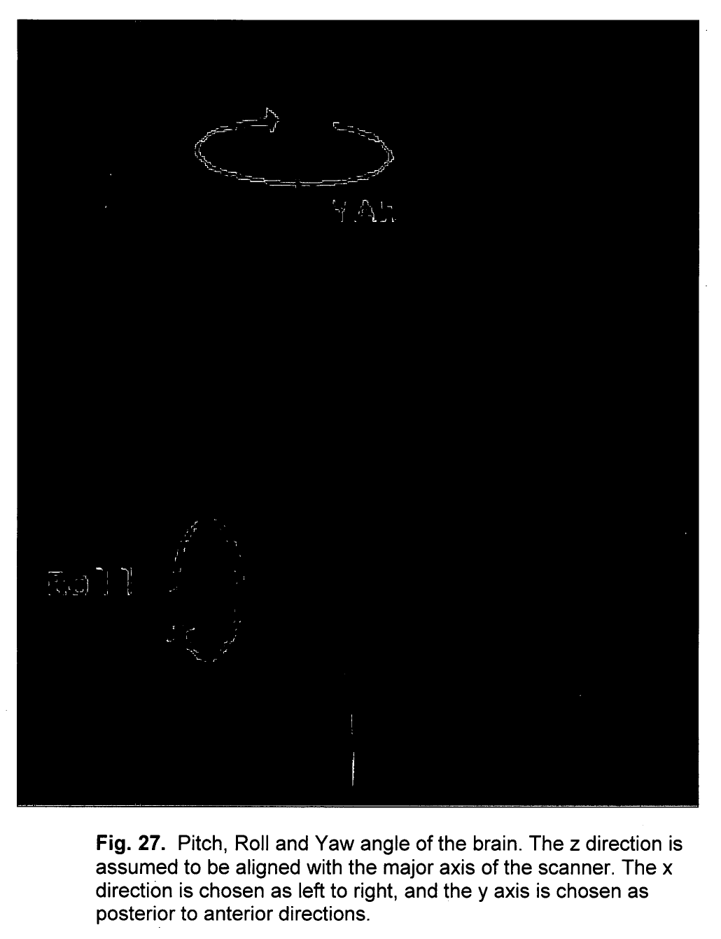

It is noted that the normal brain exhibits approximate bilateral symmetry with respect to mid-sagittal plane and it is thus useful to define the mid-sagittal plane as the plane that best separates the brain into two bi-fold mirror hemispheres. Approaches to estimate the mid-sagittal plane can be placed into two major categories: 2D based methods; and 3D based methods. 2D methods that are applied to individual 2D scans, most always can be generalized to 3D cases. For example, Brummer proposes a method of using the Hough transform to identify cerebral inter-hemispheric fissure, and Marais extracts the fissure using snakes, and uses an orthogonal regression from a set of control points. The method presented by Liu (see references below for the sources describing the Brummer, Marais and Liu proposals), sequentially deals with 2D slices for finding symmetry axis for each coronal or axial slice, and then computes a 3D plane from set of these lines. Because these methods process brain volume slice-by-slice, the global symmetry of the whole brain may not be captured. In case where the head is tilted along y axes, as in FIG. 27, a structure displayed in the same axial slice will not reside in the same plane and the symmetry axes computed independently in each slice will thus produce flawed results.

In 3D based approaches the plane that maximizes the bilateral symmetry is captured. Prima et al compute local similarity measures between two sides of the brain, using a block matching procedure. Ardekani conducts iterative search on the unit sphere, in order to find the plane with respect to which the image exhibits maximum symmetry (see references below). These algorithms that are based on local search reduce the amount of computation, but fail on clinical brain images with gross asymmetries often caused by pathological conditions.

Thus, in exemplary embodiments of the present invention, a technique to automatically identify the symmetry plane and correct the 3D orientation of volumetric brain images in a cost effective way can be used. It is a 3D based method incorporating head extraction, principal component analysis and re-interpolation. As background, the following assumptions, are made:
Any plane of symmetry in a body is orthogonal to a principal axis, and
Any axis of symmetry in a body is a principal axis.

A principal axis based method can potentially fail to solve the problem of brain symmetry because, abnormal asymmetries distort the underlying symmetry of the brain. However, it is felt that such pathological asymmetries only distort local symmetry, without altering the global geometry of the rigid body. Therefore, the 3D orientation of brain should remain unaffected. Thus, treating the head as a solid 3D object, a robust method to compute successfully the plane of symmetry, and making it resistant any specificity of brain internal geometry, can be implemented.

Algorithm Overview

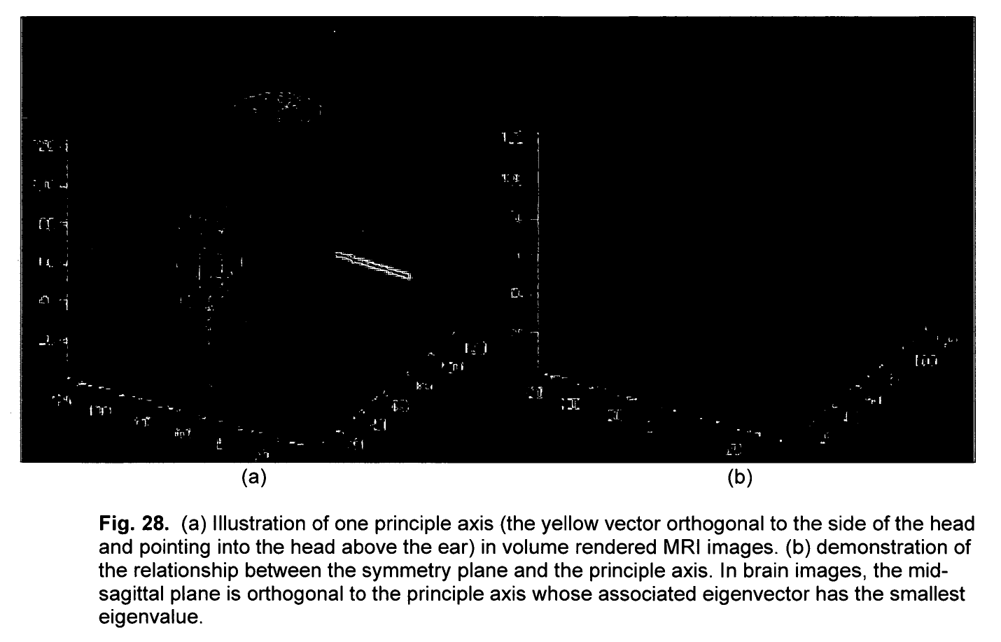

An idealized situation is assumed, with the diameter of all three principal axes of the human brain to be distinctive, and that a set of slices representing a complete head volume are available. Thus, neither the top of the head is truncated, nor too large sections of the neck and shoulder are present, as shown, for example, in FIG. 28. Under such assumptions, an exemplary algorithm comprises:

Step 1) subtract the background from the image and make the tilted volume V₁ as a binary volume B₁. Then remove all slices that do not enclose head-only (or including the neck and shoulders if applicable). By doing this, it is guaranteed that binary volume B₁ is a 3 dimensional ellipsoid-like shape, where there exists 3 distinctive principal axes;

$${B}_{t} ( x , y , z ) = ( \begin{matrix}1 , if & {V}_{1} ( x , y , z ) > k \\ 0 , if & {V}_{1} ( x , y , z ) \leq k ⋃ {V}_{1} ( x , y , z ) ∉ H\end{matrix} ) ,$$

where k is a scalar value that can be assigned a small value assuming the background intensity is zero. Then, the object (head) can be extracted from the background. H is the set of slices representing head-only region, without the neck and shoulder structures. H={All the head voxels};
Step 2)—resample, with a higher resolution (up-sampling) the tilted volume Vₜ, in vertical axis, to make each voxel a cubic.

Let the original image matrix size be NₓxN_{y}xN_{z} voxels, and thus the dimension of each voxel is dₓd_{y}xd_{z}. We know that dₓ=d_{y}, d_{z}=βdₓ where β is a constant double value that denotes the ratio between the height of the voxel in z direction and its horizontal dimension. The new dimension in z can therefore be written as N_{z}′ where N_{z}′=(d_{z}/dₓ)×N_{z}=βN_{z} such that each voxel dimension d_{z}′=(d_{z}/β)=dₓ=d_{y}.

We interpolate (β−1)N_{z}×Nₓ×N_{y} points from the existing sample N_{z}×Nₓ×N_{y} points. We apply cubic B spline interpolation since it provides high order estimation of the parameters to generate better visual appearance. Using this process, a series of unique cubic polynomials can be fitted between each of the data points, with the expectation that the curve obtained be continuous and appear smooth.

We resample again, the volumetric data Bₜ in x, y, z axis, with a lower resolution (down-sampling), to reduce the computational throughput while maintaining the closest solution to the optimum. The new dimension of the stack image slices is defined as N̅_̅{̅z̅}̅× N̅ₓ̅× N̅_̅{̅y̅}̅=(1/u)N_{z}′×Nx×Ny, where u is an integer, u>1, that preserves equally spaced samples in x, y and z dimensions every u pixels.

Step 3) The sampled object inside the sphere is processed by the algorithm that solves the eigenvalue problem associated with the object's inertia matrix. Firstly, we compute the centroid of the brain. Let the centroid of the binary Volume B₁(x, y, z) be represented by (x_{g}, y_{g}, z_{g})^{T} where

$${x}_{g} = \frac{{\sum }_{x , y , z} x {B}_{t} ( x , y , z )}{{\sum }_{x , y , z} {B}_{t} ( x , y , z )}$$

$${y}_{g} = \frac{{\sum }_{x , y , z} y {B}_{t} ( x , y , z )}{{\sum }_{x , y , z} {B}_{t} ( x , y , z )}$$

$${z}_{g} = \frac{{\sum }_{x , y , z} z {B}_{t} ( x , y , z )}{{\sum }_{x , y , z} {B}_{t} ( x , y , z )}$$

We compute the covariance matrix I of the volumetric binary image Bₜ(x,y,z). The covariance matrix I can be derived from the second-order central moments as follows:

$$I = [ \begin{matrix}{I}_{xx} & - {I}_{xy} & - {I}_{xz} \\ - {I}_{yx} & {I}_{yy} & {I}_{yz} \\ - {I}_{zx} & - {I}_{zy} & {I}_{zz}\end{matrix} ]$$

$$where {I}_{xx} = {\sum }_{x , y , z} [ {( y - {y}_{g} )}^{2} + {( z - {z}_{g} )}^{2} ] {B}_{t} ( x , y , z )$$

$${I}_{yy} = {\sum }_{x , y , z} [ {( x - {x}_{g} )}^{2} + {( z - {z}_{g} )}^{2} ] {B}_{t} ( x , y , z )$$

$${I}_{zz} = {\sum }_{x , y , z} [ {( x - {x}_{g} )}^{2} + {( y - {y}_{g} )}^{2} ] {B}_{t} ( x , y , z )$$

$${I}_{xy} = {I}_{yx} = {\sum }_{x , y , z} [ ( x - {x}_{g} ) ( y - {y}_{g} ) ] {B}_{t} ( x , y , z )$$

$${I}_{xz} = {I}_{zx} = {\sum }_{x , y , z} [ ( x - {x}_{g} ) ( z - {z}_{g} ) ] {B}_{t} ( x , y , z ) \\ {I}_{zy} = {I}_{zy} = {\sum }_{x , y , z} [ ( y - {y}_{g} ) ( z - {z}_{g} ) {B}_{t} ( x , y , z )$$

The inertia matrix J can be formed from covariance matrix I, J=trace(I)I₀−I where I₀ is the 3×3 identity matrix. The three eigenvectors of J are the principal axes, which are mutually orthogonal to each other. The centroid and the principal axes completely describe the orientation of a volume at an arbitrary orientation. The eigenvector corresponding to the smallest eigenvalue has the direction that is orthogonal to the mid-sagittal plane. Therefore, by computing the angle of this eigenvector with respect to the x, y and z axes, we actually acquire rotational angles (yaw, roll and pitch) of the mid-sagittal plane, as shown in FIG. 27.

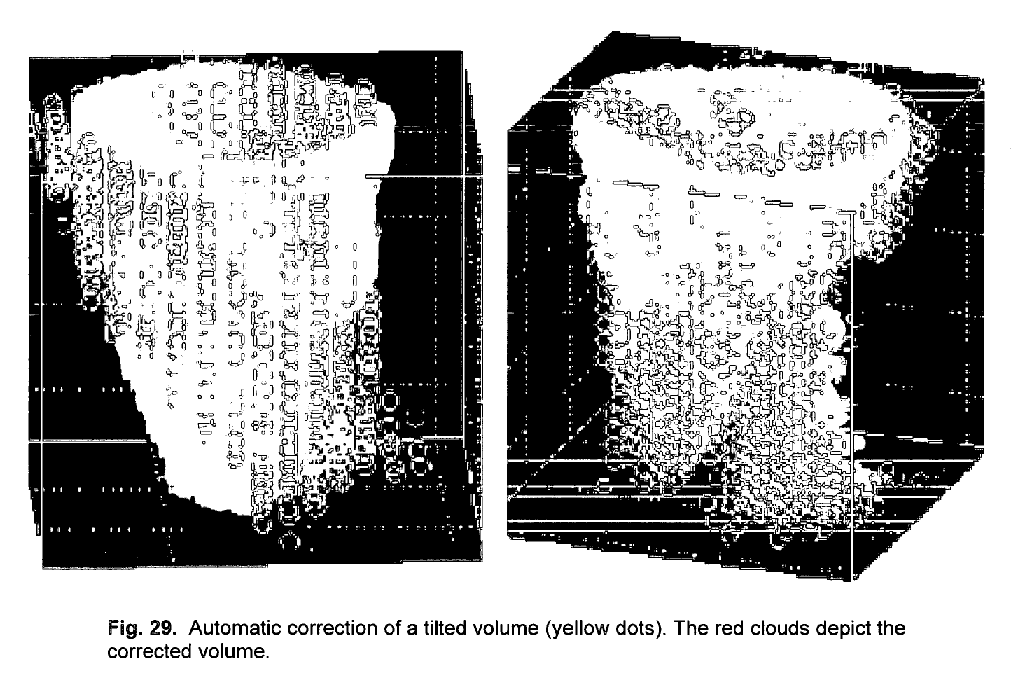

Step 4) Affine spatial transformation for tilt correction after the principal axes have been derived. let R=RₐR_{β}R_{γ} represent the rotation matrix as

$$\begin{matrix}{R}_{a} {R}_{β} {R}_{γ} = [ \begin{matrix}cos ( α ) & sin ( α ) & 0 \\ - sin ( α ) & cos ( α ) & 0 \\ 0 & 0 & 1\end{matrix} ] [ \begin{matrix}cos ( β ) & 0 & - sin ( β ) \\ 0 & 1 & 0 \\ sin ( β ) & 0 & cos ( β )\end{matrix} ] \\ [ \begin{matrix}1 & 0 & 0 \\ 0 & cos ( γ ) & sin ( γ ) \\ 0 & - sin ( γ ) & cos ( γ )\end{matrix} ]\end{matrix}$$

where α, β, γ are the rotation angles with respect to the x, y and z axes (Yaw, Roll, Pitch), respectively. Let T represent the translation matrix.

$$T = [ \begin{matrix}1 & 0 & 0 & 0 \\ 0 & 1 & 0 & 0 \\ 0 & 0 & 1 & 0 \\ σ x & σ y & σ z & 1\end{matrix} ]$$

where sx, sy, sz are the offset from the centroid of the head to the center of the scanner coordinates. V_{c}=Vₜ×R×T where Vₜ is the original input volume. V_{c} is the 3D corrected volume re-centered at the centroid (x_{g}, y_{g}, z_{g})^{T}. Re-slicing is conducted on the re-centered and re-tilted volume Vc. Again, we implement cubic spline interpolation to achieve better smoothness and higher precision, as shown in FIG. 29.

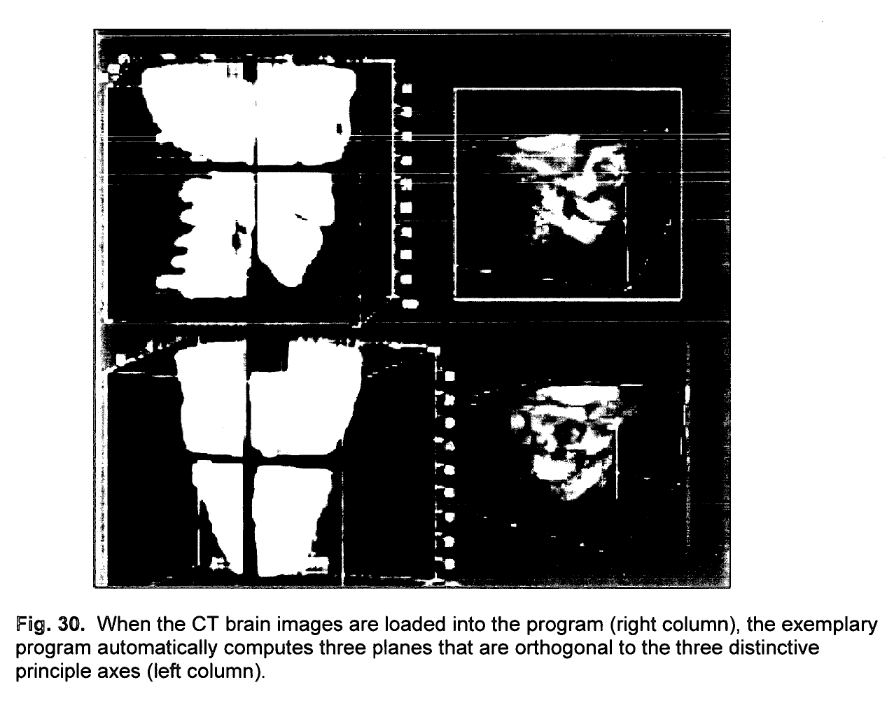

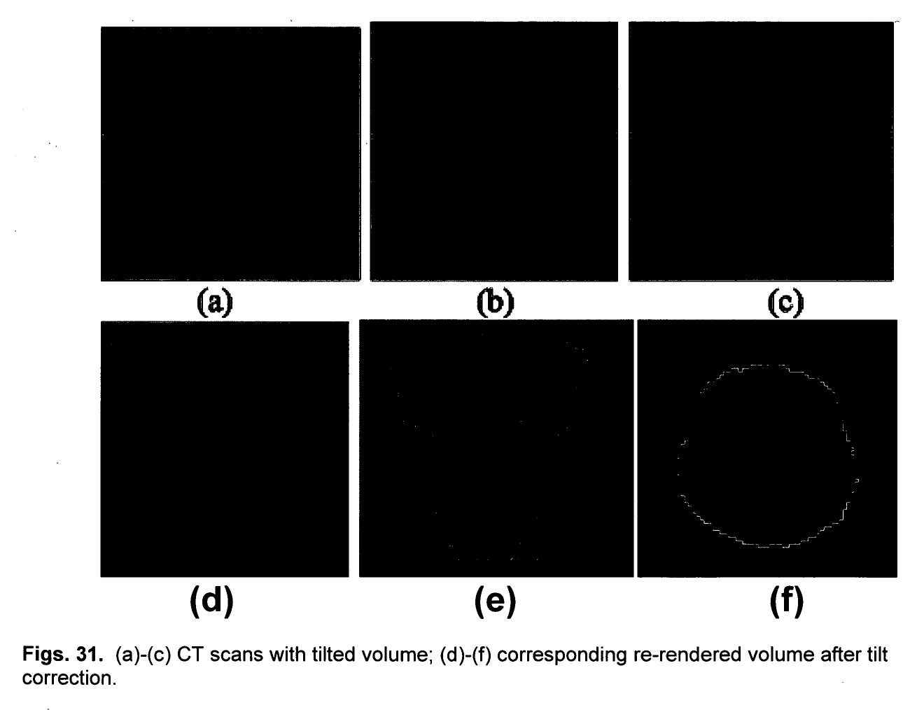

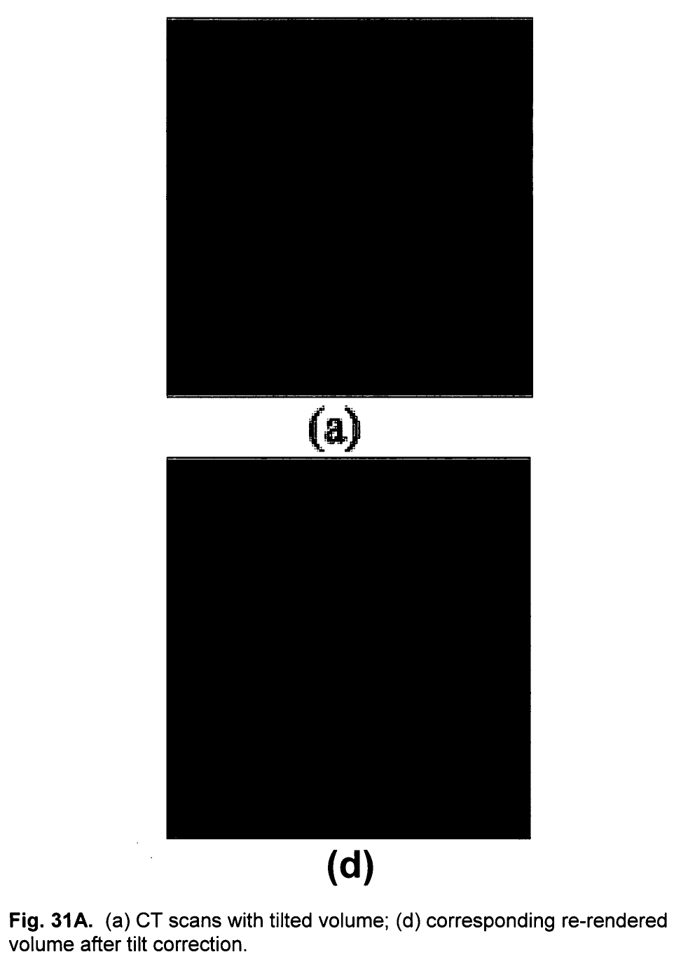

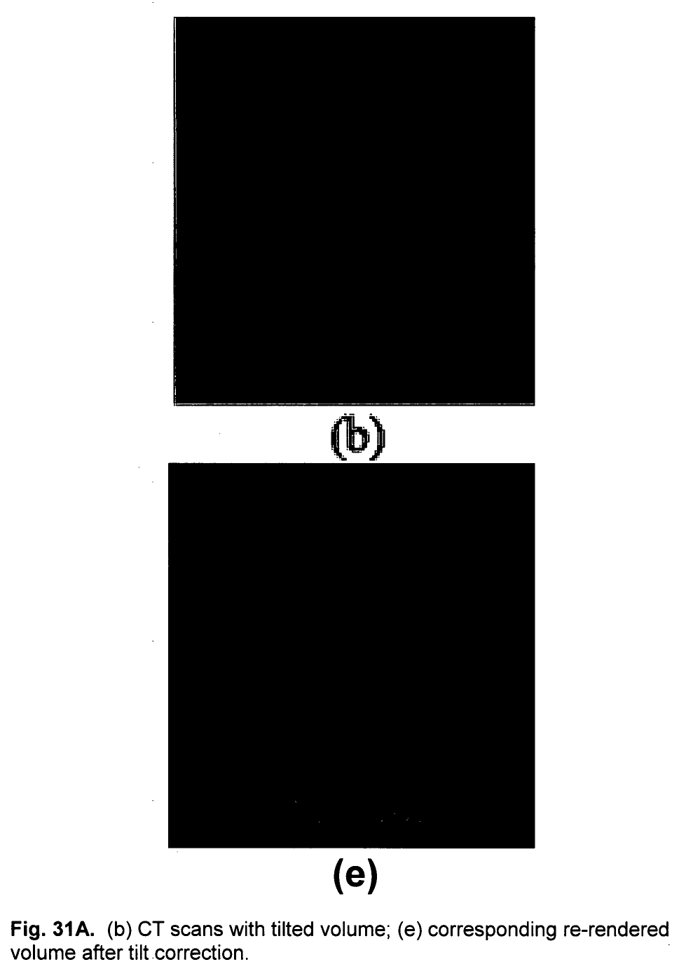

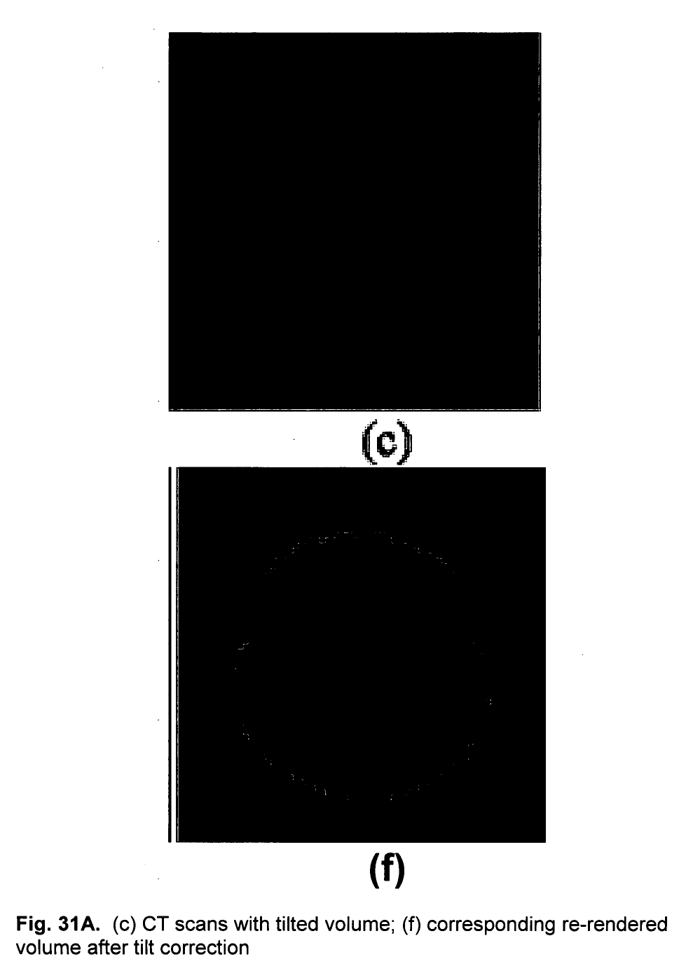

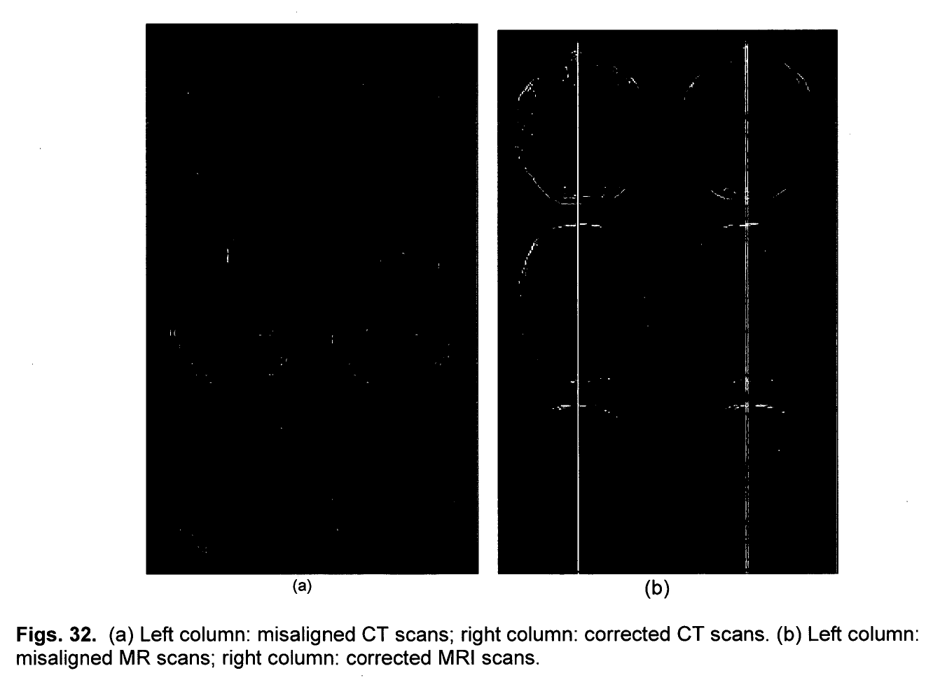

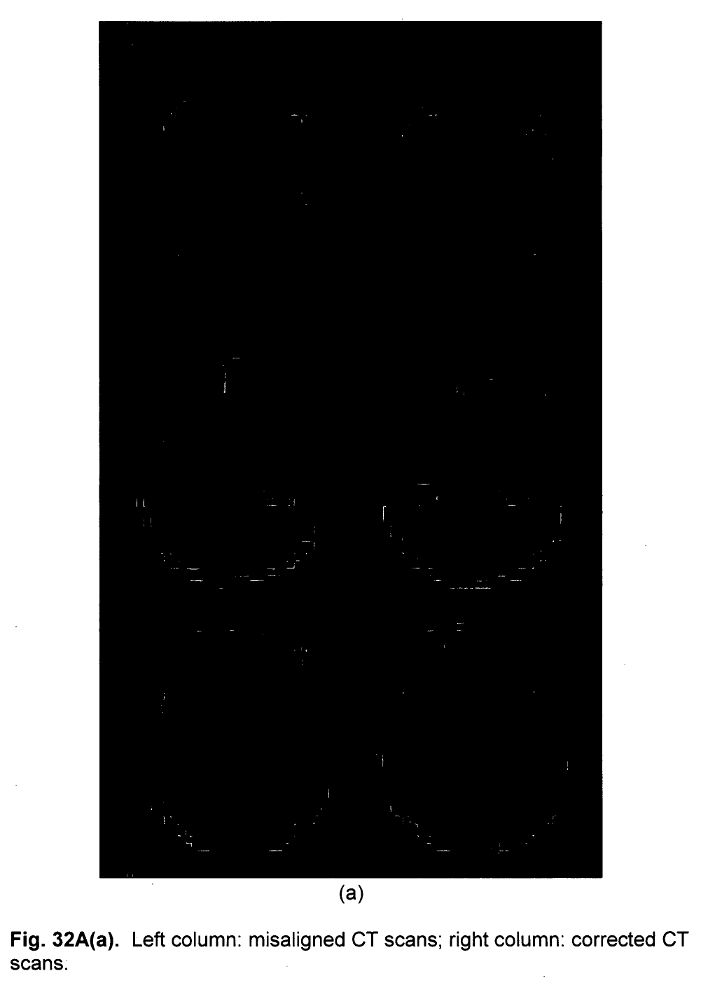

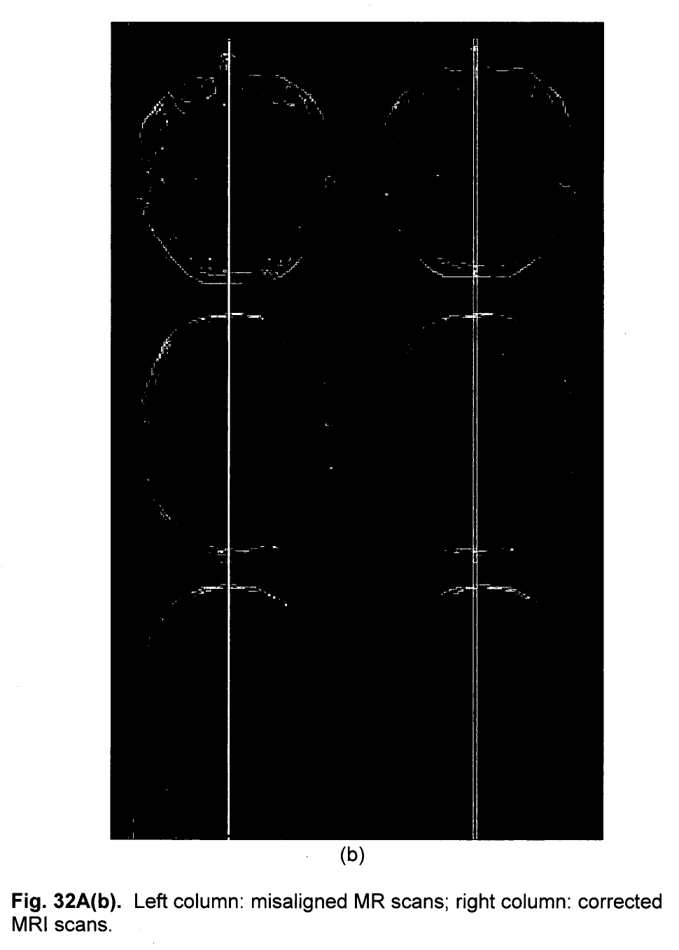

FIGS. 30-32 illustrate the method on different examples. In these examples the method was applied to brain MRI images from 15 patients selected from the pool of PACS (picture archiving and communication system) image database at Columbia Presbyterian Hospital. The dataset is composed of a mixture of T1-weighted MRI scans and T2-weighted MRI scans, half of which have distinctive brain lesion and certain level of brain shift. Each scan is represented by a matrix of dimension 256×256×124, with voxel dimension 1.01×1.01×2.0 mm³.

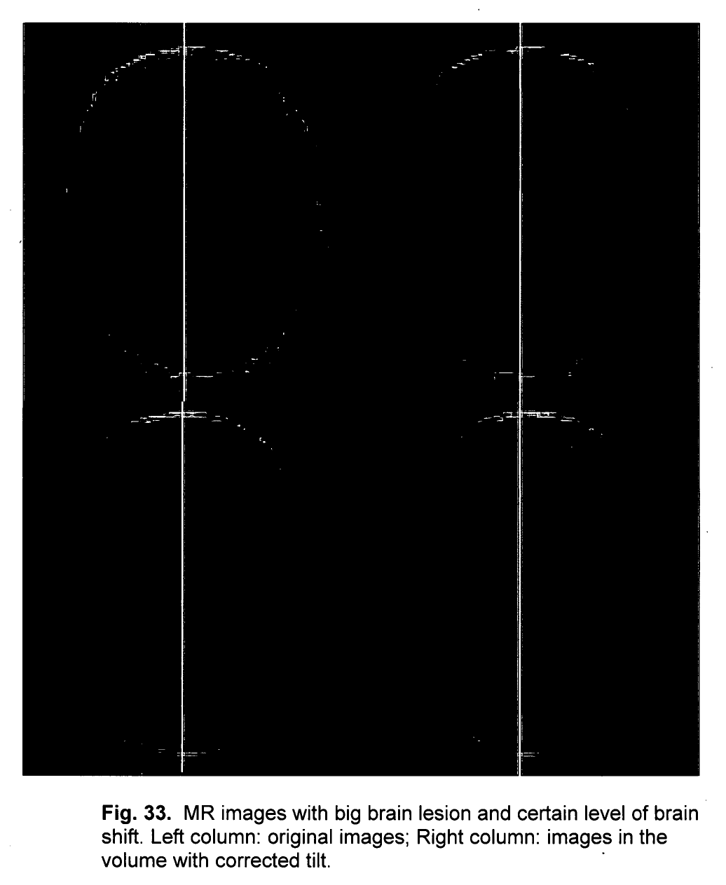

For each patient, two image volumes are generated, one that has matrix dimension of 256×256×248, and the other of 128×128×124. A total of 30 image volumes were generated and qualitatively assessed by a physician. Out of 30, 29 cases were judged to be highly accurate in terms of head tilt correction and re-interpolation of the brain along the z-axis. A prospective study for quantitative validation is currently being designed. One set of results has been illustrated in FIG. 33, this patient has a large brainstem lesion with ventricular effacement and left-to-right shift. However, the local asymmetry doesn't modify the global orientation of the brain. The result shows that we can still precisely capture the symmetry plane regardless of the local pathological conditions.

Thus, an automatic, cost-effective method to automatically correct the tilt of the brain in 3D space is presented. The technique provides a new tool for symmetry based analysis of the brain pathologies, which present as asymmetric features (e.g., tumor, stroke, bleeding). Preliminary results for MR and CT brain scans have shown promise and further studies to validate the method are ongoing. As was demonstrated, the technique performs robustly even in the presence of acquisition noise, bias field and pathological asymmetry because the method exams the global geometrical property of the head by the principle component analysis. When the data is truncated or the field of view is neck/shoulder inclusive, the assumption that the head is ellipsoid-like 3D object is not met and the technique may fail. Failure of the technique in these cases can be avoided by the correct inclusion or exclusion of an object of interest.

VI. Symmetry Identification Using Partial Surface Matching and Tilt Correction in 3D Brain Images

The human brain exhibits a high level of bilateral symmetry, although it is not perfectly symmetrical. Detection and computation of symmetry plane in the brain has many applications. Symmetry is used by clinical experts to detect qualitatively asymmetric pattern indicating a wide range of pathologies. Similarly computer aided systems are used to quantify asymmetry and to automatically generate hints for clinicians. Since neuroradiologists use routinely symmetry in their assessment of brain images, the misalignment of the patient's head in the scanner often leads to false clinical interpretation of the patients scans. Likewise, for computer program to correctly assess the pathological asymmetries it is crucial that the neural scans are not tilted but correctly aligned and oriented within the coordinate system of the scanner. However, the tilt of the head that is observed in practice quite often, not always can be controlled. It may be caused by the immobility of the patient, inexperience of the technician, or the imaging device itself. This makes radiological slices of the brain images no longer homologous within the same coronal or axial level. Thus, both assessment either by a clinical expert and/or automatic system based on symmetry analysis, like one similar to the Relative Difference Map (RDM) quantification, first require the brain to be re-aligned within the scanner coordinate system.

There are two major approaches to solve the problem of computing the symmetry plane in brain images. One is the 2D based method, and the other is the volume based method. The number of reported methods to compute brain symmetry, in 3D volume is considerably smaller than those in 2D. The planar method that is applied to 2D radiological brain slices, may not always be extendable to 3D cases. For example, Brummer proposes a method of using the Hough transform to identify cerebral inter-hemisphereic fissure. Marais extracts the fissure using snakes, and uses an orthogonal regression from a set of control points. The method presented by Liu, estimates 2D mid-sagittal axis for each coronal or axial slice, and then computes a 3D plane from set of these lines. Because these methods process brain volume slice-by-slice, the global symmetry of the whole brain is not captured. In case where the head is tilted along the axis from posterior to anterior, a structure displayed in the same axial slice will not reside in the same plane and the symmetry axes computed independently in each slice will produce flawed final result. In 3D approaches, the plane that maximizes the bilateral symmetry is captured. Prima et al computes local similarity measures between two sides of the brain, using block matching procedure. Minovic proposed using principle axes to characterize symmetry plane, and, Sun extended Minovic's work and developed an algorithm for finding symmetry planes of 3D objects using extended Gaussian image representation. However both methods only focused on synthesized object and may be applicable if the clinical brain images have truncated field of view. Ardekani conducts iterative search on the unit sphere, in order to find the plane with respect to which the image exhibits maximum symmetry. These algorithms that are based on local search reduce the amount of computation, but fail on clinical brain images with gross asymmetries often caused by pathological conditions.

Described below is a method to automatically compute the symmetry plane and correct the 3D orientation of patient brain images. Similar to most existing 3D approaches, the mid-sagittal plane is defined as the one that maximizes the similarity between two halves of the brain. Therefore the problem decomposes into searching over a set of possible planes to achieve the maximum of similarity measure between the original image and its reflection. When the 3-D volume is taken into account, the robustness is difficult to achieve with global criteria that is affected by the strong underling asymmetry. For most existing 3D methods, apart from their sensitivity to pathological asymmetries, another common drawback is the computational cost due to the optimization scheme when searching through the set of possible planes.

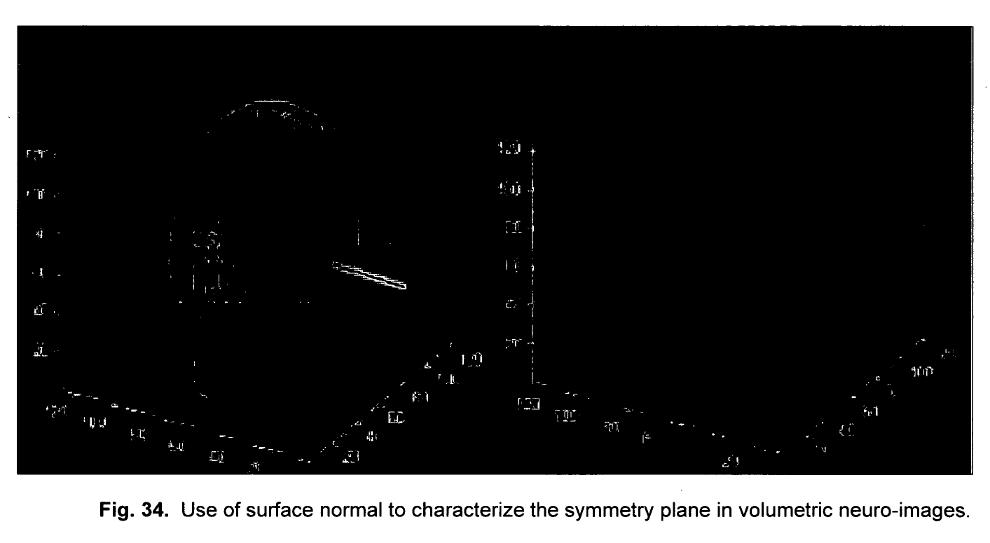

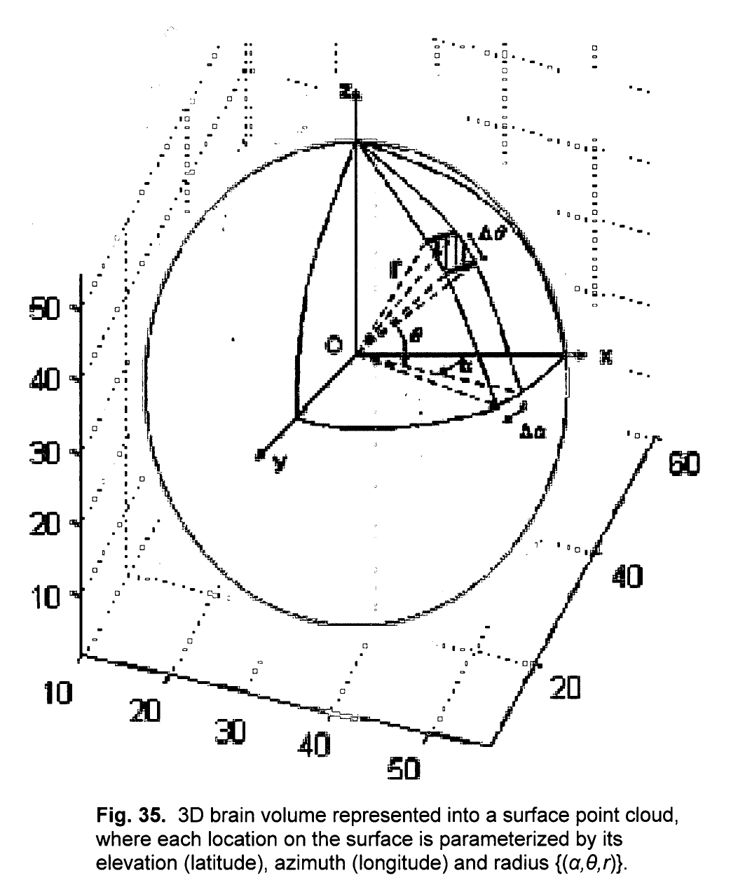

Different from those 3D approaches, we choose the surface normal to characterize the geometry of the symmetry planes in 3D Euclidian space, as shown in FIG. 34. Instead of using intensities or edges, for example, as the characteristic features, the similarity criterion is computed from the point cloud on the surface of the brain., regardless the contents inside. We therefore transform the 3D brain volume into a thin point cloud and each location on the surface has been parameterized by its elevation (latitude), azimuth (longitude) and radius, as depicted in FIG. 35. The removal of the interior contents of the brain makes this approach perform robustly in the presence of the brain pathologies, e.g. tumor, stroke and bleed.

After re-parameterization, we decompose the symmetry computation problem into a surface matching routine. The search for the best matching surface patches is performed utilizing a multi-resolution approach which decreases computational time considerably.

Lastly, spatial affine transform is performed to rotate the 3D brain images and align them within the coordinate system of the scanner. The corrected brain volume is re-sliced such that each planar image represents the brain at the same axial level. The algorithm is 3-D and is insensitive to acquisition noise, bias field and pathological asymmetries and the incomplete field of view. In the following sections, the algorithm is detailed.

Data Representation

Assuming that there is a single object (the head) of interest in a volumetric dataset, it is further assumed that patient scans do not suffer from skull or skin lesion so that the surface of the head is complete and almost symmetrical. Given a region of interest R within an image I, δ is the background cutoff that separates background from the head. We assign a very small value to δ since the background intensity in most image modalities is close to zero. X_{R}, the characteristic function can be found as follows:

$${X}_{R} ( x , y , z ) = \{ \begin{matrix}1 if ( x , y , z ) ∈ R : I ( x , y , x ) > δ & \\ 0 & otherwise \end{matrix}$$

With the integration over the whole image I, the volume A can be defined as the 0th moment of R.
A=∫∫∫ₜX_{R}(x,y,z)dxdydz=∫∫∫_{R}dxdydz
Then the centroid C=( x̅, y̅, z̅) can be is computed as
x̅=(∫∫∫_{R}xdxdydz)/A;
y̅=(∫∫∫_{R}ydxdydz)/A;
z̅=(∫∫∫_{R}zdxdydz)/A;

Although it is possible that X_{R} will assign some inner structures that present low intensities to be zero, but this won't affect the correct computation of the centroid. The technique extracts the points on the surface of the brain and maps them onto spherical polar coordinates. We define a to be the azimuthal angle in the x y-plane from the x axis with 0≦α<2π, and define θ to be the polar angle from the z-axis with −π/2≦θ<π/2, as shown in FIG. 35. θ is allowed to zero at the equator, and 0≦θ<π/2 for points from the equator to the north pole. The algorithm allows rays {r(α,θ)} emanating from the centroid C to the boundary elements of X_{R}. The intersections of the ray with the furthest boundary of the region entail the sampled surface point cloud of the brain. Every point on such a point cloud {(x,y,z)} has been uniquely mapped to a triple set {(α,θ,r)} where the radius r is the distance from a point to the centroid. It is noted that α is also called azimuth (longitude ) and θ elevation (latitude).

2. Geometry of the Mid-Sagittal Plane (MSP)

Under Cartesian coordinates the mid-sagittal plane can be represented as
aX+bY+cZ+d=0
where N=(a, b, c) is the normal vector that is perpendicular to the mid-sagittal plane, and d/\sqrt{a²+b²+c²} is the perpendicular distance of the plane from the origin. Each plane is characterized by a unique set of parameters (a, b, c). Our aim was to find the triplet (a, b, c) of a symmetry plane with respect to which the image I exhibits maximum “symmetry” measure. Any parameter set (a, b, c) in Cartesian coordinates has unique counterpart in spherical polar coordinates. This relationship is demonstrated as follows:
a=R cos(θ_{N})cos(α_{N});
b=R cos(θ_{N})sin(α_{N});
c=R sin(θ_{N});
d=−N·C

Given a unit normal vector orthogonal to the symmetry plane where R equals to 1, the symmetry plane can be characterized as N (a, b, c)=N (θ_{N}, α_{N}) where
N(θ_{N},α_{N})=((cos(θ_{N})cos(α_{N}), cos(θ_{N}) sin(α_{N}), sin(θ_{N}))^{T}
Therefore, what is essentially sought is the normal vector N(θ_{N},α_{N}) that characterizes the symmetry plane from our re-parameterized searching space until the maximum of similarity measure has been achieved.
3. Sampling Strategy and Constrained Search

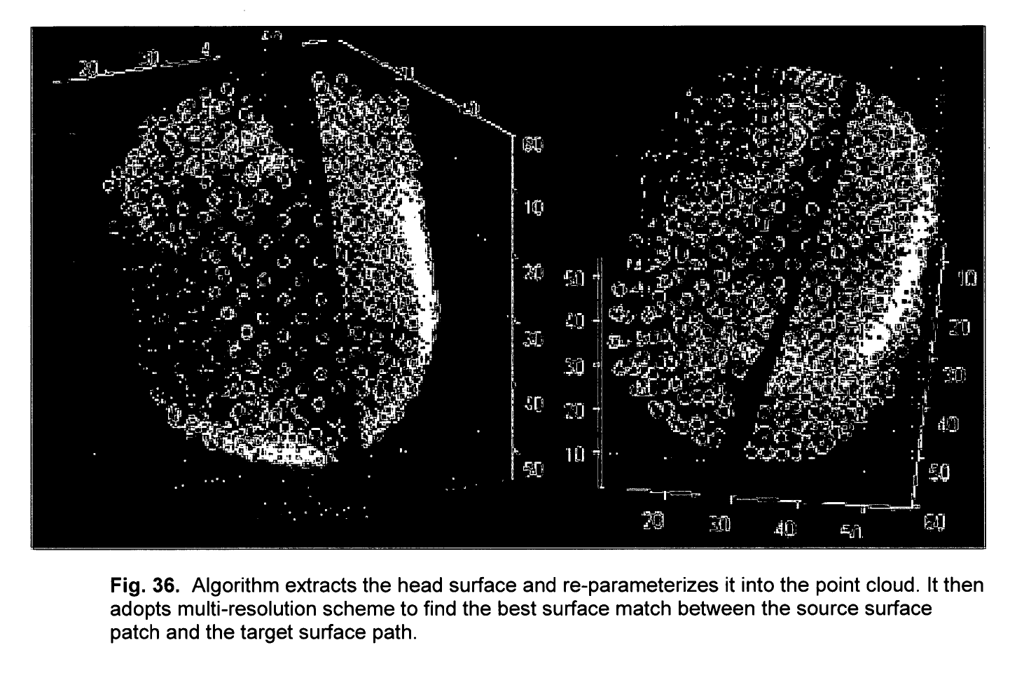

The rays can be quantized by the discrete approximation to θ=kₜΔθ, α=k₂Δα, k1, k2εZ and the step size (Δθ,Δα) defines the sampling resolutions, as seen in FIG. 36.

The procedure is to select q points on the point cloud and evaluate the symmetry measure with respect to the opposing q points on the other side of the brain. The initial guess of the normal vector of the symmetry plane is θ_{N}=0,α_{N}=0, a vector directing from the centroid to the right ear. This initial guess was iteratively refined until convergence. A surface patch S({θᵢ}, {αₜ}) is defined, where its θ spans between the range [θ_{N}−k₁Δθ, θ_{N}+k₁Δθ]and a spans between [α_{N}−k₂Δα, α_{N}+k₂Δα]. In a primary study, we let each surface patch θ spans 60 degrees and a span 120 degrees so that it forms a roughly trapezoid shape. We set the sampling resolution to be Δθ=Δα=3°.

This gives rise to a surface patch quantized by a discrete points cloud, as shown in FIG. 36. This initial surface patch S({θᵢ}, {αᵢ}) should be centered around the first guess of the normal vector. We call this source surface patch (shown as the yellow cloud (right side of each image) in FIG. 36).

The search for the target surface patch (shown as the blue cloud (left side of each image) in FIG. 36) SR({θᵢ}, {αᵢ}), is the procedure to find the best matching counterpart of S({θᵢ}, {αᵢ}) on the other side of the brain. Starting from the opposite direction v(θ_{R}α_{R}), θ_{R}=θ_{N},α_{R}=α_{N}+Π the algorithm examines all the candidate surface patches by evaluating similarity measure in the vicinity about the vector v(θ_{R}, α_{R}) within the range (θ_{R}±pΔ_{θ}, α_{R}±pΔ_{α})pεZ, where (Δ_{θ},Δₐ) is the sampling interval between adjacent steps. Total 4p² searching steps is conducted on 4 p² potential candidate surfaces within one iteration. SR({θᵢ}, {αᵢ}) should thus span the same surface area as S({θᵢ}, {αᵢ}). The optimum finding of v(θ_{R},α_{R}) enters into the next iteration.

Similarity Measure

For each iteration, we searched over 4p² possible surfaces until we achieved the maximum of similarity measure between the source surface patch S({θᵢ}, {αᵢ}) and target surface patch S({θᵢ}, {αᵢ}). Correlation coefficient (CC) was chosen as the similarity measure. We considered all the radii of from surface S({θᵢ}, {αᵢ}) as x₁,x₂, . . . ,xₙ and all the radii from surface SR({θᵢ}, {αᵢ}) as y₁, y₂, y₃. The correlation coefficient between S and SR is:

$$CC = \frac{\frac{1}{n} {\sum }_{i = 1}^{n} ( {x}_{i} - {x}_{mean} ) ( {y}_{i} - {y}_{mean} )}{\frac{1}{n} \sqrt{{\sum }_{i = 1}^{n}} {( {x}_{i} - {x}_{mean} )}^{2} {\sum }_{i = 1}^{n} {( {y}_{i} - {y}_{mean} )}^{2}}$$

where −1≦CC≦1, The CC measures the strength of the linear relationship between S and SR. Our technique seeks the highest absolute value of CC, the one closest to 1, which represents the strongest correlation between source and target surface patches.
Multi-Resolution Scheme

By adopting a coarse resolution search at larger step intervals (Δθ,Δα), we achieved a rough estimation of the normal vector N(θ_{N},α_{N}) at the initial runs. As iteration evolves, we reduced the search space (pΔ_{θ},pΔ_{α}) by applying smaller p, while increasing the sampling resolution by shortening step size (Δ_{θ},Δ_{α}). Our initial search space spanned pΔ₀=pΔ_{α}=40°, with sampling interval 2 degrees. We refine the search resolution by the factor of 2 at each iteration, so that at the second run, the search space spanned 20 degrees at 1 degree intervals, so on and so forth for subsequent iterations.

The process was repeated at finer resolution proceeding from the optimum SRᵢ found by the previous iteration. The final finding of the normal vector N(θ_{N},α_{N}) was determined by the coefficients computed from the optimum matching surfaces.

4. Affine Spatial Transformation for Tilt Correction.

Let R₀ represent the rotation matrix as

$${R}_{0} = {R}_{ω} {R}_{β} {R}_{γ} = \\ [ \begin{matrix}cos ( ω ) & sin ( ω ) & 0 \\ - sin ( ω ) & cos ( ω ) & 0 \\ 0 & 0 & 1\end{matrix} ] [ \begin{matrix}cos ( β ) & 0 & - sin ( β ) \\ 0 & 1 & 0 \\ sin ( β ) & 0 & cos ( β )\end{matrix} ] [ \begin{matrix}1 & 0 & 0 \\ 0 & cos ( γ ) & sin ( γ ) \\ 0 & - sin ( γ ) & cos ( γ )\end{matrix} ]$$

where ω,β,γ are the rotation angles with respect to the x, y and z axes (yaw, roll, pitch), respectively. In this case, ω=0, β=α_{N}, γ=θ_{N}. Let T represent the translation matrix. V_{c}=Vᵢ RₒT where Vₜ is the original input volume and V_{c} is the 3D corrected volume recentered at the centroid. The corrected brain volume was re-sliced such that each planar image represents the brain at the same axial level. We implemented cubic spline interpolation to achieve better smoothness and higher precision.
Exemplary Results

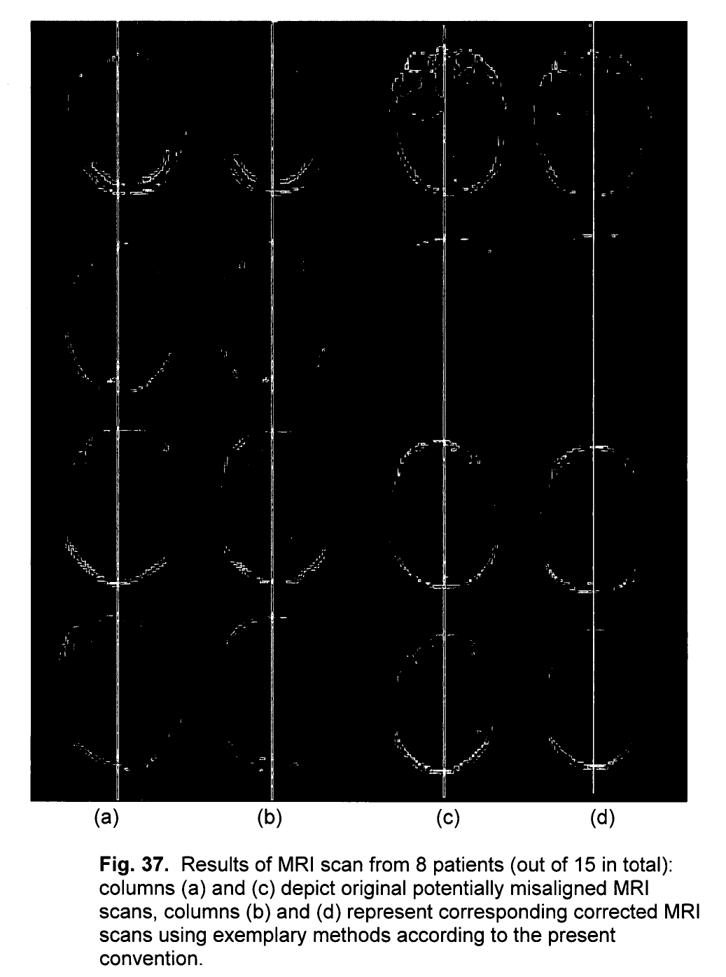

In FIG. 37, the results obtained on exemplary MRI volume images are illustrated. We applied our method to brain MRI images from 15 patients selected from the pool of PACS (picture archiving and communication system) image database at Columbia Presbyterian Hospital. The dataset was composed of a mixture of T1-weighted MRI scans and T2-weighted MRI scans, half of which have big brain lesion and/or certain level of brain shift. Each scan was of matrix dimension 256×256×124, with voxel dimension 1.01×1.01×2.0 mm³. Therefore, a total of 15 image volumes were generated. Out of 15 cases, 14 were judged to be highly accurate in terms of head tilt correction and re-interpolation of the brain along the z-axis. Thus this method presents promising potentials to precisely capture the symmetry plane regardless of local pathological asymmetries and acquisition noise.

Thus, in exemplary embodiments of the present invention, a technique for the automatic detection of the mid-sagittal plane in arbitrarily oriented three dimensional brain images and correction the 3D orientation of patient brain images in a cost effective way can be used. The algorithm is independent of the imaging modality and it is insensitive to incompleteness of the data. Unlike many of the classical symmetry-based methods, pathological asymmetries can severely degrade the computation of the symmetry plane, our method uses parameterized surface points to estimate the best similarity measure, and therefore it performs robustly in the presence of the normal/pathological asymmetries inside the brain. The search evolves at each iteration in the parameter space from the coarse level with lower resolution to the fine level with higher resolution. The use of multi-resolution paradigm dramatically reduces the computational cost while still producing satisfactory results.

While the present invention has been described with reference to one or more exemplary embodiments thereof, it is not to be limited thereto and the appended claims are intended to be construed to encompass not only the specific forms and variants of the invention shown, but to further encompass such as may be devised by those skilled in the art without departing from the true scope of the invention.

### REFERENCES

Atam P. Dhawan, “Image registration”, book chapter in “Medical Image Analysis”, p 254-p 259, published by John Wiley & Sons, 2003.

Sylvain Prima, Sebastien Ourselin, and Nicholas Ayache, “Computation of the Mid-Sagittal Plane in 3-D Brain Images”, IEEE transaction on medical imaging, vol. 21, no. 2, February 2002.

Celina Imielinska, Xin Liu, Joel Rosiene et al., “Towards Objective Quantification of Perfusion-Weighted Computed Tomography in the Setting of Subarachnoid Hemorrhage: Quantification of Symmetry and Automated Delineation of Vascular Territories”, Journal of Academic Radiology, 2005.

M. E. Brummer, “Hough transform detection of the longitudinal fissure in tomographic head images,” IEEE Trans. Med. Imag., vol. 10, pp. 74-81, March 1991.

B. A. Ardekani, J. Kershaw, M. Braun, and I. Kanno, “Automatic detection of the mid-sagittal plane in 3-D brain images,” IEEE Trans. Med Imag., vol. 16, pp. 947-952, December 1997.

C. Sun and J. Sherrah, “3D symmetry detection using the extended gaussian image,” IEEE Trans. Pattern Anal. Machine Intell., vol. 19, pp. 164-168, February 1997.

Y. Liu, R. T. Collins, and W. E. Rothfus, “Automatic bilateral symmetry (midsagittal) plane extraction from pathological 3D neuroradiological images,” presented at the SPIE, International Symposium on Medical Imaging, San-Diego, California, February 1998.

P. Minovic, S. Ishikawa, K. Kato, “Symmetry Identification of a 3-D Object Represented by Octree”, IEEE Transactions on Pattern Analysis and Machine Intelligence, v. 15 n. 5, p. 507-514, May 1993.

Changming Sun, Jamie Sherrah, “3D Symmetry Detection Using The Extended Gaussian Image”, IEEE Transactions on Pattern Analysis and Machine Intelligence, v. 19 n. 2, p. 164-168, February 1997.

P. C. Marais, R. Guillemaud, M. Sakuma, A. Zisserman, and M. Brady, “Visualising cerebral asymmetry,” in Lecture Notes in Computer Science, K. H. Hohne and R. Kikinis, Eds, Hamburg, Germany: Springer, September 1996.

J. Udupa, D. Metaxas, Y. Jin, E. Angelini, T. Chen, Y Zhuge, “Hybrid Segmentation Methods”, book chapter in “Insight into Images: Principles and Practice for Segmentation, Registration, and Image Analysis”, edited by T. Yoo, A. K. Peters, March 2004.

Atam P. Dhawan, “Image registration”, book chapter in “Medical Image Analysis”, p 254-p 259, published by John Wiley & Sons, 2003.

Sylvain Prima, Sebastien Ourselin, and Nicholas Ayache, “Computation of the Mid-Sagittal Plane In 3-D Brain Images: IEEE transaction on medical imaging, vol. 21, no. 2, February 2002.

Celina Imielinska, Xin Liu, Joel Rosiene et al., “Towards Objective Quantification of Perfusion-Weighted Computed Tomography in the Setting of Subarachnoid Hemorrhage: Quantification of Symmetry and Automated Delineation of Vascular Territories”, Journal of Academic Radiology, 2005.

M. E. Brummer, “Hough transform detection of the longitudinal fissure in tomographic head images,” IEEE Trans. Med. Imag, vol. 10, pp. 74-81, March 1991.

B. A. Ardekani, J. Kershaw, M. Braun, and I. Kanno, “Automatic detection of the mid-sagittal plane in 3-D brain images,” IEEE Trans. Med Imag., vol. 16, pp. 947-952, December 1997.

C. Sun and J. Sherrah, “3D symmetry detection using the extended gaussian image,” IEEE Trans. Pattern Anal. Machine Intell., vol. 19, pp. 164-168, February 1997.

Y. Liu, R. T. Collins, and W. E. Rothfus, “Robust Midsagittal Plane Extraction from Normal and Pathological 3D Neuroradiology Images” IEEE Transactions on Medical Imaging, Vol. 20, No. 3, March, 2001, pp. 175-192.

P. Minovic, S. Ishikawa, K. Kato, “Symmetry Identification of a 3-D Object Represented by Octree”, IEEE Transactions on Pattern Analysis and Machine Intelligence, v. 15 n. 5, p. 507-514, May 1993.

P. C. Marais, R. Guillemaud, M. Sakuma, A. Zisserman, and M. Brady, “Visualising cerebral asymmetry,” in Lecture Notes in Computer Science K. H. Hohne and R. Kikinis, Eds, Hamburg, Germany: Springer, September 1996.

What is claimed:

1. A method for evaluating a medical image represented by image data, the method comprising:
assigning an axis of symmetry to a medical image;
computing, using the image data, at least one relative difference map based on a comparison of two substantially symmetrical areas around the axis of symmetry; and
generating a representation of any difference between the two symmetrical areas.

2. The method of claim 1, comprising scanning a region of interest to acquire the image data.

3. The method of claim 2, wherein scanning comprises performing a computed tomography scan.

4. The method of claim 1, wherein computing comprises generating at least one difference map.

5. The method of claim 1, wherein generating comprises generating a three dimensional color image illustrating the relative difference between the two substantially symmetrical areas.

6. The method of claim 1, wherein generating comprises generating a histogram representing the relative difference between the two substantially symmetrical areas.

7. The method of claim 1, wherein said assigning comprises a user assigning the axis of symmetry through a user interface.

8. The method of claim 1, wherein assigning comprises automatically assigning the axis of symmetry based on the image data.

9. The method of claim 1, wherein said computing comprises computing a statistical discrepancy between the two substantially symmetrical areas.

10. The method of claim 9, wherein said computing comprises using a Kolmogorov-Smimov test to compute the statistical discrepancy between the two substantially symmetrical areas.

11. The method of claim 9, further comprising defining at least two windows in the image data, each window representing one of the symmetrical areas for which at least one relative difference map is to be computed.

12. The method of claim 11, wherein said defining the windows comprises positioning each window in substantially equidistant locations from the assigned axis of symmetry.

13. The method of claim 11, wherein said defining the windows comprises defining the windows as having n×n pixels of the image data.

14. The method of claim 13, where n is equal to one of 9, 11, 13 and 15.

15. The method of claim 1, further comprising repeating said computing and generating for a second set of substantially symmetrical areas around the axis of symmetry to generate a second relative difference map.

16. A computer readable medium storing program code which, when executed, causes a computer to perform a method for evaluating a medical image represented by image data, the method comprising:
assigning an axis of symmetry to a medical image;
computing, using the image data, at least one relative difference map based on a comparison of two substantially symmetrical areas around the axis of symmetry; and
generating a representation of any difference between the two symmetrical areas.

17. A computer readable medium storing a data structure representing a relative difference map, the data structure comprising a quantification of statistical differences between image data values taken from corresponding value windows located substantially symmetrically with respect to an assigned axis of symmetry in a medical image.

18. A method for evaluating the symmetry of an image represented by image data, comprising: computing a shape of a substantially symmetrical object of interest based on image data, the object of interest having at least two substantially symmetrical sections;
assigning an axis of symmetry to the object of interest such that the axis lies between the two substantially symmetrical sections;
optionally converting the shape of the object of interest to a substantially rectangular or square shape;
optionally normalizing the converted shape;
determining, using the image and shape information, a degree of symmetry between the at least two substantially symmetrical sections with respect to the axis of symmetry; and
generating a graphical representation of any difference between the two substantially symmetrical sections.

19. The method of claim 18 wherein said computing further comprises using a bounding function to compute the shape of the substantially symmetrical object of interest.

20. The method of claim 18 wherein said determining further comprises performing a pixel comparison of the image and shape information to determine the degree of symmetry.

21. The method of claim 18 wherein said computing further comprises using a Fourier shape descriptor to compute the shape of the substantially symmetrical object of interest.

22. The method of claim 18 wherein said assigning further comprises computing at least one centroid to define the axis of symmetry.

23. A method of automatically identifying a plane of symmetry and correcting 3D orientation of volumetric images, comprising:
transforming a volume into a binary volume;
resampling the volume at a higher resolution;
computing a centroid and a covariance matrix of the volume;
forming an inertia matrix from the covariance matrix;
deriving principle axes of the volume form the inertia matrix; and
using the eigenvectors of the inertia matrix to obtain rotational angles of the mid-saggital plane of the volume.

24. The method of claim 23, wherein the volume is a volumetric image of a mammalian brain.

25. The method of claim 23, further comprising performing an affine spatial transformation for tilt correction after the principal axes have been derived.

25. The method of claim 25, wherein re-slicing is conducted on a re-centered and reoriented volume.

26. The method of claim 22, wherein after resampling a series of unique cubic polynomials is fitted between each of the data points.

27. A method, comprising:
representing a volumetric object as a re-parameterized surface point cloud;
removing the interior contents of the object; and
implementing a search for a best matching surface in a multi-resolution paradigm.

28. The method of claim 27, wherein the volume is a volumetric image of a mammalian brain.

29. The method of claim 27, wherein each location in the re-parameterized surface point cloud is parameterized by its elevation, azimuth and longitude.

30. The method of claim 27, further comprising performing an affine spatial transformation for tilt correction after the principal axes have been derived.

31. The method of claim 27, wherein re-slicing is conducted on a re-centered and reoriented volume.

32. A system for automatically identifying a plane of symmetry and correcting 3D orientation of volumetric images, comprising:
a binarizing module for transforming volume into a binary volume;
a resampling module for resampling the volume at a higher resolution;
a first computation module for computing a centroid and a covariance matrix of the volume;
an inertial matrix module for forming an inertia matrix from the covariance matrix and deriving principle axes of the volume form the inertia matrix; and
a second computational module for using the eigenvectors of the inertia matrix to obtain rotational angles of the mid-saggital plane of the volume.

33. A volumetric image analysis system, comprising:
a re-parameterization module for representing a volumetric object as a re-parameterized surface point cloud;
a de-interiorization module for removing the interior contents of the object; and
a surface matching module for implementing a search for a best matching surface in a multi-resolution paradigm.

34. The method of claim 33, wherein after said fitting of polynomials the volume is downsampled.
